(19) United States
(12) Patent Application Publication
(10) Pub. No.: US 2026/0209957 A1
(43) Pub. Date: Jul. 23, 2026

(54) Sequential one-pot synthesis for preparing 4-(4-cyano-2-methoxyphenyl)-5-ethoxy-2,8-dimethyl-1,4-dihydro-1,6-naphthyridine-3-carboxamide

(71) Applicant: BAYER AKTIENGESELLSCHAFT, Leverkusen (DE)

(72) Inventors: Jens AßMANN, Haan (DE); Johannes PLATZEK, Berlin (DE); Marc NOWAKOWSKI, Wuppertal (DE); Dirk STORCH, Köln (DE)

(21) Appl. No.: 19/138,918

(22) PCT Filed: Dec. 14, 2023
(86) PCT No.: PCT/EP2023/085843

§ 371 Date: Jun. 13, 2025

(30) Foreign Application Priority Data
Dec. 16, 2022 (EP) 22214224.2
Jul. 19, 2023 (EP) 23186372.1

(51) Int. Cl. C25B 3/05; C07D 471/04; C25B 3/07; C25B 3/09; C25B 3/23; C25B 3/25

(52) U.S. Cl. CPC C25B 3/05; C07D 471/04; C25B 3/07; C25B 3/09; C25B 3/23; C25B 3/25

(57) ABSTRACT

The present invention covers a sequential one-pot synthesis for preparing 4-(4-cyano-2-methoxy-phenyl)-5-ethoxy-2,8-dimethyl-1,4-dihydro-1,6-naphthyridine-3-carboxamide according to formula (XIII). In particular, in the sequential one-pot synthesis (4R)-4-(4-cyano-2-methoxy-phenyl)-5-ethoxy-2,8-dimethyl-1,4-dihydro-1,6-naphthyridine-3-carboxamide according to formula ent-(I) is used as starting material to obtain 4-(4-cyano-2-methoxy-phenyl)-5-ethoxy-2,8-dimethyl-1,4-dihydro-1,6-naphthyridine-3-carboxamide according to formula (XIII) via electrochemical oxidation and electrochemical reduction.

The present invention covers a sequential one-pot synthesis for preparing 4-(4-cyano-2-methoxy-phenyl)-5-ethoxy-2,8-dimethyl-1,4-dihydro-1,6-naphthyridine-3-carboxamide according to formula (XIII)

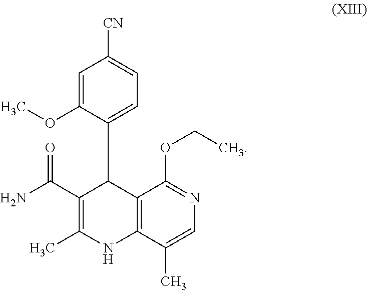 [SMILES: CCOc1ncc(C)c2c1C(c1ccc(C#N)cc1OC)C(C(N)=O)=C(C)N2]

In particular, in the sequential one-pot synthesis the compound according to formula ent-(I)

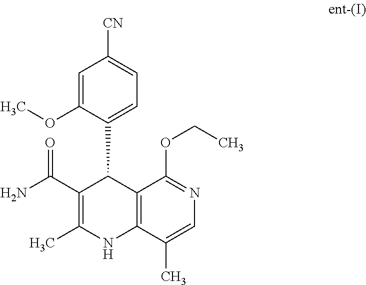 [SMILES: CCOc1ncc(C)c2c1[C@@H](c1ccc(C#N)cc1OC)C(C(N)=O)=C(C)N2]

is used as starting material to obtain compound (XIII) via electrochemical oxidation and electrochemical reduction. Compound (XIII) is an intermediate in the synthesis of compound (4S)-4-(4-cyano-2-methoxyphenyl)-5-ethoxy-2,8-dimethyl-1,4-dihydro-1,6-naphthyridine-3-carboxamide of formula (I)

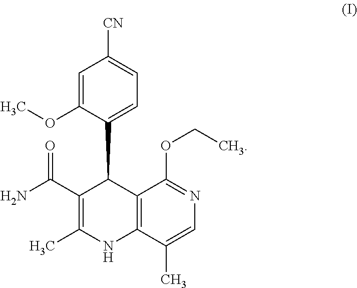 [SMILES: CCOc1ncc(C)c2c1[C@H](c1ccc(C#N)cc1OC)C(C(N)=O)=C(C)N2]

The International Nonproprietary Name (INN) of the compound of the formula (I) is finerenone. It is a non-steroidal antagonist of the mineralocorticoid receptor and may be used as an agent for prophylaxis or treatment of cardiovascular or renal disorders such as heart failure or diabetic nephropathy. The compound (I) and the preparation process thereof are described in for example US20100136142 A1, US20170217957 A1 and U.S. Ser. No. 15/753,406 A1.

The compound ent-(I) is a by-product or waste product that occurs in the preparation of finerenone (I). In the commercial synthesis of finerenone, the compound ent-(I) is not further used and has to be destroyed/disposed. The disposal of such waste is a complex and cost-intensive process and means a high inefficiency in respect of the yield of finerenone. In addition, the compound is obtained in significant quantities at one of the last stages of the commercial synthesis, so that potential starting material is discarded here.

U.S. Ser. No. 15/753,406 A1 describes that in the preparation of compound (I), the compound according to formula ent-(I) is obtained (see scheme 1).

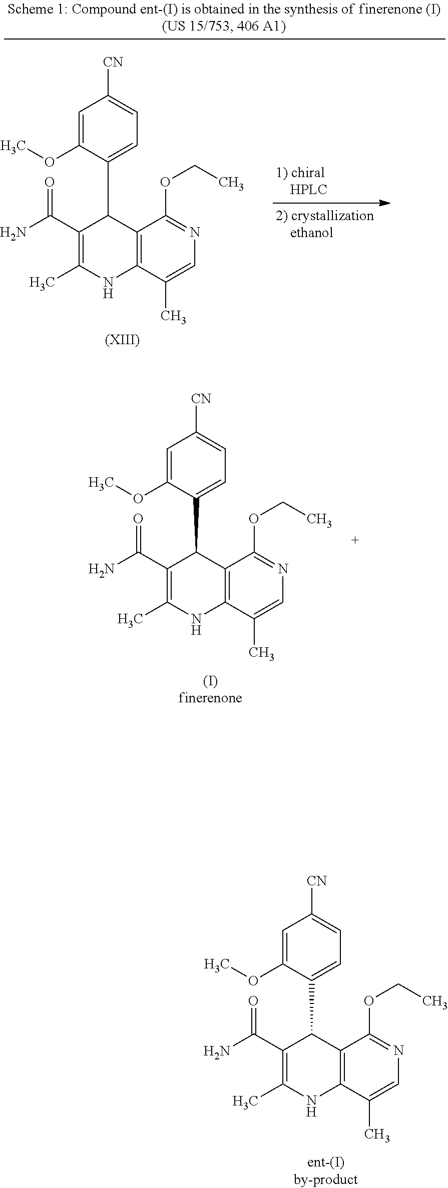

U.S. Ser. No. 15/753,406 A1 further describes a recycling process for compound ent-(I) in lab scale. The process of U.S. Ser. No. 15/753,406 A1 is depicted in scheme 2 below and comprises several separate steps.

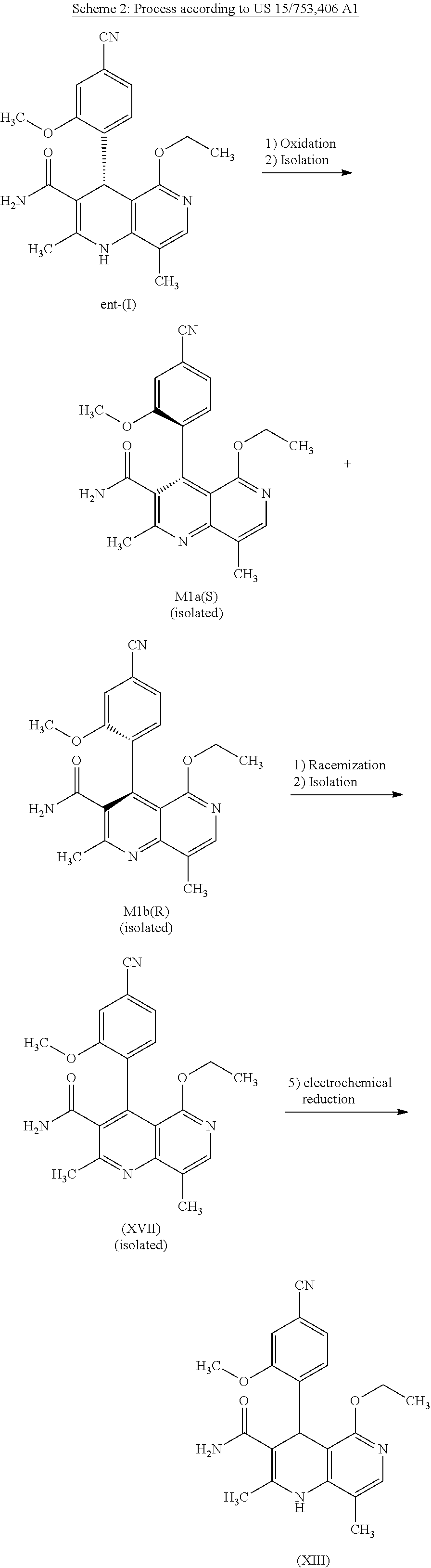

The process described in U.S. Ser. No. 15/753,406 A1 is characterized by the following steps, each of which is characterized by isolation of the corresponding intermediate product as a solid:
1) oxidation with chemical oxidizing agents to the compounds M1a(S) and M1b(R)
2) isolation of the mixture of compounds M1a(S) and M1b(R)
3) racemization of the compounds M1a(S) and M1b(R) to compound (XVII)
4) isolation of compound (XVII)
5) electrochemical reduction of compound (XVII) to give compound (XIII).

In Table 1 an overview of the yields after each step and the overall yield after all reaction steps (lab scale) described in U.S. Ser. No. 15/753,406 A1 are given.

TABLE 1
Yield (%) after oxidation, yield (%) after racemization, yield (%) after
reduction and overall yield of the synthesis described in US 15/753,406
A1 (US′406)

| | | Yield (%) | | |
|---|---|---|---|---|
| | Yield (%) | after | Yield (%) | |
| | after oxidation | racemization | after reduction | |
| | and isolation | and isolation | and isolation | Overall yield |
| US′406 A1 | 86.7 % | 85.9 % | 85 % | 64 % |
| | of theory | of theory | of theory | of theory |
| Example in | Example 12b | Example 12b | Example 32 | isolated yield |
| US′406 | (HNO₃ | | (100 cm² cell) | |
| | oxidation) | | | |

The process described in U.S. Ser. No. 15/753,406 A1 has some disadvantages. For example:
It only refers to lab scales and process settings that are not suitable for a commercial scale-up
That the procedure described in U.S. Ser. No. 15/753,406 A1 is not suitable can also be seen e.g. in Example 28 of U.S. Ser. No. 15/753,406 A1: Here, the direct electrochemical oxidation failed.
The respective intermediates have to be isolated after each individual step.
The direct electrochemical oxidation does not need those reagents.
Undesirable secondary components can arise.
Undesirable secondary components can significantly interfere with the further process steps.
Undesirable components are oxidizing agents and solvents can significantly interfere with the further process steps.
Any arising secondary component (e.g., an impurity due to low chemical selectivity) should be purged in the crystallization/isolation step.
A secondary component could be the reduced form of the chemical oxidizing agent (oxidation with Fe(3+) leads to Fe(2+), or DDQ takes up to H-Atoms and forms H2DDQ), however, the component itself (independent of its oxidation state) might be an issue for reaching high selectivity/yield in the next steps.
The required isolation step(s) of the intermediate(s) result in at least two specific disadvantages:
   additional process steps such as filtration, washing, drying, and repeated solids handling (e.g., filling of dried solids into containers, intermediate storage and supply of stored materials towards next synthesis step)
   yield losses when isolating the intermediate, for example, via the mother liquors or washing steps
The dihydropyridine derivatives in U.S. Ser. No. 15/753,406 A1 are oxidized to their pyridine analogues using indirect electrochemical oxidation with sub-stoichiometric amounts of mediators (e.g. DDQ, see also U.S. Ser. No. 15/753,406 A1, FIG. 4). Such mediators are also described in Francke and Little, Chem. Soc. Rev. 43(8), 2014, pages 2492-2521. These oxidizing agent(s) used in oxidation step cat least can interfere with further process steps or may even be limiting the electrochemical reduction step.
The solvents used in the oxidation step of U.S. Ser. No. 15/753,406 A1
The synthesis is unsuitable for a large-scale process, since many steps proceed at very high dilution, with very high excesses of reagents and therefore afford a relatively low overall yield. Furthermore, many intermediate chromatographic purifications and/or isolation steps are necessary, which are technically generally very laborious and entail a high consumption of solvents, are costly and are therefore to be avoided if possible. Some stages are not achievable due to safety and process technology difficulties.

This list is not exhaustive.

Thus, there exists a need for an industrially practicable and/or large-scale synthesis, which delivers the compound according to formula (XIII) in a reproducible manner, minimum of reaction steps, minimum of isolation steps, in high overall yield, low production costs and high purity. High demands are placed on the purity of medicinal substances and their intermediates. For example, these need to meet all regulatory requirements, in order to be suitable for clinical trials, later regulatory submission and/or for the final application to a patient. Example of such regulations are Good Manufacturing Practice (GMP) and Good Clinical Practice (GCP).

Surprisingly, a sequential one-pot synthesis has been found which allows the requirements mentioned above to be met.

The invention covers a sequential one-pot synthesis for synthesizing 4-(4-cyano-2-methoxy-phenyl)-5-ethoxy-2,8-dimethyl-1,4-dihydro-1,6-naphthyridine-3-carboxamide according to formula (XIII)

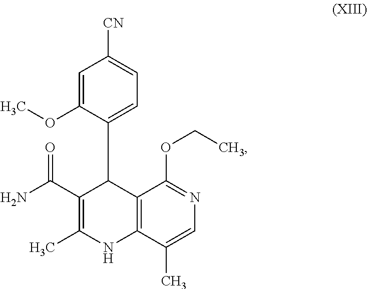 [SMILES: CCOc1ncc(C)c2c1C(c1ccc(C#N)cc1OC)C(C(N)=O)=C(C)N2]

the synthesis comprising the steps of:
step a) synthesizing 4-(4-cyano-2-methoxy-phenyl)-5-ethoxy-2,8-dimethyl-1,6-naphthyridine-3-carboxamide according to formula (XVII)

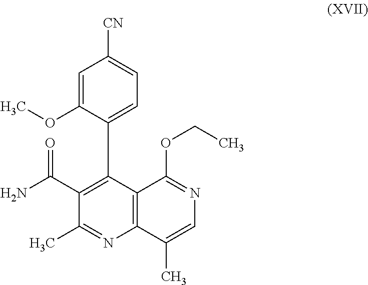 [SMILES: CCOc1ncc(C)c2nc(C)c(C(N)=O)c(-c3ccc(C#N)cc3OC)c12]

via electrochemical oxidation of (4R)-4-(4-cyano-2-methoxy-phenyl)-5-ethoxy-2,8-dimethyl-1,4-dihydro-1,6-naphthyridine-3-carboxamide according to formula ent-(I)

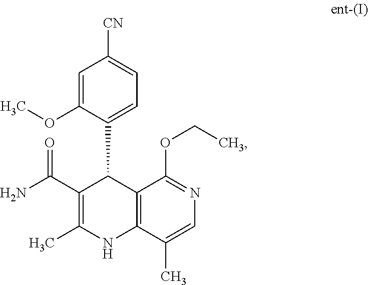 [SMILES: CCOc1ncc(C)c2c1[C@@H](c1ccc(C#N)cc1OC)C(C(N)=O)=C(C)N2]

step b) synthesizing 4-(4-cyano-2-methoxy-phenyl)-5-ethoxy-2,8-dimethyl-1,4-dihydro-1,6-naphthyridine-3-carboxamide according to formula (XIII)

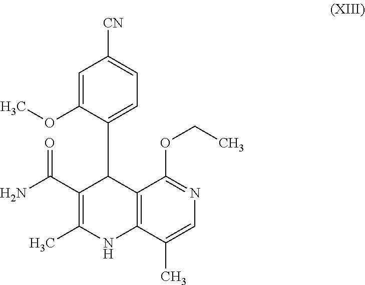 [SMILES: CCOc1ncc(C)c2c1C(c1ccc(C#N)cc1OC)C(C(N)=O)=C(C)N2]

via electrochemical reduction of the compound according to formula (XVII).

Moreover, with the sequential one-pot synthesis, it has been made possible to produce the compound of the formula (XIII) in a very efficient manner. The sequential one-pot synthesis offers significant advantages over the prior art in terms of scalability and technical implementation. The overall yield is significantly higher compared to the previously described synthesis.

In particular, the invention covers a sequential one-pot synthesis as depicted in scheme 3 below:

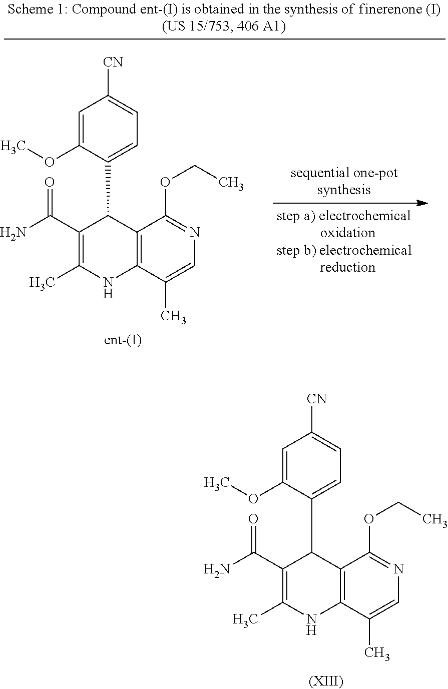

In contrast to the known process, the synthesis according to the invention is conducted as one-pot synthesis, in which first the electrochemical oxidation (step a)) and then the electrochemical reduction (step b)) is conducted. In the sequential one-pot synthesis there is no need for isolation of the intermediates. The sequential one-pot synthesis can be run in one set-up/apparatus. For example, the sequential one-pot synthesis can be run in one of the set-ups/apparatuses as depicted in FIG. 1 or FIG. 2 below. The electrochemical oxidation and electrochemical reduction can thus be carried out directly one after the other in one apparatus/set-up. The sequential one-pot synthesis can be run without isolation of the intermediates as required in the known processes. In contrast thereto, in the known process the intermediates (compounds M1a(S), M1b(R), and/or (XVII)) have to be isolated as explained above.

Even if one-pot syntheses are known, the already known synthesis of U.S. Ser. No. 15/753,406 A1 could not have been scaled-up and/or carried out as a one-pot synthesis without further ado. The reaction conditions of the single steps described in U.S. Ser. No. 15/753,406 A1 differ significant from each other. The different steps each require different reaction conditions. The reaction condition of the previous step may be unsuitable for the subsequent step. For example, the oxidation step in U.S. Ser. No. 15/753,406 A1 is carried out using nitric acid and acetonitrile. These reaction conditions are not suitable for the subsequent thermal racemization on industrial scale for at least safety reasons. In U.S. Ser. No. 15/753,406 A1, the oxidizing reagents were used in excess. In presence of remaining oxidizing reagents, consecutive oxidation reactions at elevated temperatures may occur and cause formation of new impurities. This can lead to a lower quality and yield.

In addition, the sequential one-pot synthesis allows a re-cycling of the compound according to formula (XIII) from compound ent-(I) in industrial scale. Compound (XIII) can then be subjected again to an enantiomeric resolution to obtain compound (I). See e.g. scheme 4.

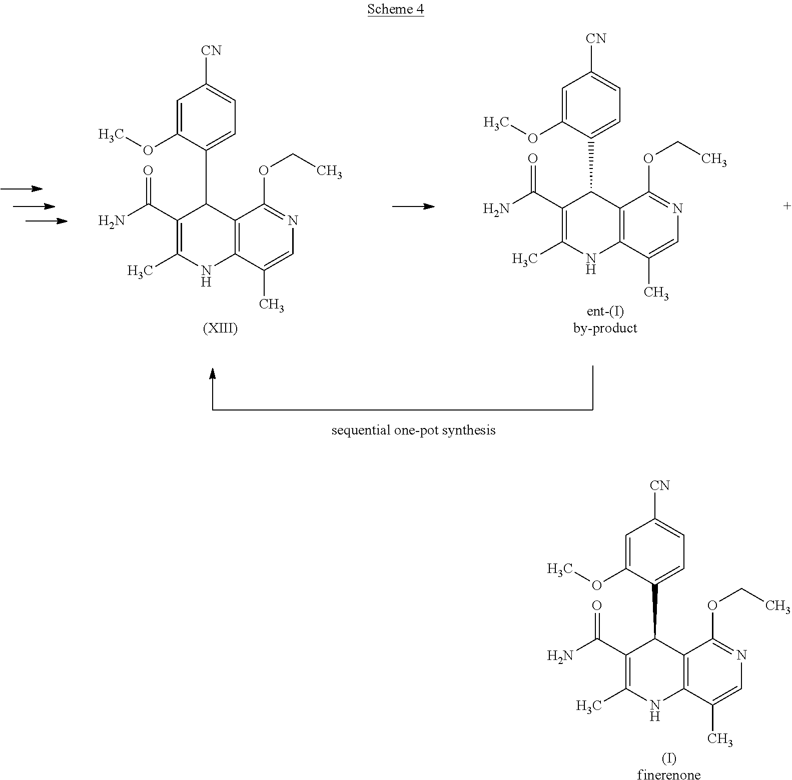

Thus, the by-product according to formula ent-(I) does not need to be destroyed but can be re-used. This is very sustainable, because there is less chemical waste that needs to be disposed of separately and/or valuable starting material/intermediates can be recovered at one of the final stages of the process. Since this is a large-scale process, this is not only good for the environment, but also saves money, time and material. This is very advantageous for reasons of cost and, on the other hand, for reasons of sustainability, especially since it is a large-scale process.

The sequential one-pot synthesis described here can be carried out several times in succession and, thus, offers the possibility of converting the compound ent-(I) into compound (XIII). This can be seen as a quasi-continuous mode of operation, which offers great advantages in terms of costs, time and/or resources. In this way, the waste product ent-(I) that occurs again and again in the preparation of finerenone (I) can be converted back into the compound (XIII). The compound (XIII) can then in turn be fed back into the production process of finerenone (I). After several process cycles of the sequential one-pot synthesis, the compound ent-(I) can, thus, be almost completely utilized. In the best case, the by-product ent-(I) can be almost completely recycled to the envisaged product (XIII) or finerenone (I).

The sequential one-pot synthesis enables to produce the compound of the formula (XIII) in a reproducible, sustainable and/or economical manner. After several process cycles of the sequential one-pot synthesis, the compound ent-(I) can almost be completely utilized.

The compound (XIII) can then e.g. be used for the preparation of finerenone by e.g. chiral chromatography, classical resolution by diastereomeric salt formation, crystallization, precipitation and so on. Enantiomeric HPLC is known from e.g. US20100136142 A1 and US20170217957 A1. Separation of the enantiomers via diastereomeric separation is e.g. described in US20210163474 A1.

A further particularly important advantage of the invention is that the compound of the formula (XIII) can be recovered in a high chemical purity. Since it is an active pharmaceutical ingredient/intermediate, all operations are carried out under GMP and require high purity of the intermediates.

It was also surprising that the compound of the formula (XIII) can be recovered via electrolysis in a large scale. Commercially, electrolytic cells are used in the electrorefining and electrowinning of several non-ferrous metals. Almost all high-purity aluminum, copper, zinc, and lead are produced industrially in electrolytic cells. However, in pharmaceutical industry the use of such electrolysis cells for synthesizing drugs is not common. This is even true for large scale processes. Industrial production of active ingredients takes place in so-called “multipurpose plants”. There is only standard equipment (comprising e.g. boilers, centrifuges, dryers, . . . ). The use of electrolysis cells in such multipurpose plants in pharmaceutical industry is neither a standard set-up, nor typically used for commercial synthesis in the pharma industry.

Unless defined otherwise, the technical terms used herein are used in the manner customary for the person skilled in the art. The nomenclature follows International Union of Pure and Applied Chemistry (IUPAC).

The units used herein are in accordance with International System of Units (SI units).

4-(4-cyano-2-methoxy-phenyl)-5-ethoxy-2,8-dimethyl-1,4-dihydro-1,6-naphthyridine-3-carboxamide is the compound according to formula (XIII)

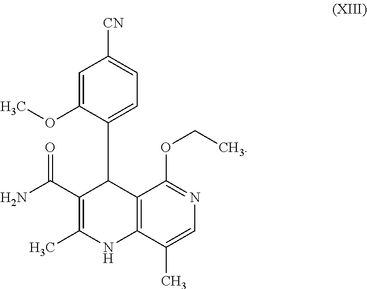 [SMILES: CCOc1ncc(C)c2c1C(c1ccc(C#N)cc1OC)C(C(N)=O)=C(C)N2]

The terms “4-(4-cyano-2-methoxy-phenyl)-5-ethoxy-2,8-dimethyl-1,4-dihydro-1,6-naphthyridine-3-carboxamide”, “The compound according to formula (XIII)” and “compound (XIII)” are synonyms. The compound according to formula (XIII) comprises (4S)-4-(4-cyano-2-methoxyphenyl)-5-ethoxy-2,8-dimethyl-1,4-dihydro-1,6-naphthyridine-3-carboxamide of formula (I) and (4R)-4-(4-cyano-2-methoxy-phenyl)-5-ethoxy-2,8-dimethyl-1,4-dihydro-1,6-naphthyridine-3-carboxamide according to formula ent-(I)

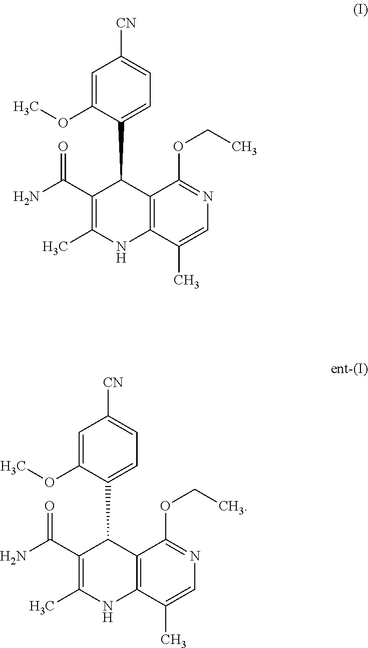

(4S)-4-(4-cyano-2-methoxyphenyl)-5-ethoxy-2,8-dimethyl-1,4-dihydro-1,6-naphthyridine-3-carboxamide is the compound according to formula (I)

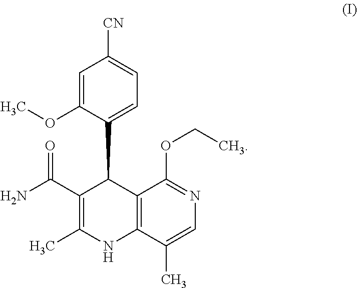 [SMILES: CCOc1ncc(C)c2c1[C@H](c1ccc(C#N)cc1OC)C(C(N)=O)=C(C)N2]

The INN of compound (I) is “Finerenone”. The CAS number of Finerenone is CAS 1050477-31-0. Compound (I) is the (S)-enantiomer comprised in the compound according to formula (XIII). The terms (4S)-4-(4-cyano-2-methoxyphenyl)-5-ethoxy-2,8-dimethyl-1,4-dihydro-1,6-naphthyridine-3-carboxamide”, “finerenone”, and “compound (I)” are synonyms.

(4R)-4-(4-cyano-2-methoxy-phenyl)-5-ethoxy-2,8-dimethyl-1,4-dihydro-1,6-naphthyridine-3-carboxamide is the compound according to formula ent-(I)

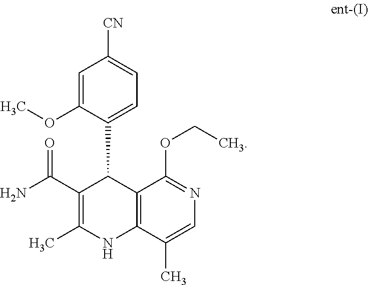 [SMILES: CCOc1ncc(C)c2c1[C@@H](c1ccc(C#N)cc1OC)C(C(N)=O)=C(C)N2]

Compound ent-(I) is described in U.S. Ser. No. 15/753,406 A1. Compound ent-(I) is the (R)-enantiomer comprised in the compound according to formula (XIII). “(4R)-4-(4-cyano-2-methoxy-phenyl)-5-ethoxy-2,8-dimethyl-1,4-dihydro-1,6-naphthyridine-3-carboxamide”, “compound according to formula ent-(I)” and “compound ent-(I)” are synonyms. The compound ent-(I) can be obtained as waste product or by-product in the synthesis of finerenone (I) as depicted in scheme 1 above.

### Compounds M1a(S) and M1b(R)

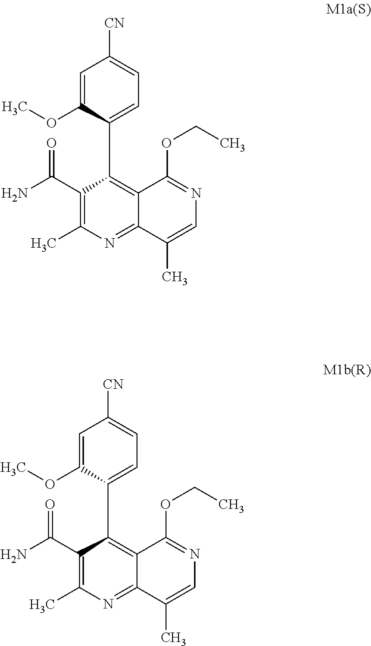

are described in U.S. Ser. No. 15/753,406 A1. These compounds are atropisomers comprised in compound (XVII). 4-(4-cyano-2-methoxy-phenyl)-5-ethoxy-2,8-dimethyl-1,6-naphthyridine-3-carboxamide according to formula (XVII)

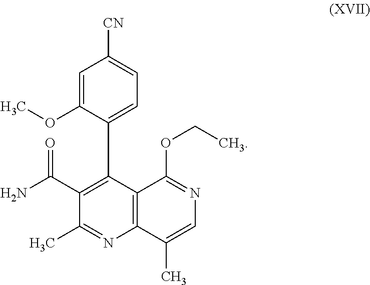 [SMILES: CCOc1ncc(C)c2nc(C)c(C(N)=O)c(-c3ccc(C#N)cc3OC)c12]

Thus, compound (XVII) comprises compounds M1a(S) and M1b(R).

The term “intermediates” can refer to the intermediates of the sequential one-pot synthesis for preparing the compound according to formula (XIII) or the process for the preparation the compound according to formula (I). Intermediates can be e.g. the compound according to formula ent-(I), the compound according to formula M1a(S), the compound according to formula M1b(R), the compound according to formula (XVII), the compound according to formula (XIII).

The term “product” can refer to the compound according to formula (XIII) or the compound according to formula (I). It may depend to which aspect or partial aspect of the processes or steps described herein it is referred to. A “conducting salt” or “conducting electrolyte” is a salt that takes over charge transport during electrolysis. The conducting salt reduces the Ohmic resistance of the solution and causes the depolarizers to be transported to the electrodes by diffusion. It does not participate in the electrode reactions. An Examples of conducting salts are tetrabutylammonium perchlorate BuN₄ClO₄, Et₄NBF₄, Bu₄NBF₄, Bu₄NPF₆, Bu₄NX (with X═I, Br) or perchlorates NaClO₄, LiClO₄, Et₄NClO₄. These conducting salts can be used in the sequential one-pot synthesis according to the invention.

The term “solvent” also includes solvent mixtures. Solvents can be recycled and also reused as mixtures. In one embodiment, the solvent is recycled. In another embodiment, the solvent is recycled and the component produced in the surplus can then be supplemented with fresh solvent in order to obtain the desired mixing ratio.

In one embodiment, a suspension or a mixture thereof is used as a solvent. The use of a suspension or mixture thereof can lead to an optimal space-time yield. Suspensions can be transported through pumps through the system/process set-up used.

In one embodiment of the invention, the sequential one-pot synthesis is conducted via electrolysis. The reaction type “electrolysis” is also known in the art. The chemical changes caused by the passage of current through an electrolyte are called electrolysis. Electrical energy is converted directly into chemical energy. Electrolytic processes are electrochemical reactions in which material conversion processes that do not take place voluntarily are forced by applying an external voltage. Electrical energy (current) is converted into chemical energy (substances). Electrolysis is the reversal of galvanic processes by performing electrical work.

Electrolysis apparatuses or cells are known to those skilled [Encyclopedia of Applied Electrochemistry, G. Kreysa et al. (eds.), Encyclopedia of Applied Electrochemistry, DOI 10.1007/978-1-4419-6996-5, Springer Science and Business Media New York 2014, pp. 568-578; Electrochmica Acta 161, (2015), pp. 436-451; Journal of Applied Electrochemistry 27 (1997), p. 1313, p. 1322]. Electrolysis apparatuses are also described in U.S. Ser. No. 15/753,406 A1. Electrochemical cells for industrial scale can comprise one or more electrochemical cells. Such industrial scale arrangements can comprise one are more electrochemical cells, compartments, vessels and/or tanks and the like. Such industrial scale systems often consist of or comprise several electrochemical cells. These can be connected in or run parallel. Such industrial systems can include one or more tanks, reaction vessels or the like. The reaction mixture can be telescoped between the respective compartments of such industrial scale system. Accordingly, in such industrial scale systems the reaction steps according to the invention can be carried out sequentially.

In one embodiment of the invention, the sequential one-pot synthesis is an electrochemical synthesis. In one embodiment of the invention, the sequential one-pot synthesis is carried out in an electrochemical cell. In one embodiment of the invention, the sequential one-pot synthesis is carried out in an electrolytic cell. Electrolytic cells are known in the art. An electrolytic cell is an electrochemical cell that utilizes an external source of electrical energy to drive a chemical reaction that would not otherwise occur. Voltage is applied between two electrodes i.e. anode (positively charged electrode) and cathode (negatively charged electrodes). This is in contrast to a galvanic cell, which itself is a source of electrical energy. In an electrolytic cell, a current passes through the cell by an external voltage, causing a non-spontaneous chemical reaction to proceed. An electrolytic cell has three components: an electrolyte and two electrodes (a cathode and an anode). The electrolyte is usually a solution of water or other solvents in which ions are dissolved. When driven by an external voltage applied to the electrodes, the ions in the electrolyte are attracted to an electrode with the opposite charge, where charge-transferring (also called faradaic or redox) reactions can take place. Only with an external electrical potential (i.e., voltage) of correct polarity and sufficient magnitude can an electrolytic cell decompose a normally stable, or inert chemical compound in the solution. The electrical energy provided can produce a chemical reaction that would not otherwise occur spontaneously (non-spontaneous reaction).

FIG. 1 and FIG. 2 schematically show a set-up of an electrolytic apparatus. Such electrolytic apparatuses can be used for large-scale synthesis. The electrolysis apparatuses shown in FIG. 1 and FIG. 2 can also be used for the sequential one-pot synthesis according to the invention. Further modifications to such electrolytic apparatuses can be made. For example, several electrolyte tanks and/or anolyte tanks can be used. Other devices such as separators (e.g. for gas), heat exchangers, heating devices, cooling devices and so on can also be used/integrated. Corresponding set-up which are suitable for an industrial-scale synthesis are known to the person skilled in the art.

In one embodiment, the sequential one-pot synthesis is carried out at in a divided cell. In one embodiment, the sequential one-pot synthesis is carried out in a filter-press type cell. Other commercially available cells can be used and are known to the skilled person.

Further modifications to such electrolytic apparatuses can be made. The modifications or adaptions described below can be applied to the schematic standard set-up depicted in FIGS. 1 and/or 2. For example, several electrolyte tanks and/or anolyte tanks can be used. Other devices such as separators (e.g. for gas), heat exchangers, heating devices, cooling devices and so on can also be used/integrated. Corresponding set-up which are suitable for an industrial-scale synthesis are known to the person skilled in the art. The process can be entirely and/or partially worked in a continuous manner and/or discontinuous manner. Filter units for the for solids can be installed. With such filters e.g. starting material, intermediates (e.g. ent-(I), M1a(S), M1b(R), (XVII), (XIII)) or products (e.g. (XIII), (I)) can isolated or separated from the reaction mixture.

These filter units can be cooled, the filtrate would then be returned to the process. Furthermore, dwell loops that could be thermally heated or cooled. This could lead to a continuous racemization. It is also possible to install continuous dosing units. Suspension pumps can also be installed. Electrolysis cells can contain, comprise and/or be made (partially) of glass, enamel or stainless steel optionally with Teflon inliner or plastic with non ‘leachable’ properties (GMP approved).

The electrode geometry can also be adapted to achieve an optimal flow. The electrode geometry can also be adapted to achieve an optimized flow so that gas formation and discharge can be controlled. Heat dissipation measures can be performed to match to the respective electrode geometry. The fluid distribution in the electrolysis cell or apparatus or process setting can be further adapted. Such adaptations can be realized e.g. through additional stirring measures for better mass transport.

Different types of electrodes can be used. Examples are electrodes comprising or containing or consisting of precious metal, coated with precious metals, titanium electrodes, graphite electrodes, boron-doped diamond electrodes (BDD). The electrodes can comprise or consist of precious metals, metal or non-metal supports coated with precious metals and/or non-metal electrodes (e.g. graphite).

Commercial membranes can be used to realize a split cell.

The electrodes can be segmented. The electrodes can be segmented, and the total electrode area of the can be 60 to 100 cm². In one embodiment the electrodes are segmented. In one embodiment the total electrode area of is 60 to 100 cm². In one embodiment the electrodes are segmented and the total electrode area of is 60 to 100 cm². In one embodiment the electrode area is 80 cm (wide) and 25 to 75 cm (high).

The electrolysis cell can consist or comprise a segmented cell unit, which can be expandable by a “numbering-up” in the stack design.

The optimization of existing material and sealing concepts, the improvement of electrode structure and cell geometries as well as the adaptation of system components, e.g. for heat dissipation and control, can be optimally adapted by adjusting the voltage, current densities, temperatures and flow velocities/rates, and/or dwell times.

The process can be conducted under inert gas. Various inert gases are known to the skilled person. Examples are CO₂, N₂, Argon (Ar), Neon (Ne), Radon (Ra), Helium (He), Krypton (Kr), xenon (Xe), radon (Rn) and/or mixtures thereof. The inert gas used can be pure gas or a mixture thereof. A mixture of an inert gas and a non-inert gas can be used. In one embodiment this mixture comprises predominantly the inert gas.

The process described herein can be performed entirely and/or partially in different modes. Examples of such modes are campaign mode, or in continuous process mode.

In one embodiment, the process according to the invention is worked in a continuous mode. In one embodiment, the process according to the invention is worked in a continuous mode, wherein the starting material, the intermediates and/or the conducting salt is added continuously. In one embodiment, the process according to the invention is worked in a continuous mode, wherein
the starting material, solvent, conducting salt, acid, and/or intermediate is added continuously and
the product is removed continuously.

The cycle times for each step described below can be adjusted, respectively. One or more of these steps can be performed multiple times, with one or more of the steps being performed less often or even only once.

The improvement of the long-term stability can also be optimized. The long-term stability can be improved in which the dwell times are optimally adjusted. In one embodiment the dwell time is equal or less than 20 hours. In one embodiment the dwell time is 5 to 8 hours. In one embodiment the dwell time in the individual unit operation is equal or less than 6 hours. The dwell times should be adapted to the geometry and/or flow rates.

Multiphase flow generated by gas bubble formation can be suppressed. For this purpose, heat dissipation strategies and/or the influence of different geometries and/or process parameters on the operating behavior can be optimally designed.

### Step a)

The sequential one-pot synthesis comprises step a).

In one embodiment, step a) is conducted at temperatures selected from 15 to 150° C., 15 to 120° C., and 20 to 100° C. In one embodiment, step a) is conducted at temperatures from 15 to 150° C. In one embodiment, step a) is conducted at temperatures from 15 to 120° C. In one embodiment, step a) is conducted at temperatures from 20 to 100° C. In one embodiment, step a) is conducted at a temperature from 15 to 150° C. In one embodiment, step a) is conducted at a temperature from 15 to 120° C. In one embodiment, step a) is conducted at a temperature from 20 to 100° C. In one embodiment, step a) is conducted at ambient temperature.

In step a), the temperature can be constant, variable, increasing or decreasing. In step a), a specific temperature program can also be run. In one embodiment, step a) is conducted at a temperature from 15 to 150° C., and wherein the temperature is constant, variable, increasing, decreasing or a specific temperature program is run.

In step a), the temperature can be constant, variable, increasing or decreasing. In step a), a specific temperature program can also be run.

In one embodiment, step a) is conducted at temperatures from 15 to 150° C., and wherein the temperature is constant, variable, increasing, decreasing or a specific temperature program is run.

In one embodiment, step a) is conducted at a temperature from 15 to 150° C., wherein the temperature is constant. In one embodiment, step a) is conducted at temperatures from 15 to 120° C., wherein the temperature is constant. In one embodiment, step a) is conducted at temperatures from 20 to 100° C., wherein the temperature is constant.

In one embodiment, step a) is conducted at a temperature from 15 to 150° C., wherein the temperature is variable. In one embodiment, step a) is conducted at temperatures from 15 to 120° C., wherein the temperature is variable. In one embodiment, step a) is conducted at temperatures from 20 to 100° C., wherein the temperature is variable.

In one embodiment, step a) is conducted at a temperature from 15 to 150° C., wherein the temperature is increased. In one embodiment, step a) is conducted at temperatures from 15 to 120° C., wherein the temperature is increased. In one embodiment, step a) is conducted at temperatures from 20 to 100° C., wherein the temperature is increased.

In one embodiment, step a) is conducted at a temperature from 15 to 150° C., wherein the temperature is decreased. In one embodiment, step a) is conducted at temperatures from 15 to 120° C., wherein the temperature is decreased. In one embodiment, step a) is conducted at temperatures from 20 to 100° C., wherein the temperature is decreased.

In one embodiment, step a) is conducted a temperature from 15 to 150° C., wherein a specific temperature program is run. In one embodiment, step a) is conducted at temperatures from 15 to 120° C., wherein a specific temperature program is run. In one embodiment, step a) is conducted at temperatures from 20 to 100° C., wherein a specific temperature program is run.

In one embodiment of step a) first the electrochemical oxidation is conducted and then the reaction mixture is heated.

In one embodiment of step a) first the electrochemical oxidation is conducted and then the reaction mixture is heated to 50 to 150° C. In one embodiment of step a) first the electrochemical oxidation is conducted and then the reaction mixture is heated to 75 to 150° C. In one embodiment of step a) first the electrochemical oxidation is conducted and then the reaction mixture is heated to 80 to 150° C. In one embodiment of step a) first the electrochemical oxidation is conducted and then the reaction mixture is heated to 80 to 140° C. In one embodiment of step a) first the electrochemical oxidation is conducted and then the reaction mixture is heated to 90 to 120° C.

In one embodiment of step a) first the electrochemical oxidation is conducted, wherein the temperature can be constant, variable, increasing, decreasing or a specific temperature program can also be run, and then the reaction mixture is heated.

In one embodiment of step a), first the electrochemical oxidation is conducted, wherein the temperature can be constant, variable, increasing, decreasing or a specific temperature program can also be run, and wherein the reaction mixture is then heated to 50 to 150° C.

In one embodiment of step a), first the electrochemical oxidation is conducted, wherein the temperature can be constant, variable, increasing, decreasing or a specific temperature program can also be run, and wherein the reaction mixture is then heated to 75 to 150° C.

In one embodiment of step a), first the electrochemical oxidation is conducted, wherein the temperature can be constant, variable, increasing, decreasing or a specific temperature program can also be run, and wherein the reaction mixture is then heated to 80 to 150° C.

In one embodiment of step a), first the electrochemical oxidation is conducted, wherein the temperature can be constant, variable, increasing, decreasing or a specific temperature program can also be run, and wherein the reaction mixture is then heated to 80 to 140° C.

In one embodiment of step a), first the electrochemical oxidation is conducted, wherein the temperature can be constant, variable, increasing, decreasing or a specific temperature program can also be run, and wherein the reaction mixture is then heated to 90 to 120° C.

In one embodiment of step a) first the electrochemical oxidation is conducted at temperatures selected from 15 to 150° C., 15 to 120° C., and 20 to 100° C. and then the reaction mixture is heated.

In one embodiment of step a) first the electrochemical oxidation is conducted at temperatures selected from 15 to 150° C., 15 to 120° C., and 20 to 100° C., wherein the temperature can be constant, variable, increasing, decreasing or a specific temperature program can also be run, and then the reaction mixture is heated.

In one embodiment of step a) first the electrochemical oxidation is conducted at temperatures selected from 15 to 50° C. and then the reaction mixture is heated to 50 to 150° C. In one embodiment of step a) first the electrochemical oxidation is conducted at temperatures selected from 15 to 75° C., and then the reaction mixture is heated to 75 to 150° C. In one embodiment of step a) first the electrochemical oxidation is conducted at temperatures selected from 15 to 80° C. and then the reaction mixture is heated to 80 to 150° C.

In one embodiment of step a) first the electrochemical oxidation is conducted at temperatures selected from 15 to 80° C. and then the reaction mixture is heated to 80 to 140° C. In one embodiment of step a) first the electrochemical oxidation is conducted at temperatures selected from 15 to 90° C. and then the reaction mixture is heated to 90 to 120° C.

In one embodiment of step a) first the electrochemical oxidation is conducted at temperatures selected from 15 to 150° C., 15 to 120° C., and 20 to 100° C., wherein the temperature can be constant, variable, increasing, decreasing or a specific temperature program can also be run, and then the reaction mixture is heated, wherein the temperature can be constant, variable, increasing, decreasing or a specific temperature program can also be run.

In one embodiment of step a), first the electrochemical oxidation is conducted and then the reaction mixture is heated, wherein the temperature can be constant, variable, increasing, decreasing or a specific temperature program can also be run, and wherein the reaction mixture is heated to temperatures from 75 to 150° C. In one embodiment of step a), first the electrochemical oxidation is conducted and then the reaction mixture is heated, wherein the temperature can be constant, variable, increasing, decreasing or a specific temperature program can also be run, and wherein the reaction mixture is heated to temperatures from 80 to 150° C. In one embodiment of step a), first the electrochemical oxidation is conducted and then the reaction mixture is heated, wherein the temperature can be constant, variable, increasing, decreasing or a specific temperature program can also be run, and wherein the reaction mixture is heated to temperatures from 80 to 140° C. In one embodiment of step a), first the electrochemical oxidation is conducted and then the reaction mixture is heated, wherein the temperature can be constant, variable, increasing, decreasing or a specific temperature program can also be run, and wherein the reaction mixture is heated to temperatures from 90 to 120° C.

In one embodiment of step a) first the electrochemical oxidation is conducted, wherein the temperature can be constant, variable, increasing, decreasing or a specific temperature program can also be run, and then the reaction mixture is heated, wherein the temperature can be constant, variable, increasing, decreasing or a specific temperature program can also be run.

In one embodiment of step a) first the electrochemical oxidation is conducted at temperatures from 15 to 150° C., wherein the temperature can be constant, variable, increasing, decreasing or a specific temperature program can also be run, and then the reaction mixture is heated to temperatures from 50 to 150° C., wherein the temperature can be constant, variable, increasing, decreasing or a specific temperature program can also be run.

In one embodiment of step a)
first the electrochemical oxidation is conducted at temperatures selected from 15 to 150° C., 15 to 120° C., and 20 to 100° C., wherein the temperature can be constant, variable, increasing, decreasing or a specific temperature program can also be run, and
then the reaction mixture is heated to temperatures selected from 50 to 150° C., 75 to 150° C., 80 to 150° C., 80 to 140° C., 90 to 120° C., wherein the temperature can be constant, variable, increasing, decreasing or a specific temperature program can also be run.

In one embodiment of step a)
first the electrochemical oxidation is conducted at temperatures from 15 to 150° C., wherein the temperature can be constant, variable, increasing, decreasing or a specific temperature program can also be run, and
then the reaction mixture is heated to temperatures selected from 50 to 150° C., 75 to 150° C., 80 to 150° C., 80 to 140° C., 90 to 120° C., wherein the temperature can be constant, variable, increasing, decreasing or a specific temperature program can also be run.

In one embodiment of step a)
first the electrochemical oxidation is conducted at temperatures from 15 to 120° C., wherein the temperature can be constant, variable, increasing, decreasing or a specific temperature program can also be run, and
then the reaction mixture is heated to temperatures selected from 50 to 150° C., 75 to 150° C., 80 to 150° C., 80 to 140° C., 90 to 120° C., wherein the temperature can be constant, variable, increasing, decreasing or a specific temperature program can also be run.

In one embodiment of step a)
first the electrochemical oxidation is conducted at temperatures from 20 to 100° C., wherein the temperature can be constant, variable, increasing, decreasing or a specific temperature program can also be run, and
then the reaction mixture is heated to temperatures selected from 50 to 150° C., 75 to 150° C., 80 to 150° C., 80 to 140° C., 90 to 120° C., wherein the temperature can be constant, variable, increasing, decreasing or a specific temperature program can also be run.

In one embodiment of step a)
first the electrochemical oxidation is conducted at temperatures selected from 15 to 150° C., 15 to 120° C., and 20 to 100° C., wherein the temperature can be constant, variable, increasing, decreasing or a specific temperature program can also be run, and
then the reaction mixture is heated to temperatures from 50 to 150° C., wherein the temperature can be constant, variable, increasing, decreasing or a specific temperature program can also be run.

In one embodiment of step a)
first the electrochemical oxidation is conducted at temperatures selected from 15 to 150° C., 15 to 120° C., and 20 to 100° C., wherein the temperature can be constant, variable, increasing, decreasing or a specific temperature program can also be run, and
then the reaction mixture is heated to temperatures from 75 to 150° C., wherein the temperature can be constant, variable, increasing, decreasing or a specific temperature program can also be run.

In one embodiment of step a)
first the electrochemical oxidation is conducted at temperatures selected from 15 to 150° C., 15 to 120° C., and 20 to 100° C., wherein the temperature can be constant, variable, increasing, decreasing or a specific temperature program can also be run, and
then the reaction mixture is heated to temperatures from 80 to 150° C., wherein the temperature can be constant, variable, increasing, decreasing or a specific temperature program can also be run.

In one embodiment of step a)
first the electrochemical oxidation is conducted at temperatures selected from 15 to 150° C., 15 to 120° C., and 20 to 100° C., wherein the temperature can be constant, variable, increasing, decreasing or a specific temperature program can also be run, and
then the reaction mixture is heated to temperatures from 80 to 140° C., wherein the temperature can be constant, variable, increasing, decreasing or a specific temperature program can also be run.

In one embodiment of step a)
first the electrochemical oxidation is conducted at temperatures selected from 15 to 150° C., 15 to 120° C., and 20 to 100° C., wherein the temperature can be constant, variable, increasing, decreasing or a specific temperature program can also be run, and
then the reaction mixture is heated to temperatures from 90 to 120° C., wherein the temperature can be constant, variable, increasing, decreasing or a specific temperature program can also be run.

In one embodiment of step a) a conducting salt is used. In one embodiment of step a) a conducting salt selected from the group of organic ammonia salts, ionic liquids, tetraalkylammonium fluoroborates, quaternary ammonia salts and mixtures thereof is used. In one embodiment of step a) a conducting salt selected from the group of tetraethylammonium tetrafluoroborate, tetrabutylammonium hexafluorophosphate, tetrabutylammonium hydrogen sulfate and mixtures thereof is used.

In one embodiment of step a) the solvent is used in amount for (4R)-4-(4-cyano-2-methoxy-phenyl)-5-ethoxy-2,8-dimethyl-1,4-dihydro-1,6-naphthyridine-3-carboxamide according to formula ent-(I) from 1 to 60 kg/kg. This means that 60 kg solvent is used for 1 kg (4R)-4-(4-cyano-2-methoxy-phenyl)-5-ethoxy-2,8-dimethyl-1,4-dihydro-1,6-naphthyridine-3-carboxamide according to formula ent-(I). In one embodiment of step a) the solvent is used in amount for (4R)-4-(4-cyano-2-methoxy-phenyl)-5-ethoxy-2,8-dimethyl-1,4-dihydro-1,6-naphthyridine-3-carboxamide according to formula ent-(I) from 5 to 50 kg/kg. In one embodiment of step a) the solvent is used in amount for (4R)-4-(4-cyano-2-methoxy-phenyl)-5-ethoxy-2,8-dimethyl-1,4-dihydro-1,6-naphthyridine-3-carboxamide according to formula ent-(I) from 5 to 30 kg/kg. In one embodiment of step a) the solvent is used in an amount from 7 to 25 kg/kg. In one embodiment of step a) the solvent is used in an amount from 8 to 20 kg/kg. In one embodiment of step a) the solvent is used in an amount of 9 to 19 kg/kg.

In one embodiment of step a) the solvent is used in an amount of 80 to 99 wt.-%, based on the amount of the reaction mixture. In one embodiment of step a) the solvent is used in an amount of 85 to 95 wt.-%, based on the amount of the reaction mixture. In one embodiment of step a) the solvent is used in an amount of 85 to 92 wt.-%, based on the amount of the reaction mixture. In one embodiment of step a) the solvent is used in an amount of 90 wt.-%, based on the amount of the reaction mixture.

“wt.-%” is percentage by weight. In one example, if the amount of the reaction mixture is 100 kg, and if a solvent amount of 80 wt.-% is used, the amount of the solvent is 80 kg.

In one embodiment of step a) a solvent is used. In one embodiment of step a) the solvent is selected from aprotic solvents, protic solvent, and mixtures thereof. In one embodiment of step a) the solvent is selected from polar aprotic solvents. In one embodiment of step a) the solvent is selected from the list of polar aprotic solvents: dimethylformamide (DMF), dimethyl sulfoxide, acetonitrile, acetone, dimethylacetamide, N-methyl pyrrolidone, sulfolane or protic solvents like methanol, acetic acid, ethanol, formic acid, n-butanol, propionic acid, methane sulfonic acid, water and mixtures thereof. Further suitable solvents are methoxymethanol, tetramethylurea formamide, DMA (dimethylacetamide), NMP (N-methylpyrrilidone), propionitrile, glycerol, propanol, isopropanol. In one embodiment of step a) the solvent is selected from dimethylformamide (DMF), methanol, acetic acid and mixtures thereof. In one embodiment of step a) the solvent is selected from methanol, acetic acid and mixtures thereof. In one embodiment of step a) the solvent is methanol. In one embodiment of step a) the solvent acetic acid.

Further suitable solvents are acids. Examples of suitable acids are propanoic acid, butyric acid, benzoic acid, p-toluene sulfonic acid, methanesulfonic acid, trifluoroacetic acid, HBF₄, HPF₆, ammonium acetate and mixture of the foregoing.

In one embodiment of step a)
a conducting salt selected from the group of organic ammonia salts, ionic liquids, tetraalkylammonium fluoroborates, quaternary ammonia salts and mixtures thereof; and
a solvent selected from aprotic solvents, protic solvent, and mixtures thereof is used.

In one embodiment of step a)
a conducting salt selected from the group of organic ammonia salts, ionic liquids, tetraalkylammonium fluoroborates, quaternary ammonia salts and mixtures thereof; and
a solvent selected from polar aprotic solvents is used.

In one embodiment of step a)
a conducting salt selected from the group of tetraethylammonium tetrafluoroborate, tetrabutylammonium hexafluorophosphate, tetrabutylammonium hydrogen sulfate and mixtures thereof; and
a solvent selected from aprotic solvents, protic solvent, and mixtures thereof
   is used.

In one embodiment of step a)
a conducting salt selected from the group of tetraethylammonium tetrafluoroborate, tetrabutylammonium hexafluorophosphate, tetrabutylammonium hydrogen sulfate and mixtures thereof; and
a solvent selected from polar aprotic solvents
   is used.

In one embodiment of step a)
a conducting salt selected from the group of organic ammonia salts, ionic liquids, tetraalkylammonium fluoroborates, quaternary ammonia salts and mixtures thereof; and
a polar aprotic solvent selected from dimethylformamide (DMF), dimethyl sulfoxide, acetonitrile, acetone, dimethylacetamide, N-methyl pyrrolidone, sulfolane or protic solvents like methanol, acetic acid, ethanol, formic acid, n-butanol, propionic acid, methane sulfonic acid, water and mixtures thereof

In one embodiment of step a)
a conducting salt selected from the group of tetraethylammonium tetrafluoroborate, tetrabutylammonium hexafluorophosphate, tetrabutylammonium hydrogen sulfate and mixtures thereof; and
a polar aprotic solvent selected from dimethylformamide (DMF), dimethyl sulfoxide, acetonitrile, acetone, dimethylacetamide, N-methyl pyrrolidone, sulfolane or protic solvents like methanol, acetic acid, ethanol, formic acid, n-butanol, propionic acid, methane sulfonic acid, water and mixtures thereof
   is used.

In one embodiment of step a)
a conducting salt selected from the group of organic ammonia salts, ionic liquids, tetraalkylammonium fluoroborates, quaternary ammonia salts and mixtures thereof; and
a solvent is selected from dimethylformamide (DMF), methanol, acetic acid and mixtures thereof
   is used.

In one embodiment of step a)
a conducting salt selected from the group of organic ammonia salts, ionic liquids, tetraalkylammonium fluoroborates, quaternary ammonia salts and mixtures thereof; and
a solvent is selected from dimethylformamide (DMF), methanol, acetic acid and mixtures thereof
   is used.

In one embodiment of step a)
a conducting salt selected from the group of tetraethylammonium tetrafluoroborate, tetrabutylammonium hexafluorophosphate, tetrabutylammonium hydrogen sulfate and mixtures thereof; and
a solvent is selected from dimethylformamide (DMF), methanol, acetic acid and mixtures thereof is used.

In one embodiment of step a)
a conducting salt selected from the group of organic ammonia salts, ionic liquids, tetraalkylammonium fluoroborates, quaternary ammonia salts and mixtures thereof; and
methanol
   is used.

In one embodiment of step a)
a conducting salt selected from the group of organic ammonia salts, ionic liquids tetraalkylammonium fluoroborates, quaternary ammonia salts and mixtures thereof; and
methanol
   is used.

In one embodiment of step a)
a conducting salt selected from the group of tetraethylammonium tetrafluoroborate, tetrabutylammonium hexafluorophosphate, tetrabutylammonium hydrogen sulfate and mixtures thereof; and
methanol
is used.

In one embodiment of step a)
a conducting salt selected from the group of organic ammonia salts, ionic liquids, tetraalkylammonium fluoroborates, quaternary ammonia salts and mixtures thereof; and
acetic acid
   is used.

In one embodiment of step a)
a conducting salt selected from the group of organic ammonia salts, ionic liquids tetraalkylammonium fluoroborates, quaternary ammonia salts and mixtures thereof; and
acetic acid
   is used.

In one embodiment of step a)
a conducting salt selected from the group of tetraethylammonium tetrafluoroborate, tetrabutylammonium hexafluorophosphate, tetrabutylammonium hydrogen sulfate and mixtures thereof; and
acetic acid
   is used.

In one embodiment of step a), 0.05 to 1 equivalents of conducting salt is used, based on 1 equivalent of the compound according to formula ent-(I). This means, if 0.05 equivalent (0.05 mol) of conducting salt is used, 1 equivalent (1 mol) of the compound according to formula ent-(I) is used.

In one embodiment of step a), 0.1 to 0.5 equivalents of conducting salt are used, based on 1 equivalent of the compound according to formula ent-(I). In one embodiment of step a), 0.15 to 0.35 equivalents of conducting salt are used, based on 1 equivalent of the compound according to formula ent-(I). In one embodiment of step a), 0.2 to 0.3 equivalents of conducting salt are used, based on 1 equivalent of the compound according to formula ent-(I).

In one embodiment of step a) an acid is used. In one embodiment of step a) an acid selected from organic acids, organic polar acids and mixtures thereof is used. In one embodiment of step a) an organic acid is used. In one embodiment of step a) an organic acid selected from carboxylic acids and sulfonic acids is used. In one embodiment of step a) an organic polar is used. In one embodiment of step a) an acid selected from acetic acid, formic acid and mixtures thereof is used. In one embodiment of step a) acetic acid.

In one embodiment of step a) the acid is used in an amount of 50 to 500 mmol per L reaction mixture. In one embodiment of step a) the acid is used in an amount of 100 to 300 mmol per L reaction mixture. In one embodiment of step a) the acid is used in an amount of 150 to 250 mmol per L reaction mixture.

In one embodiment of step a) 0.1 to 10 equivalents of acid is used. In one embodiment of step a) 0.1 to 5 equivalents of acid is used. In one embodiment of step a) 0.5 to 3 equivalents of acid is used. In one embodiment of step a) 1.5 to 2 equivalents of acid is used. In one embodiment of step a) 1.7 equivalents of acid is used. The equivalents are based on the compound according to formula ent-(I). This means if 1.7 equivalent (1.7 mol) of acid 1 equivalent (1 mol) of the compound according to formula ent-(I) is used.

In one embodiment of step a) the electrochemical oxidation is carried out at a cell voltage of 0.1 to 20 V. In one embodiment of step a) the electrochemical oxidation is carried out at a cell voltage of 0.1 to 15 V. In one embodiment of step a) the electrochemical oxidation is carried out at a cell voltage of 0.1 to 10 V. In one embodiment of step a) the electrochemical oxidation is carried out at a cell voltage of 0.1 to 5 V.

In one embodiment of step a) the electrochemical oxidation is carried out at a cell voltage of 0.5 to 20 V. In one embodiment of step a) the electrochemical oxidation is carried out at a cell voltage of 0.5 to 15 V. In one embodiment of step a) the electrochemical oxidation is carried out at a cell voltage of 0.5 to 10 V. In one embodiment of step a) the electrochemical oxidation is carried out at a cell voltage of 0.5 to 5 V.

In one embodiment of step a) the electrochemical oxidation is carried out at a cell voltage of 1 to 20 V. In one embodiment of step a) the electrochemical oxidation is carried out at a cell voltage of 1 to 15 V. In one embodiment of step a) the electrochemical oxidation is carried out at a cell voltage of 1 to 10 V. In one embodiment of step a) the electrochemical oxidation is carried out at a cell voltage of 1 to 5 V.

In one embodiment of step a) the electrochemical oxidation is carried out at a cell voltage of 2 to 20 V. In one embodiment of step a) the electrochemical oxidation is carried out at a cell voltage of 2 to 15 V. In one embodiment of step a) the electrochemical oxidation is carried out at a cell voltage of 2 to 10 V. In one embodiment of step a) the electrochemical oxidation is carried out at a cell voltage of 2 to 5 V.

In one embodiment of step a) the electrochemical oxidation is carried out at a cell voltage of 3 to 20 V. In one embodiment of step a) the electrochemical oxidation is carried out at a cell voltage of 3 to 15 V. In one embodiment of step a) the electrochemical oxidation is carried out at a cell voltage of 3 to 10 V. In one embodiment of step a) the electrochemical oxidation is carried out at a cell voltage of 3 to 5 V.

In one embodiment of step a) the electrochemical oxidation is carried out at a cell voltage of less than 20 V. In one embodiment of step a) the electrochemical oxidation is carried out at a cell voltage of less than 19 V. In one embodiment of step a) the electrochemical oxidation is carried out at a cell voltage of less than 18 V. In one embodiment of step a) the electrochemical oxidation is carried out at a cell voltage of less than 17 V. In one embodiment of step a) the electrochemical oxidation is carried out at a cell voltage of less than 16 V. In one embodiment of step a) the electrochemical oxidation is carried out at a cell voltage less than 15V. In one embodiment of step a) the electrochemical oxidation is carried out at a cell voltage of less than 14 V. In one embodiment step a) the electrochemical oxidation is carried out at a cell voltage of less than 13 V. In one embodiment of step a) the electrochemical oxidation is carried out at a cell voltage of less than 12 V. In one embodiment of step a) the electrochemical oxidation is carried out at a cell voltage of less than 11 V. In one embodiment of step a) the electrochemical oxidation is carried out at a cell voltage of less than 10 V. In one embodiment of step a) the electrochemical oxidation is carried out at a cell voltage of less than 20 V, 19 V, 18 V, 17 V, 16 V, 15 V, 14 V, 13 V, 12 V, 11 V, 10 V, 9 V, 8V, 7 V, 6 V, 5 V, 4 V, 3 V, 2 V, 1 V, 0.5 V or 0.1 V.

In one embodiment of step a) the electrochemical oxidation is carried out at current densities from 1 to 5000 A/m². In one embodiment of step a) the electrochemical oxidation is carried out at a current density of 1 to 500 A/m². In one embodiment of step a) the electrochemical oxidation is carried out at current densities of 1 to 100 A/m². In one embodiment of step a) the electrochemical oxidation is carried out at current densities of 1 to 50 A/m². In one embodiment of step a) the electrochemical oxidation is carried out at current densities from 5 to 5000 A/m². In one embodiment of step a) the electrochemical oxidation is carried out at a current density of 5 to 500 A/m². In one embodiment of step a) the electrochemical oxidation is carried out at current densities of 5 to 1000 A/m². In one embodiment of step a) the electrochemical oxidation is carried out at current densities of 5 to 50 A/m². In one embodiment of step a) the electrochemical oxidation is carried out at current densities from 10 to 5000 A/m². In one embodiment of step a) the electrochemical oxidation is carried out at a current density of 10 to 500 A/m². In one embodiment of step a) the electrochemical oxidation is carried out at of a current density of electrochemical oxidation is carried out at current densities of 10 to 100 A/m². In one embodiment of step a) the electrochemical oxidation is carried out at of a current density of electrochemical oxidation is carried out at current densities of 10 to 50 A/m².

In one embodiment of step a) the current density used is constant, variable, increasing, decreasing or a specific temperature program is run.

In one embodiment of step a) the electrochemical oxidation is carried out at current densities from 1 to 5000 A/m², wherein the current density is constant, variable, increasing, decreasing or a specific program is run. In one embodiment of step a) the electrochemical oxidation is carried out at a current density of 1 to 500 A/m², wherein the current density is constant, variable, increasing, decreasing or a specific program is run. In one embodiment of step a) the electrochemical oxidation is carried out at a current density of 1 to 100 A/m², wherein the current density is constant, variable, increasing, decreasing or a specific program is run. In one embodiment of step a) the electrochemical oxidation is carried out at current densities of 1 to 50 A/m², wherein the current density is constant, variable, increasing, decreasing or a specific program is run.

In one embodiment of step a) the electrochemical oxidation is carried out at current densities from 5 to 5000 A/m², wherein the current density is constant, variable, increasing, decreasing or a specific program is run. In one embodiment of step a) the electrochemical oxidation is carried out at a current density of 5 to 500 A/m², wherein the current density is constant, variable, increasing, decreasing or a specific program is run. In one embodiment of step a) the electrochemical oxidation is carried out at a current density of 5 to 100 A/m², wherein the current density is constant, variable, increasing, decreasing or a specific program is run. In one embodiment of step a) the electrochemical oxidation is carried out at current densities of 5 to 50 A/m², wherein the current density is constant, variable, increasing, decreasing or a specific program is run.

In one embodiment of step a) the electrochemical oxidation is carried out at current densities from 10 to 5000 A/m², wherein the current density is constant, variable, increasing, decreasing or a specific program is run. In one embodiment of step a) the electrochemical oxidation is carried out at a current density of 10 to 500 A/m², wherein the current density is constant, variable, increasing, decreasing or a specific program is run. In one embodiment of step a) the electrochemical oxidation is carried out at of a current density of electrochemical oxidation is carried out at current densities of 10 to 100 A/m², wherein the current density is constant, variable, increasing, decreasing or a specific program is run. In one embodiment of step a) the electrochemical oxidation is carried out at of a current density of electrochemical oxidation is carried out at current densities of 10 to 50 A/m², wherein the current density is constant, variable, increasing, decreasing or a specific program is run.

In one embodiment of step a), the current density used is constant. In one embodiment of step a) the electrochemical oxidation is carried out at current densities from 1 to 5000 A/m², wherein the current density used is constant. In one embodiment of step a) the electrochemical oxidation is carried out at a current density of 1 to 500 A/m², wherein the current density used is constant. In one embodiment of step a) the electrochemical oxidation is carried out at current densities of 1 to 100 A/m², wherein the current density used is constant. In one embodiment of step a) the electrochemical oxidation is carried out at current densities of 1 to 50 A/m², wherein the current density used is constant. In one embodiment of step a) the electrochemical oxidation is carried out at current densities from 5 to 5000 A/m², wherein the current density used is constant. In one embodiment of step a) the electrochemical oxidation is carried out at a current density of 5 to 500 A/m², wherein the current density used is constant. In one embodiment of step a) the electrochemical oxidation is carried out at current densities of 5 to 100 A/m², wherein the current density used is constant. In one embodiment of step a) the electrochemical oxidation is carried out at current densities of 5 to 50 A/m², wherein the current density used is constant.

In one embodiment of step a) the electrochemical oxidation is carried out at current densities from 10 to 5000 A/m², wherein the current density used is constant. In one embodiment of step a) the electrochemical oxidation is carried out at a current density of 10 to 500 A/m², wherein the current density used is constant.

In one embodiment of step a) the electrochemical oxidation is carried out at of a current density of electrochemical oxidation is carried out at current densities of 10 to 1000 A/m², wherein the current density used is constant. In one embodiment of step a) the electrochemical oxidation is carried out at of a current density of electrochemical oxidation is carried out at current densities of 10 to 100 A/m², wherein the current density used is constant. In one embodiment of step a) the electrochemical oxidation is carried out at of a current density of electrochemical oxidation is carried out at current densities of 10 to 50 A/m², wherein the current density used is constant.

In one embodiment of step a), the current density used is variable.

In one embodiment of step a) the electrochemical oxidation is carried out at current densities from 1 to 5000 A/m², wherein the current density used is variable. In one embodiment of step a) the electrochemical oxidation is carried out at a current density of 1 to 500 A/m², wherein the current density used is variable. In one embodiment of step a) the electrochemical oxidation is carried out at current densities of 1 to 100 A/m², wherein the current density used is variable. In one embodiment of step a) the electrochemical oxidation is carried out at current densities of 1 to 50 A/m², wherein the current density used is variable. In one embodiment of step a) the electrochemical oxidation is carried out at current densities from 5 to 5000 A/m², wherein the current density used is variable. In one embodiment of step a) the electrochemical oxidation is carried out at a current density of 5 to 500 A/m², wherein the current density used is variable. In one embodiment of step a) the electrochemical oxidation is carried out at current densities of 5 to 100 A/m², wherein the current density used is variable. In one embodiment of step a) the electrochemical oxidation is carried out at current densities of 5 to 50 A/m², wherein the current density used is variable. In one embodiment of step a) the electrochemical oxidation is carried out at current densities from 10 to 5000 A/m², wherein the current density used is variable. In one embodiment of step a) the electrochemical oxidation is carried out at a current density of 10 to 500 A/m², wherein the current density used is variable. In one embodiment of step a) the electrochemical oxidation is carried out at of a current density of electrochemical oxidation is carried out at current densities of 10 to 100 A/m², wherein the current density used is variable. In one embodiment of step a) the electrochemical oxidation is carried out at of a current density of electrochemical oxidation is carried out at current densities of 10 to 50 A/m², wherein the current density used is variable.

In one embodiment of step a), the current density used is increased.

In one embodiment of step a) the electrochemical oxidation is carried out at current densities from 1 to 5000 A/m², wherein the current density used is increased. In one embodiment of step a) the electrochemical oxidation is carried out at a current density of 1 to 500 A/m², wherein the current density used is increased. In one embodiment of step a) the electrochemical oxidation is carried out at current densities of 1 to 100 A/m², wherein the current density used is increased. In one embodiment of step a) the electrochemical oxidation is carried out at current densities of 1 to 50 A/m², wherein the current density used is increased. In one embodiment of step a) the electrochemical oxidation is carried out at current densities from 5 to 5000 A/m², wherein the current density used is increased. In one embodiment of step a) the electrochemical oxidation is carried out at a current density of 5 to 500 A/m², wherein the current density used is increased. In one embodiment of step a) the electrochemical oxidation is carried out at current densities of 5 to 100 A/m², wherein the current density used is increased. In one embodiment of step a) the electrochemical oxidation is carried out at current densities of 5 to 50 A/m², wherein the current density used is increased. In one embodiment of step a) the electrochemical oxidation is carried out at current densities from 10 to 5000 A/m², wherein the current density used is increased. In one embodiment of step a) the electrochemical oxidation is carried out at a current density of 10 to 500 A/m², wherein the current density used is increased. In one embodiment of step a) the electrochemical oxidation is carried out at of a current density of electrochemical oxidation is carried out at current densities of 10 to 100 A/m², wherein the current density used is increased. In one embodiment of step a) the electrochemical oxidation is carried out at of a current density of electrochemical oxidation is carried out at current densities of 10 to 50 A/m², wherein the current density used is increased.

In one embodiment of step a), the current density used is decreased.

In one embodiment of step a), the current density used is stepwise decreased. Examples of the use of stepwise currency decrease are exemplarily described in the experimental part below. Examples can be found in section C.1.1. below. In one embodiment of step a), the current density used is exponentially decreased. In one embodiment of step a), the current density used is stepwise decreased, wherein each step can be identical or varying. An example of a decrease or stepwise decrease with an identical step is that the step is always the same, for example always by the identical amount of current density is used. An example of this would be that the step is in increments of 10 A/m². An example of a decrease or stepwise decrease with varying steps is that in the first step 10 A/m² is used, then in the next step 1 A/m² is used, and so on.

The skilled person would not have expected that a one-pot synthesis would be successful as it was already described in U.S. Ser. No. 15/753,406 A1 that direct electrochemical oxidation (i.e. without mediator) failed (see example 28 of U.S. Ser. No. 15/753,406 A1, with <50% yield for XVII). This problem was overcome by stepwise current decrease and, thus, high current und chemical selectivity can be reached. The oxidation selectivity is surprisingly so high that no secondary components were raised up, which can interfere with the racemization and reduction step and, thus, no isolation and purge after each step would be required. It was surprising that by stepwise current decrease a high current and chemical selectivity can be reached. The oxidation selectivity was surprisingly so high that no secondary components were raised up. Such secondary components can interfere with the following racemization and reduction step and would require an isolation and purge after each step.

In one embodiment of step a) the electrochemical oxidation is carried out at current densities from 1 to 5000 A/m², wherein the current density used is decreased. In one embodiment of step a) the electrochemical oxidation is carried out at current densities from 1 to 500 A/m², wherein the current density used is decreased. In one embodiment of step a) the electrochemical oxidation is carried out at current densities from 1 to 100 A/m², wherein the current density used is decreased. In one embodiment of step a) the electrochemical oxidation is carried out at current densities from 1 to 50 A/m², wherein the current density used is decreased.

In one embodiment of step a) the electrochemical oxidation is carried out at current densities from 5 to 5000 A/m², wherein the current density used is decreased. In one embodiment of step a) the electrochemical oxidation is carried out at current densities from 5 to 500 A/m², wherein the current density used is decreased. In one embodiment of step a) the electrochemical oxidation is carried out at current densities from 5 to 100 A/m², wherein the current density used is decreased. In one embodiment of step a) the electrochemical oxidation is carried out at current densities from 5 to 50 A/m², wherein the current density used is decreased.

In one embodiment of step a) the electrochemical oxidation is carried out at current densities from 10 to 5000 A/m², wherein the current density used is decreased. In one embodiment of step a) the electrochemical oxidation is carried out at current densities from 10 to 500 A/m², wherein the current density used is decreased. In one embodiment of step a) the electrochemical oxidation is carried out at current densities from 10 to 100 A/m², wherein the current density used is decreased. In one embodiment of step a) the electrochemical oxidation is carried out at current densities from 10 to 50 A/m², wherein the current density used is decreased.

In one embodiment of step a) the electrochemical oxidation is carried out at current densities from 1 to 5000 A/m², wherein the current density used is stepwise decreased. In one embodiment of step a) the electrochemical oxidation is carried out at current densities from 1 to 500 A/m², wherein the current density used is stepwise decreased. In one embodiment of step a) the electrochemical oxidation is carried out at current densities from 1 to 100 A/m², wherein the current density used is stepwise decreased. In one embodiment of step a) the electrochemical oxidation is carried out at current densities from 1 to 50 A/m², wherein the current density used is stepwise decreased.

In one embodiment of step a) the electrochemical oxidation is carried out at current densities from 5 to 5000 A/m², wherein the current density used is stepwise decreased. In one embodiment of step a) the electrochemical oxidation is carried out at current densities from 5 to 500 A/m², wherein the current density used is stepwise decreased. In one embodiment of step a) the electrochemical oxidation is carried out at current densities from 5 to 100 A/m², wherein the current density used is stepwise decreased. In one embodiment of step a) the electrochemical oxidation is carried out at current densities from 5 to 50 A/m², wherein the current density used is stepwise decreased.

In one embodiment of step a) the electrochemical oxidation is carried out at current densities from 10 to 5000 A/m², wherein the current density used is stepwise decreased. In one embodiment of step a) the electrochemical oxidation is carried out at current densities from 10 to 500 A/m², wherein the current density used is stepwise decreased. In one embodiment of step a) the electrochemical oxidation is carried out at current densities from 10 to 100 A/m², wherein the current density used is stepwise decreased. In one embodiment of step a) the electrochemical oxidation is carried out at current densities from 10 to 50 A/m², wherein the current density used is stepwise decreased.

In one embodiment of step a) the electrochemical oxidation is carried out at current densities from 1 to 5000 A/m², wherein the current density used is exponentially decreased. In one embodiment of step a) the electrochemical oxidation is carried out at current densities from 1 to 500 A/m², wherein the current density used is exponentially decreased. In one embodiment of step a) the electrochemical oxidation is carried out at current densities from 1 to 100 A/m², wherein the current density used is exponentially decreased. In one embodiment of step a) the electrochemical oxidation is carried out at current densities from 1 to 50 A/m², wherein the current density used is exponentially decreased.

In one embodiment of step a) the electrochemical oxidation is carried out at current densities from 5 to 5000 A/m², wherein the current density used is exponentially decreased. In one embodiment of step a) the electrochemical oxidation is carried out at current densities from 5 to 500 A/m², wherein the current density used is exponentially decreased. In one embodiment of step a) the electrochemical oxidation is carried out at current densities from 5 to 100 A/m², wherein the current density used is exponentially decreased. In one embodiment of step a) the electrochemical oxidation is carried out at current densities from 5 to 50 A/m², wherein the current density used is exponentially decreased.

In one embodiment of step a) the electrochemical oxidation is carried out at current densities from 10 to 5000 A/m², wherein the current density used is exponentially decreased. In one embodiment of step a) the electrochemical oxidation is carried out at current densities from 10 to 500 A/m², wherein the current density used is exponentially decreased. In one embodiment of step a) the electrochemical oxidation is carried out at current densities from 10 to 100 A/m², wherein the current density used is exponentially decreased. In one embodiment of step a) the electrochemical oxidation is carried out at current densities from 10 to 50 A/m², wherein the current density used is exponentially decreased.

In one embodiment of step a) the electrochemical oxidation is carried out at current densities from 1 to 5000 A/m², wherein the current density used is stepwise decreased in steps of 0.1 to 100 A/m². In one embodiment of step a) the electrochemical oxidation is carried out at current densities from 1 to 500 A/m², wherein the current density used is stepwise decreased in steps of 0.1 to 100 A/m². In one embodiment of step a) the electrochemical oxidation is carried out at current densities from 1 to 100 A/m², wherein the current density used is stepwise decreased in steps of 0.1 to 100 A/m². In one embodiment of step a) the electrochemical oxidation is carried out at current densities from 1 to 50 A/m², wherein the current density used is stepwise decreased in steps of 0.1 to 100 A/m².

In one embodiment of step a) the electrochemical oxidation is carried out at current densities from 5 to 5000 A/m², wherein the current density used is stepwise decreased in steps of 0.1 to 100 A/m². In one embodiment of step a) the electrochemical oxidation is carried out at current densities from 5 to 500 A/m², wherein the current density used is stepwise decreased in steps of 0.1 to 100 A/m². In one embodiment of step a) the electrochemical oxidation is carried out at current densities from 5 to 100 A/m², wherein the current density used is stepwise decreased in steps of 0.1 to 100 A/m². In one embodiment of step a) the electrochemical oxidation is carried out at current densities from 5 to 50 A/m², wherein the current density used is stepwise decreased in steps of 0.1 to 100 A/m².

In one embodiment of step a) the electrochemical oxidation is carried out at current densities from 10 to 5000 A/m², wherein the current density used is stepwise decreased in steps of 0.1 to 100 A/m². In one embodiment of step a) the electrochemical oxidation is carried out at current densities from 10 to 500 A/m², wherein the current density used is stepwise decreased in steps of 0.1 to 100 A/m². In one embodiment of step a) the electrochemical oxidation is carried out at current densities from 10 to 100 A/m², wherein the current density used is stepwise decreased in steps of 0.1 to 100 A/m². In one embodiment of step a) the electrochemical oxidation is carried out at current densities from 10 to 50 A/m², wherein the current density used is stepwise decreased in steps of 0.1 to 100 A/m².

In one embodiment of step a) the electrochemical oxidation is carried out at current densities from 1 to 5000 A/m², wherein the current density used is stepwise decreased in steps of 0.5 to 50 A/m². In one embodiment of step a) the electrochemical oxidation is carried out at current densities from 1 to 500 A/m², wherein the current density used is stepwise decreased in steps of 0.5 to 50 A/m². In one embodiment of step a) the electrochemical oxidation is carried out at current densities from 1 to 50 A/m², wherein the current density used is stepwise decreased in steps of 0.5 to 100 A/m². In one embodiment of step a) the electrochemical oxidation is carried out at current densities from 1 to 50 A/m², wherein the current density used is stepwise decreased in steps of 0.5 to 50 A/m².

In one embodiment of step a) the electrochemical oxidation is carried out at current densities from 5 to 5000 A/m², wherein the current density used is stepwise decreased in steps of 0.5 to 50 A/m². In one embodiment of step a) the electrochemical oxidation is carried out at current densities from 5 to 500 A/m², wherein the current density used is stepwise decreased in steps of 0.5 to 50 A/m². In one embodiment of step a) the electrochemical oxidation is carried out at current densities from 5 to 100 A/m², wherein the current density used is stepwise decreased in steps of 0.5 to 50 A/m². In one embodiment of step a) the electrochemical oxidation is carried out at current densities from 5 to 50 A/m², wherein the current density used is stepwise decreased in steps of 0.5 to 50 A/m².

In one embodiment of step a) the electrochemical oxidation is carried out at current densities from 10 to 5000 A/m², wherein the current density used is stepwise decreased in steps of 0.5 to 50 A/m². In one embodiment of step a) the electrochemical oxidation is carried out at current densities from 10 to 500 A/m², wherein the current density used is stepwise decreased in steps of 0.5 to 50 A/m². In one embodiment of step a) the electrochemical oxidation is carried out at current densities from 10 to 100 A/m², wherein the current density used is stepwise decreased in steps of 0.5 to 50 A/m². In one embodiment of step a) the electrochemical oxidation is carried out at current densities from 10 to 50 A/m², wherein the current density used is stepwise decreased in steps of 0.5 to 50 A/m².

In one embodiment of step a) the electrochemical oxidation is carried out at current densities from 1 to 5000 A/m², wherein the current density used is stepwise decreased in steps of 1 to 10 A/m². In one embodiment of step a) the electrochemical oxidation is carried out at current densities from 1 to 500 A/m², wherein the current density used is stepwise decreased in steps of 1 to 10 A/m². In one embodiment of step a) the electrochemical oxidation is carried out at current densities from 1 to 100 A/m², wherein the current density used is stepwise decreased in steps of 1 to 10 A/m². In one embodiment of step a) the electrochemical oxidation is carried out at current densities from 1 to 50 A/m², wherein the current density used is stepwise decreased in steps of 1 to 10 A/m².

In one embodiment of step a) the electrochemical oxidation is carried out at current densities from 5 to 5000 A/m², wherein the current density used is stepwise decreased in steps of 1 to 10 A/m². In one embodiment of step a) the electrochemical oxidation is carried out at current densities from 5 to 500 A/m², wherein the current density used is stepwise decreased in steps of 1 to 10 A/m². In one embodiment of step a) the electrochemical oxidation is carried out at current densities from 5 to 100 A/m², wherein the current density used is stepwise decreased in steps of 1 to 10 A/m². In one embodiment of step a) the electrochemical oxidation is carried out at current densities from 5 to 50 A/m², wherein the current density used is stepwise decreased in steps of 1 to 10 A/m².

In one embodiment of step a) the electrochemical oxidation is carried out at current densities from 10 to 5000 A/m², wherein the current density used is stepwise decreased in steps of 1 to 10 A/m². In one embodiment of step a) the electrochemical oxidation is carried out at current densities from 10 to 500 A/m², wherein the current density used is stepwise decreased in steps of 1 to 10 A/m². In one embodiment of step a) the electrochemical oxidation is carried out at current densities from 10 to 100 A/m², wherein the current density used is stepwise decreased in steps of 1 to 10 A/m². In one embodiment of step a) the electrochemical oxidation is carried out at current densities from 10 to 50 A/m², wherein the current density used is stepwise decreased in steps of 1 to 10 A/m².

In one embodiment of step a) the electrochemical oxidation is carried out at current densities from 1 to 5000 A/m², wherein the current density used is stepwise decreased in steps of 1 to 5 A/m². In one embodiment of step a) the electrochemical oxidation is carried out at current densities from 1 to 500 A/m², wherein the current density used is stepwise decreased in steps of 1 to 5 A/m². In one embodiment of step a) the electrochemical oxidation is carried out at current densities from 1 to 100 A/m², wherein the current density used is stepwise decreased in steps of 1 to 5 A/m². In one embodiment of step a) the electrochemical oxidation is carried out at current densities from 1 to 50 A/m², wherein the current density used is stepwise decreased in steps of 1 to 5 A/m².

In one embodiment of step a) the electrochemical oxidation is carried out at current densities from 5 to 5000 A/m², wherein the current density used is stepwise decreased in steps of 1 to 5 A/m². In one embodiment of step a) the electrochemical oxidation is carried out at current densities from 5 to 500 A/m², wherein the current density used is stepwise decreased in steps of 1 to 5 A/m². In one embodiment of step a) the electrochemical oxidation is carried out at current densities from 5 to 100 A/m², wherein the current density used is stepwise decreased in steps of 1 to 5 A/m². In one embodiment of step a) the electrochemical oxidation is carried out at current densities from 5 to 50 A/m², wherein the current density used is stepwise decreased in steps of 1 to 5 A/m².

In one embodiment of step a) the electrochemical oxidation is carried out at current densities from 10 to 5000 A/m², wherein the current density used is stepwise decreased in steps of 1 to 5 A/m². In one embodiment of step a) the electrochemical oxidation is carried out at current densities from 10 to 500 A/m², wherein the current density used is stepwise decreased in steps of 1 to 5 A/m². In one embodiment of step a) the electrochemical oxidation is carried out at current densities from 10 to 100 A/m², wherein the current density used is stepwise decreased in steps of 1 to 5 A/m². In one embodiment of step a) the electrochemical oxidation is carried out at current densities from 10 to 50 A/m², wherein the current density used is stepwise decreased in steps of 1 to 5 A/m².

In one embodiment of step a) the electrochemical oxidation is carried out at current densities from 1 to 5000 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m². In one embodiment of step a) the electrochemical oxidation is carried out at current densities from 1 to 500 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m². In one embodiment of step a) the electrochemical oxidation is carried out at current densities from 1 to 100 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m². In one embodiment of step a) the electrochemical oxidation is carried out at current densities from 1 to 50 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m².

In one embodiment of step a) the electrochemical oxidation is carried out at current densities from 5 to 5000 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m². In one embodiment of step a) the electrochemical oxidation is carried out at current densities from 5 to 500 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m². In one embodiment of step a) the electrochemical oxidation is carried out at current densities from 5 to 100 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m². In one embodiment of step a) the electrochemical oxidation is carried out at current densities from 5 to 50 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m².

In one embodiment of step a) the electrochemical oxidation is carried out at current densities from 10 to 5000 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m². In one embodiment of step a) the electrochemical oxidation is carried out at current densities from 10 to 500 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m². In one embodiment of step a) the electrochemical oxidation is carried out at current densities from 10 to 100 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m². In one embodiment of step a) the electrochemical oxidation is carried out at current densities from 10 to 50 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m².

In one embodiment of step a) the electrochemical oxidation is carried out at current densities from 1 to 5000 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are identical. In one embodiment of step a) the electrochemical oxidation is carried out at current densities from 1 to 500 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are identical. In one embodiment of step a) the electrochemical oxidation is carried out at current densities from 1 to 100 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are identical. In one embodiment of step a) the electrochemical oxidation is carried out at current densities from 1 to 50 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are identical.

In one embodiment of step a) the electrochemical oxidation is carried out at current densities from 5 to 5000 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are identical. In one embodiment of step a) the electrochemical oxidation is carried out at current densities from 5 to 500 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are identical. In one embodiment of step a) the electrochemical oxidation is carried out at current densities from 5 to 100 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are identical.

In one embodiment of step a) the electrochemical oxidation is carried out at current densities from 5 to 50 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are identical. In one embodiment of step a) the electrochemical oxidation is carried out at current densities from 10 to 5000 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are identical. In one embodiment of step a) the electrochemical oxidation is carried out at current densities from 10 to 500 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are identical. In one embodiment of step a) the electrochemical oxidation is carried out at current densities from 10 to 100 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are identical. In one embodiment of step a) the electrochemical oxidation is carried out at current densities from 10 to 50 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are identical.

In one embodiment of step a) the electrochemical oxidation is carried out at current densities from 1 to 5000 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are varying. In one embodiment of step a) the electrochemical oxidation is carried out at current densities from 1 to 500 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are varying. In one embodiment of step a) the electrochemical oxidation is carried out at current densities from 1 to 100 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are varying. In one embodiment of step a) the electrochemical oxidation is carried out at current densities from 1 to 50 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are varying.

In one embodiment of step a) the electrochemical oxidation is carried out at current densities from 5 to 5000 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are varying. In one embodiment of step a) the electrochemical oxidation is carried out at current densities from 5 to 500 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are varying. In one embodiment of step a) the electrochemical oxidation is carried out at current densities from 5 to 100 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are varying. In one embodiment of step a) the electrochemical oxidation is carried out at current densities from 5 to 50 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are varying.

In one embodiment of step a) the electrochemical oxidation is carried out at current densities from 10 to 5000 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are varying. In one embodiment of step a) the electrochemical oxidation is carried out at current densities from 10 to 500 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are varying. In one embodiment of step a) the electrochemical oxidation is carried out at current densities from 10 to 100 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are varying. In one embodiment of step a) the electrochemical oxidation is carried out at current densities from 10 to 50 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are varying.

In one embodiment of step a) the electrochemical oxidation is carried out at current densities from 10 to 50 A/m², wherein the current density used is stepwise decreased as follows: from 50 to 40 A/m², then from 40 to 30 A/m², then from 30 to 20 A/m², then from 20 to 10 A/m².

In one embodiment of the sequential one-pot synthesis, the current density used in step a) is lower than the current density used in step b). The selectivity with which compound (XVII) is obtained in this embodiment was surprising. The oxidation selectivity was surprisingly so high that no secondary components were raised up. The oxidation selectivity was surprisingly so high that no secondary components were raised up. Such secondary components can interfere with the following racemization and reduction step and would require an isolation and purge after each step.

In one embodiment of step a) the electrochemical oxidation is carried out at current densities from 1 to 5000 A/m², wherein the current density used in step a) is lower than the current density used in step b). In one embodiment of step a) the electrochemical oxidation is carried out at current densities from 1 to 500 A/m², wherein the current density used in step a) is lower than the current density used in step b). In one embodiment of step a) the electrochemical oxidation is carried out at current densities from 1 to 100 A/m², wherein the current density used in step a) is lower than the current density used in step b). In one embodiment of step a) the electrochemical oxidation is carried out at current densities from 1 to 50 A/m², wherein the current density used in step a) is lower than the current density used in step b).

In one embodiment of step a) the electrochemical oxidation is carried out at current densities from 5 to 5000 A/m², wherein the current density used in step a) is lower than the current density used in step b). In one embodiment of step a) the electrochemical oxidation is carried out at current densities from 5 to 500 A/m², wherein the current density used in step a) is lower than the current density used in step b). In one embodiment of step a) the electrochemical oxidation is carried out at current densities from 5 to 100 A/m², wherein the current density used in step a) is lower than the current density used in step b). In one embodiment of step a) the electrochemical oxidation is carried out at current densities from 5 to 50 A/m², wherein the current density used in step a) is lower than the current density used in step b).

In one embodiment of step a) the electrochemical oxidation is carried out at current densities from 10 to 5000 A/m², wherein the current density used in step a) is lower than the current density used in step b). In one embodiment of step a) the electrochemical oxidation is carried out at current densities from 10 to 500 A/m², wherein the current density used in step a) is lower than the current density used in step b). In one embodiment of step a) the electrochemical oxidation is carried out at current densities from 10 to 100 A/m², wherein the current density used in step a) is lower than the current density used in step b). In one embodiment of step a) the electrochemical oxidation is carried out at current densities from 10 to 50 A/m², wherein the current density used in step a) is lower than the current density used in step b).

In one embodiment, the current density used in oxidation in step a) is lower than the current density used in the electrochemical reduction step b) and the current density used in step a) is decreased. It was surprising that by current decrease a high current and chemical selectivity can be reached. The oxidation selectivity was surprisingly so high that no secondary components were raised up. Such components can interfere with the racemization and reduction step and thus no isolation and purge after each step would be required.

In one embodiment of step a) the electrochemical oxidation is carried out at current densities from 1 to 5000 A/m², wherein the current density used in step a) is decreased, and wherein the current density used in step a) is lower than the current density used in step b). In one embodiment of step a) the electrochemical oxidation is carried out at current densities from 1 to 500 A/m², wherein the current density used in step a) is decreased, and wherein the current density used in step a) is lower than the current density used in step b). In one embodiment of step a) the electrochemical oxidation is carried out at current densities from 1 to 100 A/m², wherein the current density used in step a) is decreased, and wherein the current density used in step a) is lower than the current density used in step b). In one embodiment of step a) the electrochemical oxidation is carried out at current densities from 1 to 50 A/m², wherein the current density used in step a) is decreased, and wherein the current density used in step a) is lower than the current density used in step b).

In one embodiment of step a) the electrochemical oxidation is carried out at current densities from 5 to 5000 A/m², wherein the current density used in step a) is decreased, and wherein the current density used in step a) is lower than the current density used in step b). In one embodiment of step a) the electrochemical oxidation is carried out at current densities from 5 to 500 A/m², wherein the current density used in step a) is decreased, and wherein the current density used in step a) is lower than the current density used in step b). In one embodiment of step a) the electrochemical oxidation is carried out at current densities from 5 to 100 A/m², wherein the current density used in step a) is decreased, and wherein the current density used in step a) is lower than the current density used in step b). In one embodiment of step a) the electrochemical oxidation is carried out at current densities from 5 to 50 A/m², wherein the current density used in step a) is decreased, and wherein the current density used in step a) is lower than the current density used in step b).

In one embodiment of step a) the electrochemical oxidation is carried out at current densities from 10 to 5000 A/m², wherein the current density used in step a) is decreased, and wherein the current density used in step a) is lower than the current density used in step b). In one embodiment of step a) the electrochemical oxidation is carried out at current densities from 10 to 500 A/m², wherein the current density used in step a) is decreased, and wherein the current density used in step a) is lower than the current density used in step b). In one embodiment of step a) the electrochemical oxidation is carried out at current densities from 10 to 100 A/m², wherein the current density used in step a) is decreased, and wherein the current density used in step a) is lower than the current density used in step b). In one embodiment of step a) the electrochemical oxidation is carried out at current densities from 10 to 50 A/m², wherein the current density used in step a) is decreased, and wherein the current density used in step a) is lower than the current density used in step b).

In one embodiment, the current density used in oxidation in step a) is lower than the current density used in the electrochemical reduction step b), and the current density used in step a) is stepwise decreased. It was surprising that by current stepwise decrease a high current and chemical selectivity was reached. The oxidation selectivity was surprisingly so high that no secondary components were raised up. Such components interfere with the racemization and reduction step and thus no isolation and purge after each step is required.

In one embodiment of step a) the electrochemical oxidation is carried out at current densities from 1 to 5000 A/m², wherein the current density used in step a) is stepwise decreased, and wherein the current density used in step a) is lower than the current density used in step b). In one embodiment of step a) the electrochemical oxidation is carried out at current densities from 1 to 500 A/m², wherein the current density used in step a) is stepwise decreased, and wherein the current density used in step a) is lower than the current density used in step b). In one embodiment of step a) the electrochemical oxidation is carried out at current densities from 1 to 100 A/m², wherein the current density used in step a) is stepwise decreased, and wherein the current density used in step a) is lower than the current density used in step b). In one embodiment of step a) the electrochemical oxidation is carried out at current densities from 1 to 50 A/m², wherein the current density used in step a) is stepwise decreased, and wherein the current density used in step a) is lower than the current density used in step b).

In one embodiment of step a) the electrochemical oxidation is carried out at current densities from 5 to 5000 A/m², wherein the current density used in step a) is stepwise decreased, and wherein the current density used in step a) is lower than the current density used in step b). In one embodiment of step a) the electrochemical oxidation is carried out at current densities from 5 to 500 A/m², wherein the current density used in step a) is stepwise decreased, and wherein the current density used in step a) is lower than the current density used in step b). In one embodiment of step a) the electrochemical oxidation is carried out at current densities from 5 to 100 A/m², wherein the current density used in step a) is stepwise decreased, and wherein the current density used in step a) is lower than the current density used in step b). In one embodiment of step a) the electrochemical oxidation is carried out at current densities from 5 to 50 A/m², wherein the current density used in step a) is stepwise decreased, and wherein the current density used in step a) is lower than the current density used in step b).

In one embodiment of step a) the electrochemical oxidation is carried out at current densities from 10 to 5000 A/m², wherein the current density used in step a) is stepwise decreased, and wherein the current density used in step a) is lower than the current density used in step b). In one embodiment of step a) the electrochemical oxidation is carried out at current densities from 10 to 500 A/m², wherein the current density used in step a) is stepwise decreased, and wherein the current density used in step a) is lower than the current density used in step b). In one embodiment of step a) the electrochemical oxidation is carried out at current densities from 10 to 100 A/m², wherein the current density used in step a) is stepwise decreased, and wherein the current density used in step a) is lower than the current density used in step b). In one embodiment of step a) the electrochemical oxidation is carried out at current densities from 10 to 50 A/m², wherein the current density used in step a) is stepwise decreased, and wherein the current density used in step a) is lower than the current density used in step b).

In one embodiment of step a) the electrochemical oxidation is carried out at current densities from 1 to 5000 A/m², wherein the current density used is stepwise decreased in steps of 0.1 to 100 A/m², wherein the current density used in step a) is lower than the current density used in step b). In one embodiment of step a) the electrochemical oxidation is carried out at current densities from 1 to 500 A/m², wherein the current density used is stepwise decreased in steps of 0.1 to 100 A/m², wherein the current density used in step a) is lower than the current density used in step b). In one embodiment of step a) the electrochemical oxidation is carried out at current densities from 1 to 100 A/m², wherein the current density used is stepwise decreased in steps of 0.1 to 100 A/m², wherein the current density used in step a) is lower than the current density used in step b). In one embodiment of step a) the electrochemical oxidation is carried out at current densities from 1 to 50 A/m², wherein the current density used is stepwise decreased in steps of 0.1 to 100 A/m², wherein the current density used in step a) is lower than the current density used in step b).

In one embodiment of step a) the electrochemical oxidation is carried out at current densities from 5 to 5000 A/m², wherein the current density used is stepwise decreased in steps of 0.1 to 100 A/m², wherein the current density used in step a) is lower than the current density used in step b). In one embodiment of step a) the electrochemical oxidation is carried out at current densities from 5 to 500 A/m², wherein the current density used is stepwise decreased in steps of 0.1 to 100 A/m², wherein the current density used in step a) is lower than the current density used in step b). In one embodiment of step a) the electrochemical oxidation is carried out at current densities from 5 to 100 A/m², wherein the current density used is stepwise decreased in steps of 0.1 to 100 A/m², wherein the current density used in step a) is lower than the current density used in step b). In one embodiment of step a) the electrochemical oxidation is carried out at current densities from 5 to 50 A/m², wherein the current density used is stepwise decreased in steps of 0.1 to 100 A/m², wherein the current density used in step a) is lower than the current density used in step b).

In one embodiment of step a) the electrochemical oxidation is carried out at current densities from 10 to 5000 A/m², wherein the current density used is stepwise decreased in steps of 0.1 to 100 A/m², wherein the current density used in step a) is lower than the current density used in step b). In one embodiment of step a) the electrochemical oxidation is carried out at current densities from 10 to 500 A/m², wherein the current density used is stepwise decreased in steps of 0.1 to 100 A/m², wherein the current density used in step a) is lower than the current density used in step b). In one embodiment of step a) the electrochemical oxidation is carried out at current densities from 10 to 100 A/m², wherein the current density used is stepwise decreased in steps of 0.1 to 100 A/m², wherein the current density used in step a) is lower than the current density used in step b). In one embodiment of step a) the electrochemical oxidation is carried out at current densities from 10 to 50 A/m², wherein the current density used is stepwise decreased in steps of 0.1 to 100 A/m², wherein the current density used in step a) is lower than the current density used in step b).

In one embodiment of step a) the electrochemical oxidation is carried out at current densities from 1 to 5000 A/m², wherein the current density used is stepwise decreased in steps of 0.5 to 50 A/m², wherein the current density used in step a) is lower than the current density used in step b). In one embodiment of step a) the electrochemical oxidation is carried out at current densities from 1 to 500 A/m², wherein the current density used is stepwise decreased in steps of 0.5 to 50 A/m², wherein the current density used in step a) is lower than the current density used in step b). In one embodiment of step a) the electrochemical oxidation is carried out at current densities from 1 to 50 A/m², wherein the current density used is stepwise decreased in steps of 0.5 to 100 A/m², wherein the current density used in step a) is lower than the current density used in step b). In one embodiment of step a) the electrochemical oxidation is carried out at current densities from 1 to 50 A/m², wherein the current density used is stepwise decreased in steps of 0.5 to 50 A/m², wherein the current density used in step a) is lower than the current density used in step b).

In one embodiment of step a) the electrochemical oxidation is carried out at current densities from 5 to 5000 A/m², wherein the current density used is stepwise decreased in steps of 0.5 to 50 A/m², wherein the current density used in step a) is lower than the current density used in step b). In one embodiment of step a) the electrochemical oxidation is carried out at current densities from 5 to 500 A/m², wherein the current density used is stepwise decreased in steps of 0.5 to 50 A/m², wherein the current density used in step a) is lower than the current density used in step b). In one embodiment of step a) the electrochemical oxidation is carried out at current densities from 5 to 100 A/m², wherein the current density used is stepwise decreased in steps of 0.5 to 50 A/m², wherein the current density used in step a) is lower than the current density used in step b). In one embodiment of step a) the electrochemical oxidation is carried out at current densities from 5 to 50 A/m², wherein the current density used is stepwise decreased in steps of 0.5 to 50 A/m², wherein the current density used in step a) is lower than the current density used in step b).

In one embodiment of step a) the electrochemical oxidation is carried out at current densities from 10 to 5000 A/m², wherein the current density used is stepwise decreased in steps of 0.5 to 50 A/m², wherein the current density used in step a) is lower than the current density used in step b). In one embodiment of step a) the electrochemical oxidation is carried out at current densities from 10 to 500 A/m², wherein the current density used is stepwise decreased in steps of 0.5 to 50 A/m², wherein the current density used in step a) is lower than the current density used in step b). In one embodiment of step a) the electrochemical oxidation is carried out at current densities from 10 to 100 A/m², wherein the current density used is stepwise decreased in steps of 0.5 to 50 A/m², wherein the current density used in step a) is lower than the current density used in step b). In one embodiment of step a) the electrochemical oxidation is carried out at current densities from 10 to 50 A/m², wherein the current density used is stepwise decreased in steps of 0.5 to 50 A/m², wherein the current density used in step a) is lower than the current density used in step b).

In one embodiment of step a) the electrochemical oxidation is carried out at current densities from 1 to 5000 A/m², wherein the current density used is stepwise decreased in steps of 1 to 10 A/m², wherein the current density used in step a) is lower than the current density used in step b). In one embodiment of step a) the electrochemical oxidation is carried out at current densities from 1 to 500 A/m², wherein the current density used is stepwise decreased in steps of 1 to 10 A/m², wherein the current density used in step a) is lower than the current density used in step b). In one embodiment of step a) the electrochemical oxidation is carried out at current densities from 1 to 100 A/m², wherein the current density used is stepwise decreased in steps of 1 to 10 A/m², wherein the current density used in step a) is lower than the current density used in step b). In one embodiment of step a) the electrochemical oxidation is carried out at current densities from 1 to 50 A/m², wherein the current density used is stepwise decreased in steps of 1 to 10 A/m², wherein the current density used in step a) is lower than the current density used in step b).

In one embodiment of step a) the electrochemical oxidation is carried out at current densities from 5 to 5000 A/m², wherein the current density used is stepwise decreased in steps of 1 to 10 A/m², wherein the current density used in step a) is lower than the current density used in step b). In one embodiment of step a) the electrochemical oxidation is carried out at current densities from 5 to 500 A/m², wherein the current density used is stepwise decreased in steps of 1 to 10 A/m², wherein the current density used in step a) is lower than the current density used in step b). In one embodiment of step a) the electrochemical oxidation is carried out at current densities from 5 to 100 A/m², wherein the current density used is stepwise decreased in steps of 1 to 10 A/m², wherein the current density used in step a) is lower than the current density used in step b). In one embodiment of step a) the electrochemical oxidation is carried out at current densities from 5 to 50 A/m², wherein the current density used is stepwise decreased in steps of 1 to 10 A/m², wherein the current density used in step a) is lower than the current density used in step b).

In one embodiment of step a) the electrochemical oxidation is carried out at current densities from 10 to 5000 A/m², wherein the current density used is stepwise decreased in steps of 1 to 10 A/m², wherein the current density used in step a) is lower than the current density used in step b). In one embodiment of step a) the electrochemical oxidation is carried out at current densities from 10 to 500 A/m², wherein the current density used is stepwise decreased in steps of 1 to 10 A/m², wherein the current density used in step a) is lower than the current density used in step b). In one embodiment of step a) the electrochemical oxidation is carried out at current densities from 10 to 100 A/m², wherein the current density used is stepwise decreased in steps of 1 to 10 A/m², wherein the current density used in step a) is lower than the current density used in step b). In one embodiment of step a) the electrochemical oxidation is carried out at current densities from 10 to 50 A/m², wherein the current density used is stepwise decreased in steps of 1 to 10 A/m², wherein the current density used in step a) is lower than the current density used in step b).

In one embodiment of step a) the electrochemical oxidation is carried out at current densities from 1 to 5000 A/m², wherein the current density used is stepwise decreased in steps of 1 to 5 A/m², wherein the current density used in step a) is lower than the current density used in step b). In one embodiment of step a) the electrochemical oxidation is carried out at current densities from 1 to 500 A/m², wherein the current density used is stepwise decreased in steps of 1 to 5 A/m², wherein the current density used in step a) is lower than the current density used in step b). In one embodiment of step a) the electrochemical oxidation is carried out at current densities from 1 to 100 A/m², wherein the current density used is stepwise decreased in steps of 1 to 5 A/m², wherein the current density used in step a) is lower than the current density used in step b). In one embodiment of step a) the electrochemical oxidation is carried out at current densities from 1 to 50 A/m², wherein the current density used is stepwise decreased in steps of 1 to 5 A/m², wherein the current density used in step a) is lower than the current density used in step b).

In one embodiment of step a) the electrochemical oxidation is carried out at current densities from 5 to 5000 A/m², wherein the current density used is stepwise decreased in steps of 1 to 5 A/m², wherein the current density used in step a) is lower than the current density used in step b). In one embodiment of step a) the electrochemical oxidation is carried out at current densities from 5 to 500 A/m², wherein the current density used is stepwise decreased in steps of 1 to 5 A/m², wherein the current density used in step a) is lower than the current density used in step b). In one embodiment of step a) the electrochemical oxidation is carried out at current densities from 5 to 100 A/m², wherein the current density used is stepwise decreased in steps of 1 to 5 A/m², wherein the current density used in step a) is lower than the current density used in step b). In one embodiment of step a) the electrochemical oxidation is carried out at current densities from 5 to 50 A/m², wherein the current density used is stepwise decreased in steps of 1 to 5 A/m², wherein the current density used in step a) is lower than the current density used in step b).

In one embodiment of step a) the electrochemical oxidation is carried out at current densities from 10 to 5000 A/m², wherein the current density used is stepwise decreased in steps of 1 to 5 A/m², wherein the current density used in step a) is lower than the current density used in step b). In one embodiment of step a) the electrochemical oxidation is carried out at current densities from 10 to 500 A/m², wherein the current density used is stepwise decreased in steps of 1 to 5 A/m², wherein the current density used in step a) is lower than the current density used in step b). In one embodiment of step a) the electrochemical oxidation is carried out at current densities from 10 to 100 A/m², wherein the current density used is stepwise decreased in steps of 1 to 5 A/m², wherein the current density used in step a) is lower than the current density used in step b). In one embodiment of step a) the electrochemical oxidation is carried out at current densities from 10 to 50 A/m², wherein the current density used is stepwise decreased in steps of 1 to 5 A/m², wherein the current density used in step a) is lower than the current density used in step b).

In one embodiment of step a) the electrochemical oxidation is carried out at current densities from 1 to 5000 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the current density used in step a) is lower than the current density used in step b). In one embodiment of step a) the electrochemical oxidation is carried out at current densities from 1 to 500 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the current density used in step a) is lower than the current density used in step b). In one embodiment of step a) the electrochemical oxidation is carried out at current densities from 1 to 100 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the current density used in step a) is lower than the current density used in step b). In one embodiment of step a) the electrochemical oxidation is carried out at current densities from 1 to 50 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the current density used in step a) is lower than the current density used in step b).

In one embodiment of step a) the electrochemical oxidation is carried out at current densities from 5 to 5000 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the current density used in step a) is lower than the current density used in step b). In one embodiment of step a) the electrochemical oxidation is carried out at current densities from 5 to 500 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the current density used in step a) is lower than the current density used in step b). In one embodiment of step a) the electrochemical oxidation is carried out at current densities from 5 to 100 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m². In one embodiment of step a) the electrochemical oxidation is carried out at current densities from 5 to 50 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the current density used in step a) is lower than the current density used in step b).

In one embodiment of step a) the electrochemical oxidation is carried out at current densities from 10 to 5000 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the current density used in step a) is lower than the current density used in step b). In one embodiment of step a) the electrochemical oxidation is carried out at current densities from 10 to 500 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the current density used in step a) is lower than the current density used in step b). In one embodiment of step a) the electrochemical oxidation is carried out at current densities from 10 to 100 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the current density used in step a) is lower than the current density used in step b). In one embodiment of step a) the electrochemical oxidation is carried out at current densities from 10 to 50 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the current density used in step a) is lower than the current density used in step b).

In one embodiment of step a) the electrochemical oxidation is carried out at current densities from 1 to 5000 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are identical, wherein the current density used in step a) is lower than the current density used in step b). In one embodiment of step a) the electrochemical oxidation is carried out at current densities from 1 to 500 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are identical, wherein the current density used in step a) is lower than the current density used in step b). In one embodiment of step a) the electrochemical oxidation is carried out at current densities from 1 to 100 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are identical, wherein the current density used in step a) is lower than the current density used in step b). In one embodiment of step a) the electrochemical oxidation is carried out at current densities from 1 to 50 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are identical, wherein the current density used in step a) is lower than the current density used in step b).

In one embodiment of step a) the electrochemical oxidation is carried out at current densities from 5 to 5000 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are identical, wherein the current density used in step a) is lower than the current density used in step b). In one embodiment of step a) the electrochemical oxidation is carried out at current densities from 5 to 500 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are identical, wherein the current density used in step a) is lower than the current density used in step b). In one embodiment of step a) the electrochemical oxidation is carried out at current densities from 5 to 100 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are identical, wherein the current density used in step a) is lower than the current density used in step b). In one embodiment of step a) the electrochemical oxidation is carried out at current densities from 5 to 50 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are identical, wherein the current density used in step a) is lower than the current density used in step b).

In one embodiment of step a) the electrochemical oxidation is carried out at current densities from 10 to 5000 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are identical, wherein the current density used in step a) is lower than the current density used in step b). In one embodiment of step a) the electrochemical oxidation is carried out at current densities from 10 to 500 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are identical, wherein the current density used in step a) is lower than the current density used in step b). In one embodiment of step a) the electrochemical oxidation is carried out at current densities from 10 to 100 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are identical, wherein the current density used in step a) is lower than the current density used in step b). In one embodiment of step a) the electrochemical oxidation is carried out at current densities from 10 to 50 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are identical, wherein the current density used in step a) is lower than the current density used in step b).

In one embodiment of step a) the electrochemical oxidation is carried out at current densities from 1 to 5000 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are varying, wherein the current density used in step a) is lower than the current density used in step b). In one embodiment of step a) the electrochemical oxidation is carried out at current densities from 1 to 500 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are varying, wherein the current density used in step a) is lower than the current density used in step b). In one embodiment of step a) the electrochemical oxidation is carried out at current densities from 1 to 100 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are varying, wherein the current density used in step a) is lower than the current density used in step b). In one embodiment of step a) the electrochemical oxidation is carried out at current densities from 1 to 50 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are varying, wherein the current density used in step a) is lower than the current density used in step b).

In one embodiment of step a) the electrochemical oxidation is carried out at current densities from 5 to 5000 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are varying, wherein the current density used in step a) is lower than the current density used in step b). In one embodiment of step a) the electrochemical oxidation is carried out at current densities from 5 to 500 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are varying, wherein the current density used in step a) is lower than the current density used in step b). In one embodiment of step a) the electrochemical oxidation is carried out at current densities from 5 to 100 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are varying, wherein the current density used in step a) is lower than the current density used in step b). In one embodiment of step a) the electrochemical oxidation is carried out at current densities from 5 to 50 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are varying, wherein the current density used in step a) is lower than the current density used in step b).

In one embodiment of step a) the electrochemical oxidation is carried out at current densities from 10 to 5000 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are varying, wherein the current density used in step a) is lower than the current density used in step b). In one embodiment of step a) the electrochemical oxidation is carried out at current densities from 10 to 500 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are varying, wherein the current density used in step a) is lower than the current density used in step b). In one embodiment of step a) the electrochemical oxidation is carried out at current densities from 10 to 100 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are varying, wherein the current density used in step a) is lower than the current density used in step b). In one embodiment of step a) the electrochemical oxidation is carried out at current densities from 10 to 50 A/m², wherein the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m², wherein the steps are varying, wherein the current density used in step a) is lower than the current density used in step b).

### Step b)

In one embodiment of step b) the electrochemical reduction is carried out at a temperature from 1 to 50° C. In one embodiment of step b) the electrochemical reduction is carried out at a temperature from 10 to 35° C. In one embodiment of step b) the electrochemical reduction is carried out at a temperature from 15 to 30° C. In one embodiment of step b) the electrochemical reduction is carried out at ambient temperature.

In one embodiment of step b) a solvent is used. In one embodiment of step b) a solvent selected from aprotic solvents, protic solvent, and mixtures thereof is used. In one embodiment of step b) a solvent selected from polar aprotic solvents and mixtures thereof is used. In one embodiment of step b) a solvent selected from polar aprotic solvents selected from dimethylformamide (DMF), dimethyl sulfoxide, acetonitrile, acetone and mixtures thereof is used. In one embodiment of step b) a solvent selected from protic solvents selected from methanol, acetic acid, ethanol, formic acid, water and mixtures thereof is used. In one embodiment of step b) a solvent selected from dimethylformamide (DMF), methanol, acetic acid and mixtures thereof is used. In one embodiment of step b) a solvent selected from methanol, acetic acid and mixtures thereof is used. In one embodiment of step b) methanol is used. In one embodiment of step b) acetic acid is used.

In one embodiment of step b) a solvent selected from
aprotic solvents, protic solvent, and mixtures thereof; or
polar aprotic solvents; or
polar aprotic solvents selected from dimethylformamide (DMF), dimethyl sulfoxide, acetonitrile, acetone; or
protic solvents selected from methanol, acetic acid, ethanol, formic acid, water and mixtures thereof; or
dimethylformamide (DMF), methanol, acetic acid and mixtures thereof; or
methanol, acetic acid and mixtures thereof; or
methanol; or
acetic acid;
   or mixtures of the foregoing.

In one embodiment of step b) a conducting salt selected from the group of organic ammonia salts, ionic liquids, tetraalkylammonium fluoroborates, quaternary ammonia salts and mixtures thereof is used.

In one embodiment of step b) a conducting salt selected from the group of tetraethylammonium tetrafluoroborate, tetrabutylammonium hexafluorophosphate, tetrabutylammonium hydrogen sulfate and mixtures thereof is used.

In one embodiment of step b) the solvent is used in an amount of 80 to 99 wt.-%, based on the amount of the reaction mixture. In one embodiment of step b) the solvent is used in an amount of 85 to 95 wt.-%, based on the amount of the reaction mixture. In one embodiment of step b) the solvent is used in an amount of 85 to 92 wt.-%, based on the amount of the reaction mixture. In one embodiment of step b) the solvent is used in an amount of 90 wt.-%, based on the amount of the reaction mixture.

In one embodiment of step b)
a conducting salt selected from the group of organic ammonia salts, ionic liquids, tetraalkylammonium fluoroborates, quaternary ammonia salts and mixtures thereof; and
a solvent is selected from dimethylformamide (DMF), methanol, acetic acid and mixtures thereof
   is used.

In one embodiment of step b)
a conducting salt selected from the group of organic ammonia salts, ionic liquids, tetraalkylammonium fluoroborates, quaternary ammonia salts and mixtures thereof; and
a solvent is selected from dimethylformamide (DMF), methanol, acetic acid and mixtures thereof
   is used.

In one embodiment of step b)
a conducting salt selected from the group of tetraethylammonium tetrafluoroborate, tetrabutylammonium hexafluorophosphate, tetrabutylammonium hydrogen sulfate and mixtures thereof; and
a solvent is selected from dimethylformamide (DMF), methanol, acetic acid and mixtures thereof
   is used.

In one embodiment of step b)
a conducting salt selected from the group of organic ammonia salts, ionic liquids, tetraalkylammonium fluoroborates, quaternary ammonia salts and mixtures thereof; and
methanol
   is used.

In one embodiment of step b)
a conducting salt selected from the group of organic ammonia salts, ionic liquids tetraalkylammonium fluoroborates, quaternary ammonia salts and mixtures thereof; and
methanol
   is used.

In one embodiment of step b)
a conducting salt selected from the group of tetraethylammonium tetrafluoroborate, tetrabutylammonium hexafluorophosphate, tetrabutylammonium hydrogen sulfate and mixtures thereof; and
methanol
is used.

In one embodiment of step b)
a conducting salt selected from the group of organic ammonia salts, ionic liquids, tetraalkylammonium fluoroborates, quaternary ammonia salts and mixtures thereof; and
acetic acid
   is used.

In one embodiment of step b)
a conducting salt selected from the group of organic ammonia salts, ionic liquids tetraalkylammonium fluoroborates, quaternary ammonia salts and mixtures thereof; and
acetic acid
   is used.

In one embodiment of step b)
a conducting salt selected from the group of tetraethylammonium tetrafluoroborate, tetrabutylammonium hexafluorophosphate, tetrabutylammonium hydrogen sulfate and mixtures thereof; and
acetic acid
   is used.

In one embodiment of step b), 0.05 to 3 equivalents of conducting salt are used, based on 1 equivalent of the compound according to formula (XIII). This means, if 0.05 equivalent of conducting salt is used, 1 equivalent of the compound according to formula (XIII) is used. In one embodiment of step b), 0.2 to 2 equivalents of conducting salt are used, based on 1 equivalent of the compound according to formula (XIII). In one embodiment of step b), 0.5 to 1.5 equivalents of conducting salt are used, based on 1 equivalent of the compound according to formula (XIII). In one embodiment of step b), 0.6 to 1.3 equivalents of conducting salt are used, based on 1 equivalent of the compound according to formula ent-(I). The equivalents are based on the compound according to formula (XIII). This means if 1.3 equivalent (1.3 mol) of acid 1 equivalent (1 mol) of the compound according to formula (XIII) is used.

In one embodiment of step b) the electrochemical reduction is carried out at a cell voltage of 0.1 to 30 V. In one embodiment of step b) the electrochemical reduction is carried out at a cell voltage of 0.1 to 20 V. In one embodiment of step b) the electrochemical reduction is carried out at a cell voltage of 0.1 to 15 V. In one embodiment of step b) the electrochemical reduction is carried out at a cell voltage of 0.1 to 10 V. In one embodiment of step b) the electrochemical reduction is carried out at a cell voltage of 0.1 to 5 V.

In one embodiment of step b) the electrochemical reduction is carried out at a cell voltage of 0.5 to 30 V. In one embodiment of step b) the electrochemical reduction is carried out at a cell voltage of 0.5 to 20 V. In one embodiment of step b) the electrochemical reduction is carried out at a cell voltage of 0.5 to 15 V. In one embodiment of step b) the electrochemical reduction is carried out at a cell voltage of 0.5 to 10 V. In one embodiment of step b) the electrochemical reduction is carried out at a cell voltage of 0.5 to 5 V.

In one embodiment of step b) the electrochemical reduction is carried out at a cell voltage of 1 to 30 V. In one embodiment of step b) the electrochemical reduction is carried out at a cell voltage of 1 to 20 V. In one embodiment of step b) the electrochemical reduction is carried out at a cell voltage of 1 to 15 V. In one embodiment of step b) the electrochemical reduction is carried out at a cell voltage of 1 to 10 V. In one embodiment of step b) the electrochemical reduction is carried out at a cell voltage of 1 to 5 V.

In one embodiment of step b) the electrochemical reduction is carried out at a cell voltage of 2 to 30 V. In one embodiment of step b) the electrochemical reduction is carried out at a cell voltage of 2 to 20 V. In one embodiment of step b) the electrochemical reduction is carried out at a cell voltage of 2 to 15 V. In one embodiment of step b) the electrochemical reduction is carried out at a cell voltage of 2 to 10 V. In one embodiment of step b) the electrochemical reduction is carried out at a cell voltage of 2 to 5 V.

In one embodiment of step b) the electrochemical reduction is carried out at a cell voltage of 3 to 30 V. In one embodiment of step b) the electrochemical reduction is carried out at a cell voltage of 3 to 20 V. In one embodiment of step b) the electrochemical reduction is carried out at a cell voltage of 3 to 15 V. In one embodiment of step b) the electrochemical reduction is carried out at a cell voltage of 3 to 10 V. In one embodiment of step b) the electrochemical reduction is carried out at a cell voltage of 3 to 5 V.

In one embodiment of step b) the electrochemical reduction is carried out at a cell voltage of less than 30 V. In one embodiment of step b) the electrochemical reduction is carried out at a cell voltage of less than 20 V. In one embodiment of step b) the electrochemical reduction is carried out at a cell voltage of less than 15 V. In one embodiment of step b) the electrochemical reduction is carried out at a cell voltage of less than 10 V. In one embodiment of step b) the electrochemical reduction is carried out at a cell voltage of less than 5 V.

In one embodiment of step b) the electrochemical reduction is carried out at a cell voltage of less than 30 V, 29 V, 28 V, 27 V, 26 V, 25 V, 24 V, 23 V, 22 V, 21 V, 20 V, 19 V, 18 V, 17 V, 16 V, 15 V, 14 V, 13 V, 12V, 11V, 10V, 9V, 8V, 7V, 6V, 5V, 4V, 3V, 2V, 1V, 0.5V or 0.1V.

In one embodiment of step b) the electrochemical reduction is carried out at current densities from 1 to 10000 A/m². In one embodiment of step b) the electrochemical reduction is carried out at current densities from 1 to 1000 A/m². In one embodiment of step b) the electrochemical reduction is carried out at current densities from 1 to 500 A/m². In one embodiment of step b) the electrochemical reduction is carried out at current densities from 1 to 400 A/m². In one embodiment of step b) the electrochemical reduction is carried out at current densities from 1 to 350 A/m².

In one embodiment of step b) the electrochemical reduction is carried out at current densities from 5 to 10000 A/m². In one embodiment of step b) the electrochemical reduction is carried out at current densities from 5 to 1000 A/m². In one embodiment of step b) the electrochemical reduction is carried out at current densities from 5 to 500 A/m². In one embodiment of step b) the electrochemical reduction is carried out at current densities from 5 to 400 A/m². In one embodiment of step b) the electrochemical reduction is carried out at current densities from 5 to 350 A/m².

In one embodiment of step b) the electrochemical reduction is carried out at current densities from 10 to 10000 A/m². In one embodiment of step b) the electrochemical reduction is carried out at current densities from 10 to 1000 A/m². In one embodiment of step b) the electrochemical reduction is carried out at current densities from 10 to 500 A/m². In one embodiment of step b) the electrochemical reduction is carried out at current densities from 10 to 400 A/m². In one embodiment of step b) the electrochemical reduction is carried out at current densities from 10 to 350 A/m².

In one embodiment of step b) the electrochemical reduction is carried out at current densities from 100 to 10000 A/m². In one embodiment of step b) the electrochemical reduction is carried out at current densities from 100 to 1000 A/m². In one embodiment of step b) the electrochemical reduction is carried out at current densities from 100 to 500 A/m². In one embodiment of step b) the electrochemical reduction is carried out at current densities from 100 to 400 A/m². In one embodiment of step b) the electrochemical reduction is carried out at current densities from 100 to 350 A/m².

In one embodiment of step b) the electrochemical reduction is carried out at current densities from constant current density of 200 to 400 A/m². In one embodiment of step b) the electrochemical reduction is carried out at a constant current density of 200 A/m². In one embodiment of step b) the electrochemical reduction is carried out at a constant current density of 250 A/m². In one embodiment of step b) the electrochemical reduction is carried out at a constant current density of 300 A/m². In one embodiment of step b) the electrochemical reduction is carried out at a constant current density of 350 A/m². In one embodiment of step b) the electrochemical reduction is carried out at a constant current density of 400 A/m².

In one embodiment of step b) the electrochemical reduction is carried out at current densities from 1 To 10000 A/m², wherein the current density is constant, variable, increasing, decreasing or a specific program can also be run. In one embodiment of step b) the electrochemical reduction is carried out at current densities from 1 To 1000 A/m², wherein the current density is constant, variable, increasing, decreasing or a specific program can also be run. In one embodiment of step b) the electrochemical reduction is carried out at current densities from 1 To 500 A/m², wherein the current density is constant, variable, increasing, decreasing or a specific program can also be run. In one embodiment of step b) the electrochemical reduction is carried out at current densities from 1 To 400 A/m², wherein the current density is constant, variable, increasing, decreasing or a specific program can also be run. In one embodiment of step b) the electrochemical reduction is carried out at current densities from 1 to 350 A/m², wherein the current density is constant, variable, increasing, decreasing or a specific program can also be run.

In one embodiment of step b) the electrochemical reduction is carried out at current densities from 5 to 10000 A/m², wherein the current density is constant, variable, increasing, decreasing or a specific program can also be run. In one embodiment of step b) the electrochemical reduction is carried out at current densities from 5 to 1000 A/m², wherein the current density is constant, variable, increasing, decreasing or a specific program can also be run. In one embodiment of step b) the electrochemical reduction is carried out at current densities from 5 to 500 A/m², wherein the current density is constant, variable, increasing, decreasing or a specific program can also be run. In one embodiment of step b) the electrochemical reduction is carried out at current densities from 5 to 400 A/m², wherein the current density is constant, variable, increasing, decreasing or a specific program can also be run. In one embodiment of step b) the electrochemical reduction is carried out at current densities from 5 to 350 A/m², wherein the current density is constant, variable, increasing, decreasing or a specific program can also be run.

In one embodiment of step b) the electrochemical reduction is carried out at current densities from 10 to 10000 A/m², wherein the current density is constant, variable, increasing, decreasing or a specific program can also be run. In one embodiment of step b) the electrochemical reduction is carried out at current densities from 10 to 1000 A/m², wherein the current density is constant, variable, increasing, decreasing or a specific program can also be run. In one embodiment of step b) the electrochemical reduction is carried out at current densities from 10 to 500 A/m², wherein the current density is constant, variable, increasing, decreasing or a specific program can also be run. In one embodiment of step b) the electrochemical reduction is carried out at current densities from 10 to 400 A/m², wherein the current density is constant, variable, increasing, decreasing or a specific program can also be run. In one embodiment of step b) the electrochemical reduction is carried out at current densities from 10 to 350 A/m², wherein the current density is constant, variable, increasing, decreasing or a specific program can also be run.

In one embodiment of step b) the electrochemical reduction is carried out at current densities from 1 to 10000 A/m², wherein the current density is constant or variable. In one embodiment of step b) the electrochemical reduction is carried out at current densities from 1 to 1000 A/m², wherein the current density is constant or variable. In one embodiment of step b) the electrochemical reduction is carried out at current densities from 1 to 500 A/m², wherein the current density is constant or variable. In one embodiment of step b) the electrochemical reduction is carried out at current densities from 1 to 400 A/m², wherein the current density is constant or variable. In one embodiment of step b) the electrochemical reduction is carried out at current densities from 1 to 350 A/m², wherein the current density is constant or variable.

In one embodiment of step b) the electrochemical reduction is carried out at current densities from 5 to 10000 A/m², wherein the current density is constant or variable. In one embodiment of step b) the electrochemical reduction is carried out at current densities from 5 to 1000 A/m², wherein the current density is constant or variable. In one embodiment of step b) the electrochemical reduction is carried out at current densities from 5 to 500 A/m², wherein the current density is constant or variable. In one embodiment of step b) the electrochemical reduction is carried out at current densities from 5 to 400 A/m², wherein the current density is constant or variable. In one embodiment of step b) the electrochemical reduction is carried out at current densities from 5 to 350 A/m², wherein the current density is constant or variable.

In one embodiment of step b) the electrochemical reduction is carried out at current densities from 10 to 10000 A/m², wherein the current density is constant or variable. In one embodiment of step b) the electrochemical reduction is carried out at current densities from 10 to 1000 A/m², wherein the current density is constant or variable. In one embodiment of step b) the electrochemical reduction is carried out at current densities from 10 to 500 A/m², wherein the current density is constant or variable. In one embodiment of step b) the electrochemical reduction is carried out at current densities from 10 to 400 A/m², wherein the current density is constant or variable. In one embodiment of step b) the electrochemical reduction is carried out at current densities from 10 to 350 A/m², wherein the current density is constant or variable.

In one embodiment of step b) the electrochemical reduction is carried out at current densities from 100 to 10000 A/m², wherein the current density is constant or variable. In one embodiment of step b) the electrochemical reduction is carried out at current densities from 100 to 1000 A/m², wherein the current density is constant or variable. In one embodiment of step b) the electrochemical reduction is carried out at current densities from 100 to 500 A/m², wherein the current density is constant or variable. In one embodiment of step b) the electrochemical reduction is carried out at current densities from 100 to 400 A/m², wherein the current density is constant or variable. In one embodiment of step b) the electrochemical reduction is carried out at current densities from 100 to 350 A/m², wherein the current density is constant or variable.

In one embodiment of step b) the electrochemical reduction is carried out at current densities from constant current density of 200 to 400 A/m², wherein the current density is constant or variable. In one embodiment of step b) the electrochemical reduction is carried out at a constant current density of 200 A/m², wherein the current density is constant or variable. In one embodiment of step b) the electrochemical reduction is carried out at a constant current density of 250 A/m², wherein the current density is constant or variable. In one embodiment of step b) the electrochemical reduction is carried out at a constant current density of 300 A/m², wherein the current density is constant or variable. In one embodiment of step b) the electrochemical reduction is carried out at a constant current density of 350 A/m², wherein the current density is constant or variable. In one embodiment of step b) the electrochemical reduction is carried out at a constant current density of 400 A/m², wherein the current density is constant or variable.

In one embodiment of step b) the electrochemical reduction is carried out at current densities from 1 to 10000 A/m², wherein the current density is increased or decreased. In one embodiment of step b) the electrochemical reduction is carried out at current densities from 1 to 1000 A/m², wherein the current density is increased or decreased. In one embodiment of step b) the electrochemical reduction is carried out at current densities from 1 to 500 A/m², wherein the current density is increased or decreased. In one embodiment of step b) the electrochemical reduction is carried out at current densities from 1 to 400 A/m², wherein the current density is increased or decreased. In one embodiment of step b) the electrochemical reduction is carried out at current densities from 1 to 350 A/m², wherein the current density is increased or decreased.

In one embodiment of step b) the electrochemical reduction is carried out at current densities from 5 to 10000 A/m², wherein the current density is increased or decreased. In one embodiment of step b) the electrochemical reduction is carried out at current densities from 5 to 1000 A/m², wherein the current density is increased or decreased. In one embodiment of step b) the electrochemical reduction is carried out at current densities from 5 to 500 A/m², wherein the current density is increased or decreased. In one embodiment of step b) the electrochemical reduction is carried out at current densities from 5 to 400 A/m², wherein the current density is increased or decreased. In one embodiment of step b) the electrochemical reduction is carried out at current densities from 5 to 350 A/m², wherein the current density is increased or decreased.

In one embodiment of step b) the electrochemical reduction is carried out at current densities from 10 to 10000 A/m², wherein the current density is increased or decreased. In one embodiment of step b) the electrochemical reduction is carried out at current densities from 10 to 1000 A/m², wherein the current density is increased or decreased. In one embodiment of step b) the electrochemical reduction is carried out at current densities from 10 to 500 A/m², wherein the current density is increased or decreased. In one embodiment of step b) the electrochemical reduction is carried out at current densities from 10 to 400 A/m², wherein the current density is increased or decreased. In one embodiment of step b) the electrochemical reduction is carried out at current densities from 10 to 350 A/m², wherein the current density is increased or decreased.

In one embodiment of step b) the electrochemical reduction is carried out at current densities from 100 to 10000 A/m², wherein the current density is increased or decreased. In one embodiment of step b) the electrochemical reduction is carried out at current densities from 100 to 1000 A/m², wherein the current density is increased or decreased. In one embodiment of step b) the electrochemical reduction is carried out at current densities from 100 to 500 A/m², wherein the current density is increased or decreased. In one embodiment of step b) the electrochemical reduction is carried out at current densities from 100 to 400 A/m², wherein the current density is increased or decreased. In one embodiment of step b) the electrochemical reduction is carried out at current densities from 100 t 350 A/m², wherein the current density is increased or decreased.

In one embodiment of step b) the electrochemical reduction is carried out at current densities from constant current density of 200 to 400 A/m², wherein the current density is increased or decreased. In one embodiment of step b) the electrochemical reduction is carried out at a constant current density of 200 A/m², wherein the current density is increased or decreased. In one embodiment of step b) the electrochemical reduction is carried out at a constant current density of 250 A/m², wherein the current density is increased or decreased. In one embodiment of step b) the electrochemical reduction is carried out at a constant current density of 300 A/m², wherein the current density is increased or decreased. In one embodiment of step b) the electrochemical reduction is carried out at a constant current density of 350 A/m², wherein the current density is increased or decreased. In one embodiment of step b) the electrochemical reduction is carried out at a constant current density of 400 A/m², wherein the current density is increased or decreased.

### Step c)

In one embodiment, the sequential one-pot synthesis further comprises step c)
Step c) isolation of the compound according to formula (XIII).

In one embodiment of step c) the isolation is selected from filtration and crystallization. In one embodiment of step c) the compound according to formula (XIII) is isolated via filtration. In one embodiment of step c) the compound according to formula (XIII) is isolated via crystallization.

### Step d)

In one embodiment, the sequential one-pot synthesis further comprises step d)

Step d) Crystallization of the compound according to formula (XIII).

In one embodiment of step d), crystallization is carried out in an organic solvent or mixtures of organic solvents. In one embodiment of step c) the isolation is selected from chiral chromatography, crystallization, racemic resolution, diastereomeric salt formation and chiral salt formation. For example, isolation via diastereomeric salt formation is described in US20100136142 A1, US20170217957 A1 or WO2019206909 A1.

In one embodiment of step d), crystallization is carried out in organic solvents. In one embodiment of step d), crystallization is carried out in isopropanol, n-propanol or mixtures thereof. In one embodiment, compound (XIII) is first dissolved and then cooled. In one embodiment, compound (XIII) is first dissolved by heating to above 90° C. and then cooled to 10° C. In one embodiment, compound (XIII) is first dissolved by heating to above 85° C. and then cooled to 5° C. In one embodiment, compound (XIII) is first dissolved by heating to above 75° C. and then cooled to 0° C. These embodiments can lead to a very pure form of the compound according to formula (XIII).

### FURTHER EMBODIMENTS

In one embodiment, the current density used in step a) is lower than the current density used in step b). The oxidation selectivity was surprisingly so high that no secondary components were raised up. Such components can interfere with the racemization and reduction step and thus no isolation and purge after each step would be required.

In one embodiment,
the electrochemical oxidation in step a) is carried out at current densities selected from 1 to 5000 A/m², 1 to 500 A/m², 1 to 100 A/m², 1 to 50 A/m², 5 to 5000 A/m², 5 to 500 A/m², 5 to 100 A/m², 5 to 50 A/m², 10 to 5000 A/m², 10 to 500 A/m², 10 to 100 A/m², and 10 to 50 A/m²; and
the electrochemical reduction in step b) is carried out at current densities selected from 1 to 10000 A/m², 1 A/m² to 1000 A/m², 1 A/m² to 500 A/m², 5 A/m² to 10000 A/m², 50 to 1000 A/m², 200 to 500 A/m², 10 to 10000 A/m², 10 to 1000 A/m², 10 to 500 A/m², 100 to 500 A/m², 200 to 400 A/m², 200 A/m², 250 A/m², 300 A/m², 350 A/m² and 400 A/m².

In one embodiment,
the electrochemical oxidation in step a) is carried out at current densities from 1 to 5000 A/m²; and
the electrochemical reduction in step b) is carried out at current densities from 1 to 10000 A/m².

In one embodiment,
the electrochemical oxidation in step a) is carried out at current densities from 1 to 500 A/m²; and
the electrochemical reduction in step b) is carried out at current densities from 1 to 1000 A/m².

In one embodiment,
the electrochemical oxidation in step a) is carried out at current densities from 1 to 500 A/m²; and
the electrochemical reduction in step b) is carried out at current densities selected from 1 to 500 A/m².

In one embodiment,
the electrochemical oxidation in step a) is carried out at current densities from 1 to 100 A/m²; and
the electrochemical reduction in step b) is carried out at current densities from 1 A/m² to 500 A/m².

In one embodiment,
the electrochemical oxidation in step a) is carried out at current densities from 1 to 100 A/m²; and
the electrochemical reduction in step b) is carried out at current densities from 10 A/m² to 500 A/m².

In one embodiment,
the electrochemical oxidation in step a) is carried out at current densities from 1 to 50 A/m²; and
the electrochemical reduction in step b) is carried out at current densities from 10 A/m² to 500 A/m².

In one embodiment,
the electrochemical oxidation in step a) is carried out at current densities selected 1 to 100 A/m²; and
the electrochemical reduction in step b) is carried out at current densities selected 100 to 500 A/m².

In one embodiment,
the electrochemical oxidation in step a) is carried out at current densities selected 1 to 50 A/m²; and
the electrochemical reduction in step b) is carried out at current densities selected 100 to 500 A/m².

In one embodiment,
the electrochemical oxidation in step a) is carried out at current densities selected 1 to 100 A/m²; and
the electrochemical reduction in step b) is carried out at current densities selected 200 to 400 A/m².

In one embodiment,
the electrochemical oxidation in step a) is carried out at current densities selected 1 to 50 A/m²; and
the electrochemical reduction in step b) is carried out at current densities selected 200 to 400 A/m².

In one embodiment,
the electrochemical oxidation in step a) is carried out at current densities selected from 1 to 5000 A/m², 1 to 500 A/m², 1 to 100 A/m², 1 to 50 A/m², 5 to 5000 A/m², 5 to 500 A/m², 5 to 100 A/m², 5 to 50 A/m², 10 to 5000 A/m², 10 to 500 A/m², 10 to 100 A/m², and 10 to 50 A/m², wherein in step a) the current density used is decreased; and
the electrochemical reduction in step b) is carried out at current densities selected from 1 to 10000 A/m², 1 to 1000 A/m², 1 to 500 A/m², 5 to 10000 A/m², 50 to 1000 A/m², 200 to 500 A/m², 10 to 10000 A/m², 10 to 1000 A/m², 10 to 500 A/m², 100 to 500 A/m², 200 to 400 A/m², 200 A/m², 250 A/m², 300 A/m², 350 A/m² and 400 A/m².

In one embodiment,
the electrochemical oxidation in step a) is carried out at current densities from 1 to 5000 A/m², wherein in step a) the current density used is decreased; and
the electrochemical reduction in step b) is carried out at current densities from 1 to 10000 A/m².

In one embodiment,
the electrochemical oxidation in step a) is carried out at current densities from 1 to 500 A/m², wherein in step a) the current density used is decreased; and
the electrochemical reduction in step b) is carried out at current densities from 1 to 1000 A/m².

In one embodiment,
the electrochemical oxidation in step a) is carried out at current densities from 1 to 500 A/m², wherein in step a) the current density used is decreased; and
the electrochemical reduction in step b) is carried out at current densities selected from 1 to 500 A/m².

In one embodiment,
the electrochemical oxidation in step a) is carried out at current densities from 1 to 100 A/m², wherein in step a) the current density used is decreased; and
the electrochemical reduction in step b) is carried out at current densities from 1 A/m² to 500 A/m².

In one embodiment,
the electrochemical oxidation in step a) is carried out at current densities from 1 to 100 A/m², wherein in step a) the current density used is decreased; and
the electrochemical reduction in step b) is carried out at current densities from 10 A/m² to 500 A/m².

In one embodiment,
the electrochemical oxidation in step a) is carried out at current densities from 1 to 50 A/m², wherein in step a) the current density used is decreased; and
the electrochemical reduction in step b) is carried out at current densities from 10 A/m² to 500 A/m².

In one embodiment,
the electrochemical oxidation in step a) is carried out at current densities selected 1 to 100 A/m², wherein in step a) the current density used is decreased; and
the electrochemical reduction in step b) is carried out at current densities selected 100 to 500 A/m².

In one embodiment,
the electrochemical oxidation in step a) is carried out at current densities selected 1 to 50 A/m², wherein in step a) the current density used is decreased; and
the electrochemical reduction in step b) is carried out at current densities selected 100 to 500 A/m².

In one embodiment,
the electrochemical oxidation in step a) is carried out at current densities selected 1 to 100 A/m², wherein in step a) the current density used is decreased; and
the electrochemical reduction in step b) is carried out at current densities selected 200 to 400 A/m².

In one embodiment,
the electrochemical oxidation in step a) is carried out at current densities selected 1 to 50 A/m², wherein in step a) the current density used is decreased; and
the electrochemical reduction in step b) is carried out at current densities selected 200 to 400 A/m².

In one embodiment,
the electrochemical oxidation in step a) is carried out at current densities selected from 1 to 5000 A/m², 1 to 500 A/m², 1 to 100 A/m², 1 to 50 A/m², 5 to 5000 A/m², 5 to 500 A/m², 5 to 100 A/m², 5 to 50 A/m², 10 to 5000 A/m², 10 to 500 A/m², 10 to 100 A/m², and 10 to 50 A/m², wherein in step a) the current density used is stepwise decreased; and
the electrochemical reduction in step b) is carried out at current densities selected from 1 to 10000 A/m², 1 A/m² to 1000 A/m², 1 A/m² to 500 A/m², 5 A/m² to 10000 A/m², 50 to 1000 A/m², 200 to 500 A/m², 10 to 10000 A/m², 10 to 1000 A/m², 10 to 500 A/m², 100 to 500 A/m², 200 to 400 A/m², 200 A/m², 250 A/m², 300 A/m², 350 A/m² and 400 A/m².

In one embodiment,
the electrochemical oxidation in step a) is carried out at current densities from 1 to 5000 A/m², wherein in step a) the current density used is stepwise decreased; and
the electrochemical reduction in step b) is carried out at current densities from 1 to 10000 A/m².

In one embodiment,
the electrochemical oxidation in step a) is carried out at current densities from 1 to 500 A/m², wherein in step a) the current density used is stepwise decreased; and
the electrochemical reduction in step b) is carried out at current densities from 1 to 1000 A/m².

In one embodiment,
the electrochemical oxidation in step a) is carried out at current densities from 1 to 500 A/m², wherein in step a) the current density used is stepwise decreased; and
the electrochemical reduction in step b) is carried out at current densities selected from 1 to 500 A/m².

In one embodiment,
the electrochemical oxidation in step a) is carried out at current densities from 1 to 100 A/m², wherein in step a) the current density used is stepwise decreased; and
the electrochemical reduction in step b) is carried out at current densities from 1 A/m² to 500 A/m².

In one embodiment,
the electrochemical oxidation in step a) is carried out at current densities from 1 to 100 A/m², wherein in step a) the current density used is stepwise decreased; and
the electrochemical reduction in step b) is carried out at current densities from 10 A/m² to 500 A/m².

In one embodiment,
the electrochemical oxidation in step a) is carried out at current densities from 1 to 50 A/m², wherein in step a) the current density used is stepwise decreased; and
the electrochemical reduction in step b) is carried out at current densities from 10 A/m² to 500 A/m².

In one embodiment,
the electrochemical oxidation in step a) is carried out at current densities selected 1 to 100 A/m², wherein in step a) the current density used is stepwise decreased; and
the electrochemical reduction in step b) is carried out at current densities selected 100 to 500 A/m².

In one embodiment,
the electrochemical oxidation in step a) is carried out at current densities selected 1 to 50 A/m², wherein in step a) the current density used is stepwise decreased; and
the electrochemical reduction in step b) is carried out at current densities selected 100 to 500 A/m².

In one embodiment,
the electrochemical oxidation in step a) is carried out at current densities selected 1 to 100 A/m², wherein in step a) the current density used is stepwise decreased; and
the electrochemical reduction in step b) is carried out at current densities selected 200 to 400 A/m².

In one embodiment,
the electrochemical oxidation in step a) is carried out at current densities selected 1 to 50 A/m², wherein in step a) the current density used is stepwise decreased; and
the electrochemical reduction in step b) is carried out at current densities selected 200 to 400 A/m².

In one embodiment,
the electrochemical oxidation in step a) is carried out at current densities selected from 1 to 5000 A/m², 1 to 500 A/m², 1 to 100 A/m², 1 to 50 A/m², 5 to 5000 A/m², 5 to 500 A/m², 5 to 100 A/m², 5 to 50 A/m², 10 to 5000 A/m², 10 to 500 A/m², 10 to 100 A/m², and 10 to 50 A/m², wherein in step a) the current density used is decreased; and
the electrochemical reduction in step b) is carried out at current densities selected from 1 to 10000 A/m², 1 to 1000 A/m², 1 to 500 A/m², 5 A/m² to 10000 A/m², 50 to 1000 A/m², 200 to 500 A/m², 10 to 10000 A/m², 10 to 1000 A/m², 10 to 500 A/m², 100 to 500 A/m², 200 to 400 A/m², 200 A/m², 250 A/m², 300 A/m², 350 A/m² and 400 A/m²; the current density used in step a) is lower than the current density used in step b).

In one embodiment,
the electrochemical oxidation in step a) is carried out at current densities from 1 to 5000 A/m², wherein in step a) the current density used is decreased; and
the electrochemical reduction in step b) is carried out at current densities from 1 to 10000 A/m²;
the current density used in step a) is lower than the current density used in step b).

In one embodiment,
the electrochemical oxidation in step a) is carried out at current densities from 1 to 500 A/m², wherein in step a) the current density used is decreased; and
the electrochemical reduction in step b) is carried out at current densities from 1 to 1000 A/m²; the current density used in step a) is lower than the current density used in step b).

In one embodiment,
the electrochemical oxidation in step a) is carried out at current densities from 1 to 500 A/m², wherein in step a) the current density used is decreased; and
the electrochemical reduction in step b) is carried out at current densities selected from 1 to 500 A/m²; the current density used in step a) is lower than the current density used in step b).

In one embodiment,
the electrochemical oxidation in step a) is carried out at current densities from 1 to 100 A/m², wherein in step a) the current density used is decreased; and
the electrochemical reduction in step b) is carried out at current densities from 1 A/m² to 500 A/m²; the current density used in step a) is lower than the current density used in step b).

In one embodiment,
the electrochemical oxidation in step a) is carried out at current densities from 1 to 100 A/m², wherein in step a) the current density used is decreased; and
the electrochemical reduction in step b) is carried out at current densities from 10 A/m² to 500 A/m²; the current density used in step a) is lower than the current density used in step b).

In one embodiment,
the electrochemical oxidation in step a) is carried out at current densities from 1 to 50 A/m², wherein in step a) the current density used is decreased; and
the electrochemical reduction in step b) is carried out at current densities from 10 A/m² to 500 A/m²; the current density used in step a) is lower than the current density used in step b).

In one embodiment,
the electrochemical oxidation in step a) is carried out at current densities selected 1 to 100 A/m², wherein in step a) the current density used is decreased; and
the electrochemical reduction in step b) is carried out at current densities selected 100 to 500 A/m²; the current density used in step a) is lower than the current density used in step b).

In one embodiment,
the electrochemical oxidation in step a) is carried out at current densities selected 1 to 50 A/m², wherein in step a) the current density used is decreased; and
the electrochemical reduction in step b) is carried out at current densities selected 100 to 500 A/m²; the current density used in step a) is lower than the current density used in step b).

In one embodiment,
the electrochemical oxidation in step a) is carried out at current densities selected 1 to 100 A/m², wherein in step a) the current density used is decreased; and
the electrochemical reduction in step b) is carried out at current densities selected 200 to 400 A/m²; the current density used in step a) is lower than the current density used in step b).

In one embodiment,
the electrochemical oxidation in step a) is carried out at current densities selected 1 to 50 A/m², wherein in step a) the current density used is decreased; and
the electrochemical reduction in step b) is carried out at current densities selected 200 to 400 A/m²; the current density used in step a) is lower than the current density used in step b).

In one embodiment,
the electrochemical oxidation in step a) is carried out at current densities selected from 1 to 5000 A/m², 1 to 500 A/m², 1 to 100 A/m², 1 to 50 A/m², 5 to 5000 A/m², 5 to 500 A/m², 5 to 100 A/m², 5 to 50 A/m², 10 to 5000 A/m², 10 to 500 A/m², 10 to 100 A/m², and 10 to 50 A/m², wherein in step a) the current density used is stepwise decreased; and
the electrochemical reduction in step b) is carried out at current densities selected from 1 to 10000 A/m², 1 to 1000 A/m², 1 to 500 A/m², 5 A/m² to 10000 A/m², 50 to 1000 A/m², 200 to 500 A/m², 10 to 10000 A/m², 10 to 1000 A/m², 10 to 500 A/m², 100 to 500 A/m², 200 to 400 A/m², 200 A/m², 250 A/m², 300 A/m², 350 A/m² and 400 A/m²; the current density used in step a) is lower than the current density used in step b).

In one embodiment,
the electrochemical oxidation in step a) is carried out at current densities from 1 to 5000 A/m², wherein in step a) the current density used is stepwise decreased; and
the electrochemical reduction in step b) is carried out at current densities from 1 to 10000 A/m²; the current density used in step a) is lower than the current density used in step b).

In one embodiment,
the electrochemical oxidation in step a) is carried out at current densities from 1 to 500 A/m², wherein in step a) the current density used is stepwise decreased; and
the electrochemical reduction in step b) is carried out at current densities from 1 to 1000 A/m²; the current density used in step a) is lower than the current density used in step b).

In one embodiment,
the electrochemical oxidation in step a) is carried out at current densities from 1 to 500 A/m², wherein in step a) the current density used is stepwise decreased; and
the electrochemical reduction in step b) is carried out at current densities selected from 1 to 500 A/m²; the current density used in step a) is lower than the current density used in step b).

In one embodiment,
the electrochemical oxidation in step a) is carried out at current densities from 1 to 100 A/m², wherein in step a) the current density used is stepwise decreased; and
the electrochemical reduction in step b) is carried out at current densities from 1 A/m² to 500 A/m²; the current density used in step a) is lower than the current density used in step b).

In one embodiment,
the electrochemical oxidation in step a) is carried out at current densities from 1 to 100 A/m², wherein in step a) the current density used is stepwise decreased; and
the electrochemical reduction in step b) is carried out at current densities from 10 A/m² to 500 A/m²; the current density used in step a) is lower than the current density used in step b).

In one embodiment,
the electrochemical oxidation in step a) is carried out at current densities from 1 to 50 A/m², wherein in step a) the current density used is stepwise decreased; and
the electrochemical reduction in step b) is carried out at current densities from 10 A/m² to 500 A/m²; the current density used in step a) is lower than the current density used in step b).

In one embodiment,
the electrochemical oxidation in step a) is carried out at current densities selected 1 to 100 A/m², wherein in step a) the current density used is stepwise decreased; and
the electrochemical reduction in step b) is carried out at current densities selected 100 to 500 A/m²; the current density used in step a) is lower than the current density used in step b).

In one embodiment,
the electrochemical oxidation in step a) is carried out at current densities selected 1 to 50 A/m², wherein in step a) the current density used is stepwise decreased; and
the electrochemical reduction in step b) is carried out at current densities selected 100 to 500 A/m²; the current density used in step a) is lower than the current density used in step b).

In one embodiment,
the electrochemical oxidation in step a) is carried out at current densities selected 1 to 100 A/m², wherein in step a) the current density used is stepwise decreased; and
the electrochemical reduction in step b) is carried out at current densities selected 200 to 400 A/m²; the current density used in step a) is lower than the current density used in step b).

In one embodiment,
the electrochemical oxidation in step a) is carried out at current densities selected 1 to 50 A/m², wherein in step a) the current density used is stepwise decreased; and
the electrochemical reduction in step b) is carried out at current densities selected 200 to 400 A/m²; the current density used in step a) is lower than the current density used in step b).

In one embodiment,
the electrochemical oxidation in step a) is carried out at current densities selected from 1 to 5000 A/m², 1 to 500 A/m², 1 to 100 A/m², 1 to 50 A/m², 5 to 5000 A/m², 5 to 500 A/m², 5 to 100 A/m², 5 to 50 A/m², 10 to 5000 A/m², 10 to 500 A/m², 10 to 100 A/m², and 10 to 50 A/m², wherein in step a) the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m²; and
the electrochemical reduction in step b) is carried out at current densities selected from 1 to 10000 A/m², 1 A/m² to 1000 A/m², 1 to 500 A/m², 5 to 10000 A/m², 50 to 1000 A/m², 200 to 500 A/m², 10 to 10000 A/m², 10 to 1000 A/m², 10 to 500 A/m², 100 to 500 A/m², 200 to 400 A/m², 200 A/m², 250 A/m², 300 A/m², 350 A/m² and 400 A/m²; the current density used in step a) is lower than the current density used in step b).

In one embodiment,
the electrochemical oxidation in step a) is carried out at current densities from 1 to 5000 A/m², wherein in step a) the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m²; and
the electrochemical reduction in step b) is carried out at current densities from 1 to 10000 A/m²; the current density used in step a) is lower than the current density used in step b).

In one embodiment,
the electrochemical oxidation in step a) is carried out at current densities from 1 to 500 A/m², wherein in step a) the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m²; and
the electrochemical reduction in step b) is carried out at current densities from 1 to 1000 A/m²; the current density used in step a) is lower than the current density used in step b).

In one embodiment,
the electrochemical oxidation in step a) is carried out at current densities from 1 to 500 A/m², wherein in step a) the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m²; and
the electrochemical reduction in step b) is carried out at current densities selected from 1 to 500 A/m²; the current density used in step a) is lower than the current density used in step b).

In one embodiment,
the electrochemical oxidation in step a) is carried out at current densities from 1 to 100 A/m², wherein in step a) the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m²; and
the electrochemical reduction in step b) is carried out at current densities from 1 A/m² to 500 A/m²; the current density used in step a) is lower than the current density used in step b).

In one embodiment,
the electrochemical oxidation in step a) is carried out at current densities from 1 to 100 A/m², wherein in step a) the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m²; and
the electrochemical reduction in step b) is carried out at current densities from 10 A/m² to 500 A/m²; the current density used in step a) is lower than the current density used in step b).

In one embodiment,
the electrochemical oxidation in step a) is carried out at current densities from 1 to 50 A/m², wherein in step a) the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m²; and
the electrochemical reduction in step b) is carried out at current densities from 10 A/m² to 500 A/m²; the current density used in step a) is lower than the current density used in step b).

In one embodiment,
the electrochemical oxidation in step a) is carried out at current densities selected 1 to 100 A/m², wherein in step a) the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m²; and
the electrochemical reduction in step b) is carried out at current densities selected 100 to 500 A/m²; the current density used in step a) is lower than the current density used in step b).

In one embodiment,
the electrochemical oxidation in step a) is carried out at current densities selected 1 to 50 A/m², wherein in step a) the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m²; and
the electrochemical reduction in step b) is carried out at current densities selected 100 to 500 A/m²; the current density used in step a) is lower than the current density used in step b).

In one embodiment,
the electrochemical oxidation in step a) is carried out at current densities selected 1 to 100 A/m², wherein in step a) the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50; and
the electrochemical reduction in step b) is carried out at current densities selected 200 to 400 A/m²; the current density used in step a) is lower than the current density used in step b).

In one embodiment,
the electrochemical oxidation in step a) is carried out at current densities selected 1 to 50 A/m², wherein in step a) the current density used is stepwise decreased in steps selected from 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 A/m²; and
the electrochemical reduction in step b) is carried out at current densities selected 200 to 400 A/m²; the current density used in step a) is lower than the current density used in step b).

In one embodiment,
the electrochemical oxidation in step a) is carried out at current densities selected from 1 to 5000 A/m², 1 to 500 A/m², 1 to 100 A/m², 1 to 50 A/m², 5 to 5000 A/m², 5 to 500 A/m², 5 to 100 A/m², 5 to 50 A/m², 10 to 5000 A/m², 10 to 500 A/m², 10 to 100 A/m², and 10 to 50 A/m², wherein in step a) the current density used is stepwise decreased in steps of 1 to 100 A/m²; and
the electrochemical reduction in step b) is carried out at current densities selected from 1 to 10000 A/m², 1 to 1000 A/m², 1 to 500 A/m², 5 A/m² to 10000 A/m², 50 to 1000 A/m², 200 to 500 A/m², 10 to 10000 A/m², 10 to 1000 A/m², 10 to 500 A/m², 100 to 500 A/m², 200 to 400 A/m², 200 A/m², 250 A/m², 300 A/m², 350 A/m² and 400 A/m²; the current density used in step a) is lower than the current density used in step b).

In one embodiment,
the electrochemical oxidation in step a) is carried out at current densities from 1 to 5000 A/m², wherein in step a) the current density used is stepwise decreased in steps of 1 to 100 A/m²; and
the electrochemical reduction in step b) is carried out at current densities from 1 to 10000 A/m²; the current density used in step a) is lower than the current density used in step b).

In one embodiment,
the electrochemical oxidation in step a) is carried out at current densities from 1 to 500 A/m², wherein in step a) the current density used is stepwise decreased in steps of 1 to 100 A/m²; and
the electrochemical reduction in step b) is carried out at current densities from 1 to 1000 A/m²; the current density used in step a) is lower than the current density used in step b).

In one embodiment,
the electrochemical oxidation in step a) is carried out at current densities from 1 to 500 A/m², wherein in step a) the current density used is stepwise decreased in steps of 1 to 100 A/m²; and
the electrochemical reduction in step b) is carried out at current densities selected from 1 to 500 A/m²; the current density used in step a) is lower than the current density used in step b).

In one embodiment,
the electrochemical oxidation in step a) is carried out at current densities from 1 to 100 A/m², wherein in step a) the current density used is stepwise decreased in steps of 1 to 100 A/m²; and
the electrochemical reduction in step b) is carried out at current densities from 1 A/m² to 500 A/m²; the current density used in step a) is lower than the current density used in step b).

In one embodiment,
the electrochemical oxidation in step a) is carried out at current densities from 1 to 100 A/m², wherein in step a) the current density used is stepwise decreased in steps of 1 to 100 A/m²; and
the electrochemical reduction in step b) is carried out at current densities from 10 A/m² to 500 A/m²; the current density used in step a) is lower than the current density used in step b).

In one embodiment,
the electrochemical oxidation in step a) is carried out at current densities from 1 to 50 A/m², wherein in step a) the current density used is stepwise decreased in steps of 1 to 100 A/m²; and
the electrochemical reduction in step b) is carried out at current densities from 10 A/m² to 500 A/m²; the current density used in step a) is lower than the current density used in step b).

In one embodiment,
the electrochemical oxidation in step a) is carried out at current densities selected 1 to 100 A/m², wherein in step a) the current density used is stepwise decreased in steps of 1 to 100 A/m²; and
the electrochemical reduction in step b) is carried out at current densities selected 100 to 500 A/m²; the current density used in step a) is lower than the current density used in step b).

In one embodiment,
the electrochemical oxidation in step a) is carried out at current densities selected 1 to 50 A/m², wherein in step a) the current density used is stepwise decreased in steps of 1 to 100 A/m²; and
the electrochemical reduction in step b) is carried out at current densities selected 100 to 500 A/m²; the current density used in step a) is lower than the current density used in step b).

In one embodiment,
the electrochemical oxidation in step a) is carried out at current densities selected 1 to 100 A/m², wherein in step a) the current density used is stepwise decreased in steps of 1 to 100 A/m²; and
the electrochemical reduction in step b) is carried out at current densities selected 200 to 400 A/m²; the current density used in step a) is lower than the current density used in step b).

In one embodiment,
the electrochemical oxidation in step a) is carried out at current densities selected 1 to 50 A/m², wherein in step a) the current density used is stepwise decreased in steps of 1 to 100 A/m²; and
the electrochemical reduction in step b) is carried out at current densities selected 200 to 400 A/m²; the current density used in step a) is lower than the current density used in step b).

In one embodiment,
the electrochemical oxidation in step a) is carried out at current densities selected from 1 to 5000 A/m², 1 to 500 A/m², 1 to 100 A/m², 1 to 50 A/m², 5 to 5000 A/m², 5 to 500 A/m², 5 to 100 A/m², 5 to 50 A/m², 10 to 5000 A/m², 10 to 500 A/m², 10 to 100 A/m², and 10 to 50 A/m², wherein in step a) the current density used is stepwise decreased in steps of 1 to 10 A/m²; and
the electrochemical reduction in step b) is carried out at current densities selected from 1 to 10000 A/m², 1 to 1000 A/m², 1 to 500 A/m², 5 A/m² to 10000 A/m², 50 to 1000 A/m², 200 to 500 A/m², 10 to 10000 A/m², 10 to 1000 A/m², 10 to 500 A/m², 100 to 500 A/m², 200 to 400 A/m², 200 A/m², 250 A/m², 300 A/m², 350 A/m² and 400 A/m²; the current density used in step a) is lower than the current density used in step b).

In one embodiment,
the electrochemical oxidation in step a) is carried out at current densities from 1 to 5000 A/m², wherein in step a) the current density used is stepwise decreased in steps of 1 to 10 A/m²; and
the electrochemical reduction in step b) is carried out at current densities from 1 to 10000 A/m²; the current density used in step a) is lower than the current density used in step b).

In one embodiment,
the electrochemical oxidation in step a) is carried out at current densities from 1 to 500 A/m², wherein in step a) the current density used is stepwise decreased in steps of 1 to 10 A/m²; and
the electrochemical reduction in step b) is carried out at current densities from 1 to 1000 A/m²; the current density used in step a) is lower than the current density used in step b).

In one embodiment,
the electrochemical oxidation in step a) is carried out at current densities from 1 to 500 A/m², wherein in step a) the current density used is stepwise decreased in steps of 1 to 10 A/m²; and
the electrochemical reduction in step b) is carried out at current densities selected from 1 to 500 A/m²; the current density used in step a) is lower than the current density used in step b).

In one embodiment,
the electrochemical oxidation in step a) is carried out at current densities from 1 to 100 A/m², wherein in step a) the current density used is stepwise decreased in steps of 1 to 10 A/m²; and
the electrochemical reduction in step b) is carried out at current densities from 1 A/m² to 500 A/m²; the current density used in step a) is lower than the current density used in step b).

In one embodiment,
the electrochemical oxidation in step a) is carried out at current densities from 1 to 100 A/m², wherein in step a) the current density used is stepwise decreased in steps of 1 to 10 A/m²; and
the electrochemical reduction in step b) is carried out at current densities from 10 A/m² to 500 A/m²; the current density used in step a) is lower than the current density used in step b).

In one embodiment,
the electrochemical oxidation in step a) is carried out at current densities from 1 to 50 A/m², wherein in step a) the current density used is stepwise decreased in steps of 1 to 10 A/m²; and
the electrochemical reduction in step b) is carried out at current densities from 10 A/m² to 500 A/m²; the current density used in step a) is lower than the current density used in step b).

In one embodiment,
the electrochemical oxidation in step a) is carried out at current densities selected 1 to 100 A/m², wherein in step a) the current density used is stepwise decreased in steps of 1 to 10 A/m²; and
the electrochemical reduction in step b) is carried out at current densities selected 100 to 500 A/m²; the current density used in step a) is lower than the current density used in step b).

In one embodiment,
the electrochemical oxidation in step a) is carried out at current densities selected 1 to 50 A/m², wherein in step a) the current density used is stepwise decreased in steps of 1 to 10 A/m²; and
the electrochemical reduction in step b) is carried out at current densities selected 100 to 500 A/m²; the current density used in step a) is lower than the current density used in step b).

In one embodiment,
the electrochemical oxidation in step a) is carried out at current densities selected 1 to 100 A/m², wherein in step a) the current density used is stepwise decreased in steps of 1 to 10 A/m²; and
the electrochemical reduction in step b) is carried out at current densities selected 200 to 400 A/m²; the current density used in step a) is lower than the current density used in step b).

In one embodiment,
the electrochemical oxidation in step a) is carried out at current densities selected 1 to 50 A/m², wherein in step a) the current density used is stepwise decreased in steps of 1 to 10 A/m²; and
the electrochemical reduction in step b) is carried out at current densities selected 200 to 400 A/m²; the current density used in step a) is lower than the current density used in step b).

In one embodiment, the current density used in step a) is decreased or stepwise decreased.

In one embodiment, current density used in step a) is lower than the current density used in step b).

In one embodiment, step a) is conducted at temperatures selected from 15 to 150° C., 15 to 120° C., and 20 to 100° C.

In one embodiment, in step a) first the electrochemical oxidation is conducted and then the reaction mixture is heated; or wherein first the electrochemical oxidation is conducted and then the reaction mixture is heated to temperature selected from 50 to 150° C., 75 to 150° C., to 80 to 150° C., 80 to 140° C. and 90 to 120° C.

In one embodiment, in step a) and/or b) a conducting salt is used and the conducting salt selected from the group of organic ammonia salts, ionic liquids, tetraalkylammonium fluoroborates, quaternary ammonia salts and mixtures thereof is used; or selected from the group of tetraethylammonium tetrafluoroborate, tetrabutylammonium hexafluorophosphate, tetrabutylammonium hydrogen sulfate and mixtures thereof is used.

In one embodiment, in step a) and/or b) the solvent is selected from
aprotic solvents, protic solvent, and mixtures thereof or is selected from polar aprotic solvents; or
polar aprotic solvents selected from dimethylformamide (DMF), dimethyl sulfoxide, acetonitrile, acetone; or
protic solvents selected from methanol, acetic acid, ethanol, formic acid, water and mixtures thereof; or
dimethylformamide (DMF), methanol, acetic acid and mixtures thereof; or
methanol, acetic acid and mixtures thereof; or
methanol; or
acetic acid;
or mixtures of the foregoing.

In one embodiment, in step a) an acid is used or wherein the acid used is selected from
organic acids, sulfonic acids, organic polar acids and mixtures thereof;
acetic acid, formic acid and mixtures thereof is used;
acetic acid;
and mixtures thereof.

In one embodiment, in step a) the electrochemical oxidation is carried out at a cell voltage from 0.1 to 20 V or is less than 20 V.

In one embodiment, in step a) the electrochemical oxidation is carried out at current densities selected from 1 to 5000 A/m², 1 to 500 A/m², 1 to 100 A/m², 1 to 50 A/m², 5 to 5000 A/m², 5 to 500 A/m², 5 to 100 A/m², 5 to 50 A/m², 10 to 5000 A/m², 10 to 500 A/m², 10 to 100 A/m², and 10 to 50 A/m².

In one embodiment, in step b) the electrochemical reduction is carried out at a cell voltage from 0.1 to 30 V or is less than 30 V.

In one embodiment, in step b) the electrochemical reduction is carried out at current densities selected from 1 to 10000 A/m², 1 to 1000 A/m², 1 to 500 A/m², 5 to 10000 A/m², 50 to 1000 A/m², 200 to 500 A/m², 10 to 10000 A/m², 10 to 1000 A/m², 100 to 500 A/m², 200 to 400 A/m², 200 A/m², 250 A/m², 300 A/m², 350 A/m² and 400 A/m².

In one embodiment, in step b) the electrochemical reduction is carried out at a temperature from 1 to 50° C., 10 to 35° C., 15 to 30° C., or at ambient temperature.

In one embodiment, the synthesis further comprises step c)

Step c) isolation of the compound according to formula (XIII).
wherein in step c), the isolation is optionally selected from filtration and crystallization,
and/or step d)

Step d) Crystallization of the compound according to formula (XIII).

In one embodiment,
the current density used in step a) is decreased or stepwise decreased;
current density used in step a) is lower than the current density used in step b);
step a) is conducted at temperatures selected from 20 to 120° C.,
in step a) and/or b) a conducting salt is used and the conducting salt selected from the group of organic ammonia salts, ionic liquids, tetraalkylammonium fluoroborates, quaternary ammonia salts and mixtures thereof is used;
in step a) and/or b) the solvent is selected from aprotic solvents, protic solvent, polar aprotic solvents and mixtures thereof or is selected from polar aprotic solvents; or
in step a) an acid is used or wherein the acid used is selected from organic acids, sulfonic acids, organic polar acids and mixtures thereof;
in step a) the electrochemical oxidation is carried out at current densities selected from 1 to 50 A/m²;
in step b) the electrochemical reduction is carried out at current densities selected from 10 to 500 A/m².

In one embodiment,
the current density used in step a) is decreased or stepwise decreased;
current density used in step a) is lower than the current density used in step b);
step a) is conducted at temperatures selected from 20 to 120° C.,
in step a) and/or b) a conducting salt is used and the conducting salt selected from the group of organic ammonia salts, ionic liquids, tetraalkylammonium fluoroborates, quaternary ammonia salts and mixtures thereof is used;
in step a) and/or b) the solvent is selected from aprotic solvents, protic solvent, polar aprotic solvents and mixtures thereof or is selected from polar aprotic solvents; or
in step a) an acid is used or wherein the acid used is selected from organic acids, sulfonic acids, organic polar acids and mixtures thereof;
in step a) the electrochemical oxidation is carried out at current densities selected from 1 to 100 A/m²;
in step b) the electrochemical reduction is carried out at current densities selected from 10 to 1000 A/m².

In one embodiment,
the current density used in step a) is decreased or stepwise decreased;
current density used in step a) is lower than the current density used in step b);
step a) is conducted at temperatures selected from 15 to 150° C.,
in step a) and/or b) a conducting salt is used and the conducting salt selected from the group of organic ammonia salts, ionic liquids, tetraalkylammonium fluoroborates, quaternary ammonia salts and mixtures thereof is used;
in step a) and/or b) the solvent is selected from aprotic solvents, protic solvent, polar aprotic solvents and mixtures thereof or is selected from polar aprotic solvents; or
in step a) an acid is used or wherein the acid used is selected from organic acids, sulfonic acids, organic polar acids and mixtures thereof;
in step a) the electrochemical oxidation is carried out at current densities selected from 1 to 100 A/m²;
in step b) the electrochemical reduction is carried out at current densities selected from 1 to 1000 A/m²
in step b) the electrochemical reduction is carried out at a temperature from 1 to 50° C.

In one embodiment,
the current density used in step a) is decreased or stepwise decreased;
current density used in step a) is lower than the current density used in step b);
step a) is conducted at temperatures selected from 15 to 150° C., 15 to 120° C., and 20 to 100° C.;
optionally in step a) first the electrochemical oxidation is conducted and then the reaction mixture is heated; or wherein first the electrochemical oxidation is conducted and then the reaction mixture is heated to temperature selected from 50 to 150° C., 75 to 150° C., to 80 to 150° C., 80 to 140° C. and 90 to 120° C.;
in step a) and/or b) a conducting salt is used and the conducting salt selected from the group of organic ammonia salts, ionic liquids, tetraalkylammonium fluoroborates, quaternary ammonia salts and mixtures thereof is used; or selected from the group of tetraethylammonium tetrafluoroborate, tetrabutylammonium hexafluorophosphate, tetrabutylammonium hydrogen sulfate and mixtures thereof is used;
in step a) and/or b) the solvent is selected from
   aprotic solvents, protic solvent, and mixtures thereof or is selected from polar aprotic solvents; or
   polar aprotic solvents selected from dimethylformamide (DMF), dimethyl sulfoxide, acetonitrile, acetone; or
   protic solvents selected from methanol, acetic acid, ethanol, formic acid, water and mixtures thereof; or
   dimethylformamide (DMF), methanol, acetic acid and mixtures thereof; or
   methanol, acetic acid and mixtures thereof; or
   methanol; or
   acetic acid;
   or mixtures of the foregoing;
in step a) an acid is used or wherein the acid used is selected from
   organic acids, sulfonic acids, organic polar acids and mixtures thereof;
   acetic acid, formic acid and mixtures thereof is used;
   acetic acid;
   and mixtures of the foregoing;
in step a) the electrochemical oxidation is carried out at current densities selected from 1 to 5000 A/m², 1 to 500 A/m², 1 to 100 A/m², 1 to 50 A/m², 5 to 5000 A/m², 5 to 500 A/m², 5 to 100 A/m², 5 to 50 A/m², 10 to 5000 A/m², 10 to 500 A/m², 10 to 100 A/m², and 10 to 50 A/m²;
in step b) the electrochemical reduction is carried out at current densities selected from 1 to 10000 A/m², 1 to 1000 A/m², 1 to 500 A/m², 5 to 10000 A/m², 50 to 1000 A/m², 200 to 500 A/m², 10 to 10000 A/m², 10 to 1000 A/m², 100 to 500 A/m², 200 to 400 A/m², 200 A/m², 250 A/m², 300 A/m², 350 A/m² and 400 A/m²;
in step b) the electrochemical reduction is carried out at a temperature from 1 to 50° C., 10 to 35° C., 15 to 30° C., or at ambient temperature.

In one embodiment,
the current density used in step a) is decreased or stepwise decreased;
current density used in step a) is lower than the current density used in step b);
step a) is conducted at temperatures selected from 15 to 150° C., 15 to 120° C., and 20 to 100° C.;
optionally in step a) first the electrochemical oxidation is conducted and then the reaction mixture is heated; or wherein first the electrochemical oxidation is conducted and then the reaction mixture is heated to temperature selected from 50 to 150° C., 75 to 150° C., to 80 to 150° C., 80 to 140° C. and 90 to 120° C.;
in step a) and/or b) a conducting salt is used and the conducting salt selected from the group of organic ammonia salts, ionic liquids, tetraalkylammonium fluoroborates, quaternary ammonia salts and mixtures thereof is used; or selected from the group of tetraethylammonium tetrafluoroborate, tetrabutylammonium hexafluorophosphate, tetrabutylammonium hydrogen sulfate and mixtures thereof is used,
in step a) and/or b) the solvent is selected from
   aprotic solvents, protic solvent, and mixtures thereof or is selected from polar aprotic solvents; or
   polar aprotic solvents selected from dimethylformamide (DMF), dimethyl sulfoxide, acetonitrile, acetone; or
   protic solvents selected from methanol, acetic acid, ethanol, formic acid, water and mixtures thereof; or
   dimethylformamide (DMF), methanol, acetic acid and mixtures thereof; or
   methanol, acetic acid and mixtures thereof; or
   methanol; or
   acetic acid;
   or mixtures of the foregoing;
in step a) an acid is used or wherein the acid used is selected from
   organic acids, sulfonic acids, organic polar acids and mixtures thereof;
   acetic acid, formic acid and mixtures thereof is used;
   acetic acid;
   and mixtures of the foregoing;
in step a) the electrochemical oxidation is carried out at a cell voltage from 0.1 to 20 V or is less than 20 V;
in step a) the electrochemical oxidation is carried out at current densities selected from 1 to 5000 A/m², 1 to 500 A/m², 1 to 100 A/m², 1 to 50 A/m², 5 to 5000 A/m², 5 to 500 A/m², 5 to 100 A/m², 5 to 50 A/m², 10 to 5000 A/m², 10 to 500 A/m², 10 to 100 A/m², and 10 to 50 A/m²;
in step b) the electrochemical reduction is carried out at a cell voltage from 0.1 to 30 V or is less than 30 V;
in step b) the electrochemical reduction is carried out at current densities selected from 1 to 10000 A/m², 1 to 1000 A/m², 1 to 500 A/m², 5 to 10000 A/m², 50 to 1000 A/m², 200 to 500 A/m², 10 to 10000 A/m², 10 to 1000 A/m², 100 to 500 A/m², 200 to 400 A/m², 200 A/m², 250 A/m², 300 A/m², 350 A/m² and 400 A/m²;
in step b) the electrochemical reduction is carried out at a temperature from 1 to 50° C., 10 to 35° C., 15 to 30° C., or at ambient temperature.

In one embodiment of step a), the flow rate is decreased. In one embodiment of step a), the flow rate is increased. In one embodiment of step a), the flow rate is continuous. In one embodiment of step a), the flow rate is varying. In one embodiment of step a), the flow rate is 0.01 mL/min to 10000 L/min. In one embodiment of step a), the flow rate is 0.01 mL/min to 1000 L/min, 0.01 mL/min to 100 L/min, 0.1 m/min to 1000 L/min, 0.1 mL/min to 100 L/min, 0.1 m/min to 10 L/min, 0.1 mL/min to 1000 m/min, 0.1 m/min to 100 m/min, 0.1 m/min to 10 m/min, 0.1 m/min to 9 m/min, 0.1 m/min to 8 mL/min, 0.1 m/min to 7 m/min, 0.1 m/min to 6 m/min, 0.1 m/min to 5 mL/min, 0.1 m/min to 4 mL/min, 0.1 mL/min to 3 mL/min, 0.1 mL/min to 2 m/min, 0.1 mL/min to 1.5 m/min, 0.1 mL/min to 1 mL/min, 0.5 m/min to 100 L/min, 0.5 mL/min to 10 L/min, 0.5 m/min to 1000 mL/min, 0.5 m/min to 100 m/min, 0.5 mL/min to 10 m/min, 0.5 mL/min to 9 mL/min, 0.5 mL/min to 8 m/min, 0.5 m/min to 7 m/min, 0.5 m/min to 6 m/min, 0.5 m/min to 5 m/min, 0.5 m/min to 4 m/min, 0.5 m/min to 3 m/min, 0.5 m/min to 2 m/min, 0.5 m/min to 1.5 mL/min, 0.7 mL/min to 100 L/min, 0.7 mL/min to 10 L/min, 0.7 mL/min to 1000 mL/min, 0.7 m/min to 100 m/min, 0.7 m/min to 10 mL/min, 0.7 m/min to 9 mL/min, 0.7 m/min to 8 m/min, 0.7 m/min to 7 m/min, 0.7 m/min to 6 mL/min, 0.7 m/min to 5 mL/min, 0.7 m/min to 4 m/min, 0.7 m/min to 3 m/min, 0.7 m/min to 2 m/min, 0.7 m/min to 1.5 m/min, or 1 mL/min.

In one embodiment of step a), the concentration of the reactant is decreased, increased, continuous and/or varied. The reactant can be added in a continuous manner. In one embodiment of step a), the concentration of the conducting salt is decreased, increased, continuous and/or varied. The conducting salt can be added in a continuous manner.

In one embodiment of step b), the flow rate is decreased. In one embodiment of step b), the flow rate is increased. In one embodiment of step b), the flow rate is continuous. In one embodiment of step a), the flow rate is varying. In one embodiment of step b) the flow rate is 0.01 m/min to 10000 L/min, 0.01 m/min to 100 L/min, 0.1 m/min to 1000 L/min, 0.1 mL/min to 100 L/min, 0.1 m/min to 10 L/min, 0.1 m/min to 1000 m/min, 0.1 m/min to 100 mL/min, 0.1 m/min to 10 mL/min, 0.1 m/min to 9 m/min, 0.1 m/min to 8 m/min, 0.1 m/min to 7 m/min, 0.1 m/min to 6 m/min, 0.1 m/min to 5 m/min, 0.1 mL/min to 4 mL/min, 0.1 m/min to 3 mL/min, 0.1 mL/min to 2 m/min, 0.1 m/min to 1.5 m/min, 0.1 m/min to 1 m/min, 0.5 m/min to 100 L/min, 0.5 mL/min to 10 L/min, 0.5 mL/min to 1000 mL/min, 0.5 m/min to 100 mL/min, 0.5 mL/min to 10 mL/min, 0.5 mL/min to 9 mL/min, 0.5 mL/min to 8 mL/min, 0.5 mL/min to 7 mL/min, 0.5 mL/min to 6 mL/min, 0.5 mL/min to 5 mL/min, 0.5 mL/min to 4 mL/min, 0.5 mL/min to 3 mL/min, 0.5 m/min to 2 mL/min, 0.5 mL/min to 1.5 mL/min, 0.7 mL/min to 100 L/min, 0.7 mL/min to 10 L/min, 0.7 mL/min to 1000 mL/min, 0.7 mL/min to 100 mL/min, 0.7 mL/min to 10 mL/min, 0.7 mL/min to 9 mL/min, 0.7 mL/min to 8 mL/min, 0.7 mL/min to 7 mL/min, 0.7 mL/min to 6 mL/min, 0.7 mL/min to 5 mL/min, 0.7 mL/min to 4 mL/min, 0.7 mL/min to 3 mL/min, 0.7 mL/min to 2 mL/min, 0.7 mL/min to 1.5 mL/min, or 1 mL/min.

In one embodiment of step b), the concentration of the reactant is decreased, increased, continuous and/or varied. The reactant can be added in a continuous manner. In one embodiment of step b), the concentration of the conducting salt is decreased, increased, continuous and/or varied. The electrolyte can be added in a continuous manner.

The cycle times for each of the steps a), b) c) and/or d) can be individually adjusted. One or more of these steps can be performed multiple times, with one or more of the steps being performed less often or even only once. In one embodiment step a) is performed multiple times. In one embodiment step b) is performed multiple times. In one embodiment step c) is performed multiple times. In one embodiment step d) is performed multiple times.

In one embodiment of step a), a pressure of 0.1 to 10 bar is used. In this embodiment, the pressure can be decreasing, increasing, constant or varying. In one embodiment of step a), a pressure of 0.1 to 9 bar, 0.1 to 8 bar, 0.1 to 7 bar, 0.1 to 6 bar, 0.1 to 5 bar, 0.1 to 4 bar, 0.1 to 2 bar, 0.1 to 1 bar, 0.5 to 9 bar, 0.5 to 8 bar, 0.5 to 7 bar, 0.5 to 6 bar, 0.5 to 5 bar, 0.5 to 4 bar, 0.5 to 2 bar, 0.5 to 1 bar, 0.75 to 9 bar, 0.75 to 8 bar, 0.75 to 7 bar, 0.75 to 6 bar, 0.75 to 5 bar, 0.75 to 4 bar, 0.75 to 2 bar, 0.75 to 1 bar, 1 to 9 bar, 1 to 8 bar, 1 to 7 bar, 1 to 6 bar, 1 to 5 bar, 1 to 4 bar, 1 to 2 bar, 1 bar, atmospheric pressure to 9 bar, atmospheric pressure to 8 bar, atmospheric pressure to 7 bar, atmospheric pressure to 6 bar, atmospheric pressure to 5 bar, atmospheric pressure to 4 bar, atmospheric pressure to 2 bar, or atmospheric pressure is used. In this embodiment, the pressure can be decreasing, increasing, constant or varying. The atmospheric pressure is 1.01325 bar. This pressure may vary as known by the person skilled in the art.

In one embodiment of step b), a pressure of 0.1 to 10 bar is used. In this embodiment, the pressure can be decreasing, increasing, constant or varying. In one embodiment of step b), a pressure of 0.1 to 9 bar, 0.1 to 8 bar, 0.1 to 7 bar, 0.1 to 6 bar, 0.1 to 5 bar, 0.1 to 4 bar, 0.1 to 2 bar, 0.1 to 1 bar, 0.5 to 9 bar, 0.5 to 8 bar, 0.5 to 7 bar, 0.5 to 6 bar, 0.5 to 5 bar, 0.5 to 4 bar, 0.5 to 2 bar, 0.5 to 1 bar, 0.75 to 9 bar, 0.75 to 8 bar, 0.75 to 7 bar, 0.75 to 6 bar, 0.75 to 5 bar, 0.75 to 4 bar, 0.75 to 2 bar, 0.75 to 1 bar, 1 to 9 bar, 1 to 8 bar, 1 to 7 bar, 1 to 6 bar, 1 to 5 bar, 1 to 4 bar, 1 to 2 bar, 1 bar atmospheric pressure to 9 bar, atmospheric pressure to 8 bar, atmospheric pressure to 7 bar, atmospheric pressure to 6 bar, atmospheric pressure to 5 bar, atmospheric pressure to 4 bar, atmospheric pressure to 2 bar, or atmospheric pressure is used. In this embodiment, the pressure can be decreasing, increasing, constant or varying.

The usage of a pressure higher than 1 bar or ambient pressure in step a) and/or b) can lead to a better solubility of the starting material, solvent, conducting salt, acid, intermediates and/or products obtained.

### Further Aspects

A further aspect of the invention is a process for synthesizing (4S)-4-(4-cyano-2-methoxyphenyl)-5-ethoxy-2,8-dimethyl-1,4-dihydro-1,6-naphthyridine-3-carboxamide of formula (I)

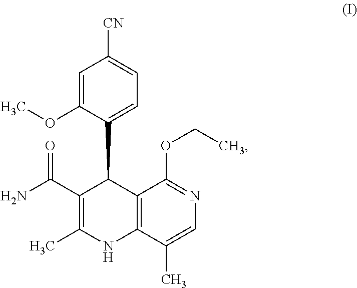 [SMILES: CCOc1ncc(C)c2c1[C@H](c1ccc(C#N)cc1OC)C(C(N)=O)=C(C)N2]

the synthesis comprising the steps of:
Step (1) a sequential one-pot synthesis for synthesizing 4-(4-cyano-2-methoxy-phenyl)-5-ethoxy-2,8-dimethyl-1,4-dihydro-1,6-naphthyridine-3-carboxamide according to formula (XIII)

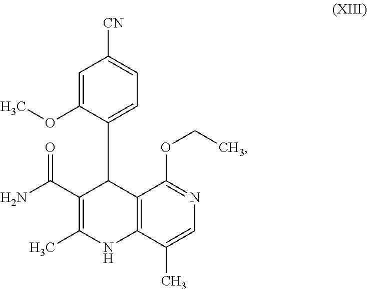 [SMILES: CCOc1ncc(C)c2c1C(c1ccc(C#N)cc1OC)C(C(N)=O)=C(C)N2]

the synthesis comprising steps a) and b):
step a) synthesizing 4-(4-cyano-2-methoxy-phenyl)-5-ethoxy-2,8-dimethyl-1,6-naphthyridine-3-carboxamide according to formula (XVII)

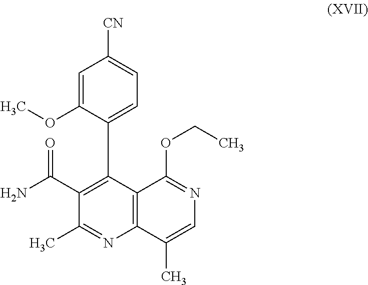 [SMILES: CCOc1ncc(C)c2nc(C)c(C(N)=O)c(-c3ccc(C#N)cc3OC)c12]

via electrochemical oxidation of (4R)-4-(4-cyano-2-methoxy-phenyl)-5-ethoxy-2,8-dimethyl-1,4-dihydro-1,6-naphthyridine-3-carboxamide according to formula ent-(I)

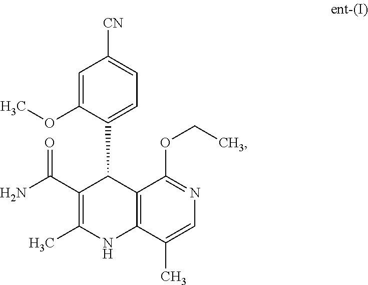 [SMILES: CCOc1ncc(C)c2c1[C@@H](c1ccc(C#N)cc1OC)C(C(N)=O)=C(C)N2]

step b) synthesizing 4-(4-cyano-2-methoxy-phenyl)-5-ethoxy-2,8-dimethyl-1,4-dihydro-1,6-naphthyridine-3-carboxamide according to formula (XIII)

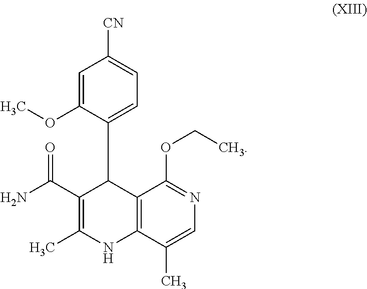 [SMILES: CCOc1ncc(C)c2c1C(c1ccc(C#N)cc1OC)C(C(N)=O)=C(C)N2]

via electrochemical reduction of the compound according to formula (XVII); and
Step (2) isolating the compound according to formula (I) from the compound according to formula (XIII).

Embodiments of step a) were already described above. These embodiments can be used in Step (1). Embodiments of step b) were already described above. These embodiments can be used in Step (1).

In one embodiment of step (1) the synthesis further comprises step c) isolation of the compound according to formula (XIII). Embodiments of step c) were already described above. These embodiments can be used in Step (1).

In one embodiment of step (1) the synthesis further comprises step d) crystallization of the compound according to formula (XIII). Embodiments of step d) were already described above. These embodiments can be used in Step (1).

In step (2), the compound according to formula (I) from the compound according to formula (XIII) is isolated. In one embodiment of step (2) isolation is selected from filtration, chiral chromatography, crystallization, racemic resolution, diastereomeric salt formation and chiral salt formation. For example, isolation via diastereomeric salt formation is described in US20100136142 A1, US20170217957 A1 or WO2019206909 A1.) In another aspect the sequential one-pot synthesis described here can be carried out several times in succession.

In one embodiment the sequential one-pot synthesis is carried out multiple times in succession. In one embodiment the sequential one-pot synthesis is carried out two times in succession. In one embodiment the sequential one-pot synthesis is carried out at least two times in succession. In one embodiment the sequential one-pot synthesis is carried out at least three times in succession. See e.g. scheme 5.

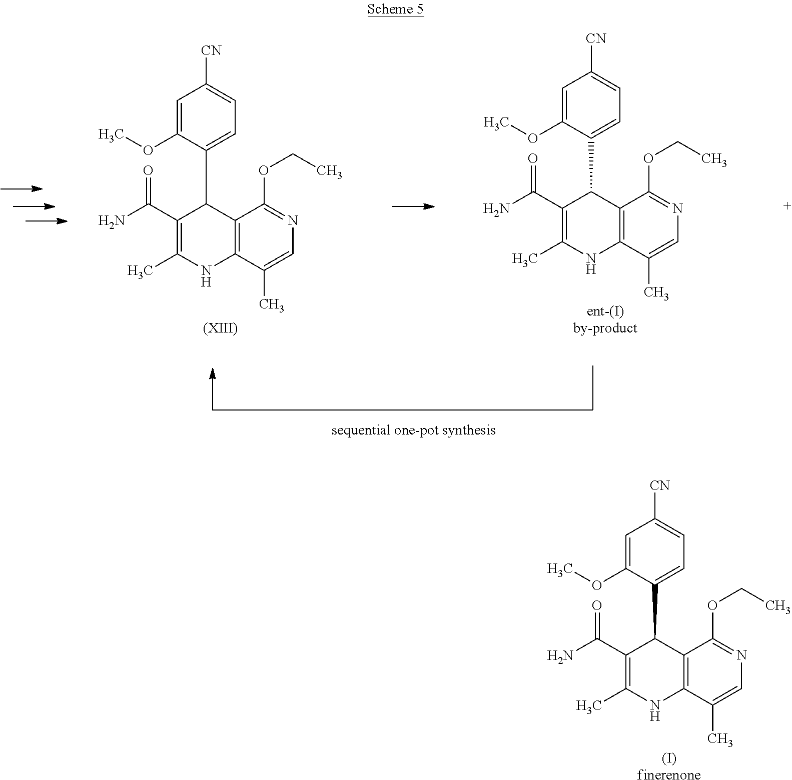

This offers the possibility of converting the compound ent-(I) into compound (XIII). This can be seen as a quasi-continuous mode of operation, which offers great advantages in terms of costs, time and/or resources. In this way, the waste product ent-(I) that occurs again and again in the preparation of finerenone (I) can be converted back into the compound (XIII). The compound (XIII) can then in turn be fed back into the production process of finerenone (I). After several process cycles of the sequential one-pot synthesis, the compound ent-(I) can, thus, be almost completely utilized. In the best case, the by-product ent-(I) can be almost completely recycled to the envisaged product (XIII) or finerenone (I).

In one embodiment, the process for the preparation of (4S)-4-(4-cyano-2-methoxyphenyl)-5-ethoxy-2,8-dimethyl-1,4-dihydro-1,6-naphthyridine-3-carboxamide of formula (I) is worked in a continuous mode, wherein
the starting material, solvent, conducting salt, acid, and/or intermediate is added continuously and
the compound according to formula (I) is removed continuously.

In one embodiment, (4S)-4-(4-cyano-2-methoxyphenyl)-5-ethoxy-2,8-dimethyl-1,4-dihydro-1,6-naphthyridine-3-carboxamide of formula (I) in step (1) and/or (2) is obtained in amount of 0.01 to 100 mm/mL, 0.1 to 50 mmol/L, 0.1 to 20 mmol/L, or 0.1 to 10 mmol/L, based on the total volume of the reaction mixture.

### FIGURES

FIG. 1 and FIG. 2 schematically show a set-up of an electrolytic apparatus.

FIG. 1: shows a set-up of an electrolysis apparatus. This set-up comprises an electrolysis cell (1), a power source (2), an electrolyte tank (3) and an anolyte tank (4). The arrows indicate the flow of the electrolyte and/or anolyte.

FIG. 2: shows a set-up of an electrolysis apparatus. This set-up comprises an electrolysis cell (1), a power source (2), an electrolyte tank (3), an anolyte tank (4) and pumps (5). It can optionally comprise one or more separators (6) and/or one or more heat exchangers (7). The arrows indicate the flow of the electrolyte and/or anolyte.

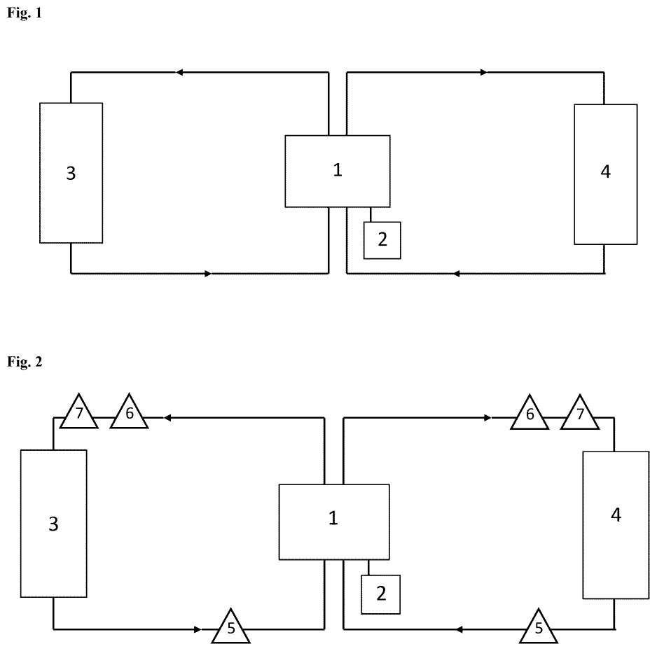

In FIGS. 1 and 2, further equipment can be integrated and/or adapted. For example, the electrolysis set-up can be adapted with respect to: electrode geometry, flow rate, dwell times, segments and so on. These aspects are described above. Further equipment can also be added: further pumps can be added, the set-up can be segmented, further pumps and/or tanks to e.g. provide the process with further starting material, solvent, conducting salt, acid, and/or intermediate, further equipment to remove or add the impurities, side-products, starting material, other reagents, solvents, intermediates and/or product, or to add further starting material, solvent, conducting salt, acid, and/or intermediate and/or product.

### EXPERIMENTAL PART

### A. Abbreviations and Acronyms

| EtOH | Ethanol |
|---|---|
| ° C. | degree Celsius |
| 1H-NMR | ¹H NMR Spectroscopy |
| A | Ampere |
| Ah | Ampere hour |
| cm² | square centimeter |
| approx. | approximately |
| DMF | dimethylformamide |
| Ethanol | Ethanol |
| | (contains 2% toluene (denatured)) |
| g | gramm |
| h | hour(s) |
| HPLC | High-pressure, high-performance liquid chromatography |
| IT | Inner temperature |
| kC | kilo Coulomb |
| kg | kilogram |
| L | liter |
| m2 | square meter |
| m3 | cubic meter |
| mbar | millibar |
| min | minute(s) |
| mL | milliliter |
| MS | mass spectrometry |
| RRT | Relative Retention Time |
| RT | Retention Time |
| TFA | CF₃COOH |
| TM | Jacket temperature |
| XRPD | X-ray powder diffraction |

Table 2 below gives the structures of the compounds found in HPLC. The HPLC retention time assignment is given below

TABELLE 2

| | |
|---|---|
| 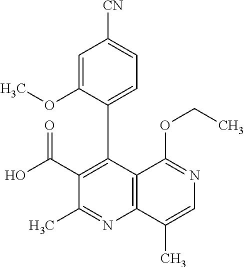 [SMILES: CCOc1ncc(C)c2nc(C)c(C(=O)O)c(-c3ccc(C#N)cc3OC)c12] | Impurity A |
| 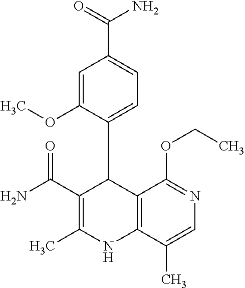 [SMILES: CCOc1ncc(C)c2c1C(c1ccc(C(N)=O)cc1OC)C(C(N)=O)=C(C)N2] | Impurity B |
| 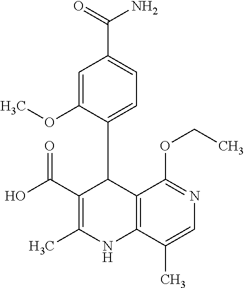 [SMILES: CCOc1ncc(C)c2c1C(c1ccc(C(N)=O)cc1OC)C(C(=O)O)=C(C)N2] | Impurity C |
| 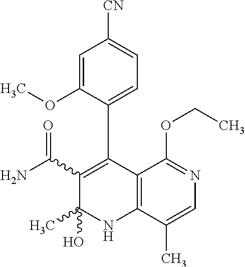 | Impurity D |
| 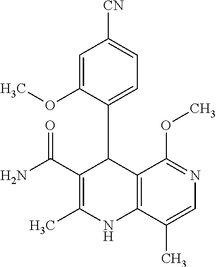 [SMILES: COc1cc(C#N)ccc1C1C(C(N)=O)=C(C)Nc2c(C)cnc(OC)c21] | Impurity E |
| 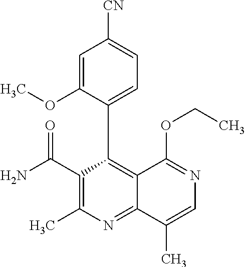 | Impurity F |
| 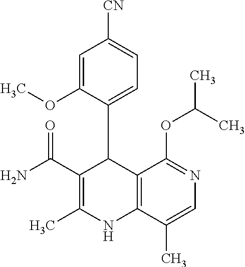 [SMILES: COc1cc(C#N)ccc1C1C(C(N)=O)=C(C)Nc2c(C)cnc(OC(C)C)c21] | Impurity G |
| 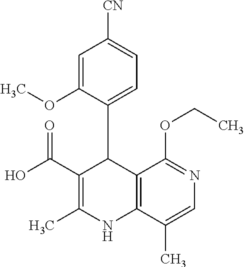 [SMILES: CCOc1ncc(C)c2c1C(c1ccc(C#N)cc1OC)C(C(=O)O)=C(C)N2] | Impurity H |
| 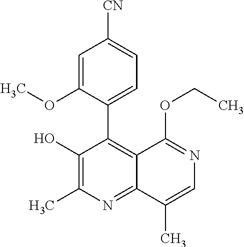 [SMILES: CCOc1ncc(C)c2nc(C)c(O)c(-c3ccc(C#N)cc3OC)c12] | Impurity K |
| 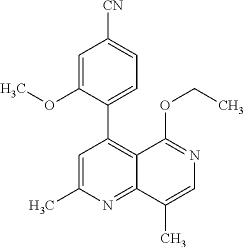 [SMILES: CCOc1ncc(C)c2nc(C)cc(-c3ccc(C#N)cc3OC)c12] | Impurity L |

### B. Analytical Methods

Analytical methods for testing organic impurities, content (assay) and enantiomeric purity at the 4-(4-cyano-2-methoxy-phenyl)-5-ethoxy-2,8-dimethyl-1,4-dihydro-1,6-naphthyridine-3-carboxamide (XIII) (crude and pure), (4S)-4-(4-cyano-2-methoxyphenyl)-5-ethoxy-2,8-dimethyl-1,4-dihydro-1,6-naphthyridine-3-carboxamide (I) (crude and pure) and (4R)-4-(4-cyano-2-methoxy-phenyl)-5-ethoxy-2,8-dimethyl-1,4-dihydro-1,6-naphthyridine-3-carboxamide (ent-(I)) levels.

### Method A (Assay and Organic Impurities)

| Devices | 1. | High performance liquid chromatograph with thermostatted column oven, UV | |
|---|---|---|---|
| | | detector and data evaluation system (e.g. Agilent 1260) | |
| | 2. | Metal column made of stainless steel | |
| | | Length: | 150 mm |
| | | inner diameter: | 4.6 mm |
| | | Particle size: | 3.0 um |
| | | Filling: | Hybride Silica-based ODS-C18-Phase |
| | | | (e.g .: Triart C18, YMC) |

| Reagents | 1. | Water for chromatography R |
|---|---|---|
| | 2. | Acetonitrile, e.g. Honeywell catalog number: 34967 (LC-MS grade, ≥99.9 %) |
| | 3. | Ammonium acetate, e.g. Sigma Aldrich, catalog number: 73594 (LC-MS grade, |
| | | ≥99.0%) |
| | 4. | Acetic acid, z.B. VWR catalog number: 184874.180 (LC-MS grade, ≥99.8 %) |
| Sample | Acetonitrile/Water = 1:1 (V/V) | |
| solvent | | |
| flow rate | 1.0 mL/min | |
| Eluent | A. | 10 mmol/L Ammonium acetate buffer pH 5.3 |
| | | e.g. dissolve 0.75 - 0.80 g Ammonium acetate and approx. 1.5 mL10% acetic |
| | | acid in 1000 mL water. pH is adjusted with 10% acetic acid to pH 5.3 ± 0.1. |
| | B. | Acetonitrile |

| | time [min] | A [%] | B [ %] |
|---|---|---|---|
| Gradient | 0.0 | 75 | 25 |
| | 3.0 | 75 | 25 |
| | 20.0 | 45 | 55 |
| | 28.0 | 25 | 75 |
| | 33.0 | 25 | 75 |
| | 33.1 | 75 | 25 |

| Running time of the chromatogram | 33.0 min | |
|---|---|---|
| Injection volume | 5.0 μL | |
| Sampler temperature | 23° C. | |
| Column oven temperature | 15° C. | |
| Detection measurement wavelength | 252 nm | |
| Retention time (Method A) | RT (min) | RRT |
| Main compound [compound (XIII)] | 14.9 | 1.00 |
| Impurity A | 7.4 | 0.49 |
| Impurity B | 7.7 | 0.52 |
| Impurity C | 9.7 | 0.65 |
| Impurity D | 9.9 | 0.66 |
| Impurity E | 12.6 | 0.85 |
| Impurity F | 16.5 | 1.11 |
| Impurity G | 17.1 | 1.15 |
| Impurity H | 17.9 | 1.20 |
| Impurity K | 24.9 | 1.68 |
| Impurity L | 26.9 | 1.79 |

The “main compound” is the compound according to formula (XIII), in particular the mixture of its enantiomers according to formula (I) and/or ent-(I).

### Method B (Assay and Organic Impurities)

| Devices | 1. | High performance liquid chromatograph with thermostatted column oven, UV | |
|---|---|---|---|
| | | detector and data evaluation system (e.g., Agilent 1260) | |
| | 2. | Metal column made of stainless steel | |
| | | Length: | 150 mm |
| | | inner diameter: | 4.6 mm |
| | | Particle size: | 3.0 um |
| | | Filling: | Hybride Silica-based ODS-C18-Phase |
| | | | (e.g ..: Triart C18, YMC) |

| Reagents | 1. | Acetonitrile, for HPLC |
|---|---|---|
| | 2. | CF3COOH, p.a. |
| | 3. | Water, p.a. |
| Sample | Acetonitrile/Water1:1 (v/v) | |
| solvent | | |
| Eluent | A | 0.1% CF₃COOH in Water |
| | | e.g., 1.0 mL CF₃COOH dissolved in 1000 mL water. |
| | B | Acetonitrile |

| | Time [min] | A [%] | B [ % ] |
|---|---|---|---|
| Gradient | 0.0 | 90 | 10 |
| | 2.0 | 90 | 10 |
| | 24.5 | 35 | 65 |
| | 26.0 | 20 | 80 |
| | 30.0 | 20 | 80 |

| Running time of the | 30.0 min | |
|---|---|---|
| chromatogram | | |
| Flow rate | 1.2 mL/min | |
| Volume of injection | 5 uL | |
| Column oven temperature | 20 ° C. | |
| Detection measurement | 252 nm | |
| wavelength | | |
| Retention time (Method B) | RT (min) | RRT |
| Main compound [compound | 10.8 | 1.00 |
| (XIII)] | | |
| Impurity B | 7.2 | 0.62 |
| Impurity D | 9.9 | 0.92 |
| Impurity E | 9.7 | 0.90 |
| Impurity G | 11.7 | 1.08 |
| Impurity H | 13.4 | 1.24 |
| Impurity L | 17.3 | 1.60 |
| Impurity F | 17.7 | 1.63 |
| Impurity A | 21.5 | 1.99 |

The “main compound” is the compound according to formula (XIII), in particular the mixture of its enantiomers according to formula (I) and/or ent-(I).

The HPLC analysis data given in the following examples regarding purity and content for 4-(4-cyano-2-methoxy-phenyl)-5-ethoxy-2,8-dimethyl-1,4-dihydro-1,6-naphthyridine-3-carboxamide (XIII) (crude and pure), (4S)-4-(4-cyano-2-methoxyphenyl)-5-ethoxy-2,8-dimethyl-1,4-dihydro-1,6-naphthyridine-3-carboxamide (I) (crude and pure) and (4R)-4-(4-cyano-2-methoxy-phenyl)-5-ethoxy-2,8-dimethyl-1,4-dihydro-1,6-naphthyridine-3-carboxamide (ent-(I)) relate only to impurities with more than 0.05% in the product. This is essentially impurity D. All other impurities shown in the table above are typically less than 0.05%. The structure of such impurities was determined by isolation and structure elucidation from enriched mother liquors.

### Method C (Enantiomeric Purity, Enantiomeric Excess e.e. %)

| Device/ | High-performance liquid chromatograph with thermostatically controlled column oven, UV | |
|---|---|---|
| Detector | detector and data evaluation system | |
| Detection | 252 nm | |
| measuremen | | |
| t wavelength | | |
| Column | 40° C. | |
| oven | | |
| temperature | | |
| Column | Chiralpak IC3 | |
| Length | 150 mm, inner diameter: 4.6 mm, Particle size: 3 μm | |
| Reagents: | 1. | Acetonitrile, e.g. Merck catalog number: 1.00030 (LiChrosolv® for HPLC |
| | | ≥99.9 %) |
| | 2. | Ammonium acetate, z. B. VWR catalog number: 153164R (HiPersolv |
| | | Chromanorm® HPLC grade ≥98.0 %) |
| | 3. | Water for Chromatography R |
| | 4. | Acetic acid, e.g. VWR catalog number: 184874.180 (HiPersolv Chromanorm™ for |
| | | LC-MS ≥99.8 %) |
| Eluent | A | 20 mmol/L Ammonium acetate buffer pH 5,0 (e.g. 1.50-1.60 g Dissolve ammonium |
| | | acetate and approx. 0.3 mL acetic acid in 1000 mL water. Adjust pH with 10% acetic |
| | | acid to pH 5.0 ± 0.1. |
| | B | Acetonitrile |

| | |
|---|---|
| Isocratic | Eluent A and Eluent B mix in a volume ratio of 1:1. |
| Elution | |
| Flow rate | 1 mL/min |
| Duration | 20 min |
| Sample | Eluent A and Eluent B mix in a volume ratio of 1:1. |
| solvent | |
| Injection | 6 μL |
| volume | |

| Retention time | RT (min) | RRT |
|---|---|---|
| (4S)-4-(4-cyano-2-methoxyphenyl)-5-ethoxy-2,8-dime | 8.2 | 1.00 |
| 1,4-dihydro-1,6-naphthyridine-3- carboxamide (I) | | |
| (4R)-4-(4-cyano-2-methoxy-phenyl)-5-ethoxy-2,8- | 9.5 | 1.16 |
| dimethyl-1,4-dihydro-1,6-naphthyridine-3-carboxamid | | |
| (ent-(I)) | | |

### C. Preparation Examples

### C.1 Preparation of 4-(4-cyano-2-methoxy-phenyl)-5-ethoxy-2,8-dimethyl-1,4-dihydro-1,6-naphthyridine-3-carboxamide (XIII)

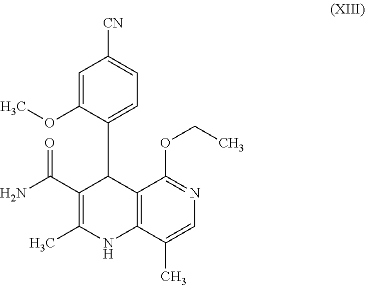 [SMILES: CCOc1ncc(C)c2c1C(c1ccc(C#N)cc1OC)C(C(N)=O)=C(C)N2]

Here, the sequential one-pot synthesis starting from ent-(I)

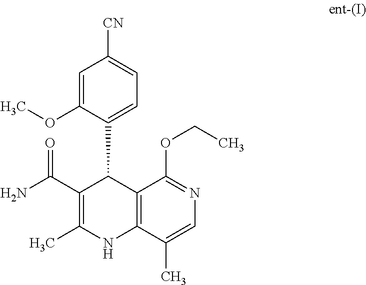 [SMILES: CCOc1ncc(C)c2c1[C@@H](c1ccc(C#N)cc1OC)C(C(N)=O)=C(C)N2]

comprising step a) and step b) is described.

### C.1.1 Example 1 (XIII), Example 2 (XIII) and Example 3 (XIII)

(4R)-4-(4-cyano-2-methoxy-phenyl)-5-ethoxy-2,8-dimethyl-1,4-dihydro-1,6-naphthyridine-3-carboxamide (ent-(I)) was dissolved in a mixture comprising DMF, methanol, tetraethylammonium tetrafluoroborate and acetic acid at room temperature in the catholyte tank (stirred tank). For the electrochemical oxidation of (4R)-4-(4-cyano-2-methoxy-phenyl)-5-ethoxy-2,8-dimethyl-1,4-dihydro-1,6-naphthyridine-3-carboxamide (ent-(I)) to 4-(4-cyano-2-methoxy-phenyl)-5-ethoxy-2,8-dimethyl-1,6-naphthyridine-3-carboxamide (XVII), this mixture was pumped out of the catholyte tank at a flow stream rate of 1.8 m³/h (Example 1 and Example 2) or 4.5 m³/h (Example 3), respectively, through an electrochemical cell (from the company Electrocell) with an anode and cathode area of 0.4 m² (Example 1 and Example 2) or 1.2 m² (Example 3), respectively.

The amounts of the components used for Examples 1 to 3 are summarized in table 3 below:

TABLE 3
Composition electrolyte solution (electrochemical oxidation)

| | Example 1 | Example 2 | Example 3 |
|---|---|---|---|
| (4R)-4-(4-cyano-2-methoxy- | 2.0 kg | 2.12 kg | 6.14 kg |
| phenyl)-5-ethoxy-2,8-dimethyl- | (5.29 mol) | (5.61 mol) | (16.24 mol) |
| 1,4-dihydro-1,6-naphthyridine- | | | |
| 3-carboxamide (ent-(I)) | | | |
| DMF | 21.7 kg | 29.3 kg | 85.0 kg |
| methanol | 5.42 kg | 1.55 kg | 2.50 kg |
| tetraethylammonium | 0.330 kg | 0.330 kg | 1.00 kg |
| tetrafluoroborate | | | |
| acetic acid | 0.54 kg | 0.54 kg | 1.50 kg |

The flow stream was divided roughly equally between anolyte and catholyte half-cells. After leaving the cell, the anolyte and catholyte volume flow streams were recombined and transferred back to the catholyte tank. During pumping, the electrolysis was started by supplying the cell with voltage and current via a rectifier. To achieve high chemical selectivity and simultaneously high current selectivity, the current density was gradually reduced with increasing conversion. A summary of the current density used in Examples 1 to 3 is summarized in table 4 below:

TABLE 4
Current densities (gradually reduced with increasing conversion) (electrochemical oxidation)

| | Example 1 | Example 2 | Example 3 |
|---|---|---|---|
| current | 340 min at 20A (50 A/m²) | 360 min at 20A (50 A/m²) | 348 min at 60A (50 A/m²) |
| density | 210 min at 16A (40 A/m²) | 225 min at 16A (40 A/m²) | 217 min at 48A (40 A/m²) |
| | 290 min at 12A (30 A/m²) | 310 min at 12A (30 A/m²) | 300 min at 36A (30 A/m²) |
| | 210 min at 8A (20 A/m²) | 225 min at 8A (20 A/m²) | 217 min at 24A (20 A/m²) |
| | 270 min at 4A (10 A/m²) | 320 min at 4A (10 A/m²) | 309 min at 12A (10 A/m²) |
| total | approx. 96.4% of the amount | approx. 97.6% of the amount | approx. 97.6% of the amount |
| amount | of charge theoretically | of charge theoretically | of charge theoretically |
| of | required for 100% | required for 100% | required for 100% |
| charge | conversion (1021 kC). | conversion (1083 kC). | conversion (3134 kC) |

Subsequently, the reaction solution was heated to a temperature of approx. 100° C. in the catholyte tank and stirred at this temperature for 16 hours under a reflux condenser.

The sequential one-pot synthesis was continued with the electrochemical reduction of 4-(4-cyano-2-methoxy-phenyl)-5-ethoxy-2,8-dimethyl-1,6-naphthyridine-3-carboxamide (XVII) to 4-(4-cyano-2-methoxy-phenyl)-5-ethoxy-2,8-dimethyl-1,4-dihydro-1,6-naphthyridine-3-carboxamide (XIII). A second tank (anolyte tank (stirred tank)), filled with a mixture of DMF, tetraethylammonium tetrafluoroborate, acetic acid and methanol was connected to the electrochemical cell.

The amounts of the components used for Examples 1 to 3 are summarized in table 5 below:

TABLE 5
Composition anolyte solution (electrochemical reduction)

| | Example 1 | Example 2 | Example 3 |
|---|---|---|---|
| DMF | 93.0 kg | 94.4 kg | 200.1 kg |
| tetraethylammonium | 1.43 kg | 1.25 kg | 2.50 kg |
| tetrafluoroborate | | | |
| acetic acid | 2.32 kg | 2.05 kg | 3.80 kg |
| methanol | 23.2 kg | 16.6 kg | 22.2 kg |

This anolyte solution was pumped at 0.9 m³/h from the tank through the anolyte half-cell of a divided electrochemical cell (from Electrocell) with an anode and cathode area of 0.4 m² (Example 1 and Example 2) or 1.2 m² (Example 3). At the same time, the solution obtained by electrochemical oxidation was pumped from the catholyte tank with the same flow volume (0.9 m³/h) through the catholyte half-cell of the electrochemical cell. After leaving the cell, the anolyte and catholyte volume flow streams were each returned separately to their outlet tank. During pumping, the electrolysis was started by supplying the cell with voltage and current via a rectifier. A constant current density of 350 A/m² was used and the electrolysis was terminated after 20 hours (Example 1), 6 hours (Example 2) or 7 hours (Example 3).

### C.2 Isolation and Purification of Examples 1 to 3 (XIII) [Crude]

In this section, the isolation of the compound according to formula (XIII)

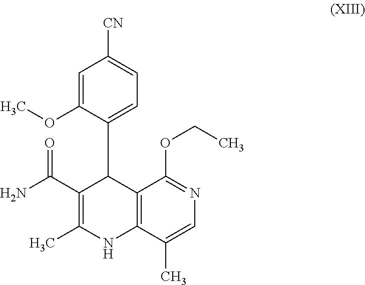 [SMILES: CCOc1ncc(C)c2c1C(c1ccc(C#N)cc1OC)C(C(N)=O)=C(C)N2]

is described (cf. step c)).

### C.2.1 Isolation and Purification of Examples 1 to 3 (XIII) [crude]

The total amount obtained from for Example 1, Example 2 and Example 3 after the end of the electrolysis from the catholyte cycle, including the volume from the catholyte tank, was further processed to obtain 4-(4-cyano-2-methoxy-phenyl)-5-ethoxy-2,8-dimethyl-1,4-dihydro-1,6-naphthyridine-3-carboxamide (XIII) as isolated solid as follows:

The product solution of the electrochemical reduction was concentrated by vacuum distillation at temperatures of up to 60° C. or 70° C. After the vacuum distillation was finished, (purified) water was added to the solution at 60° C. over 3 or 4 hours to induce crystallization. The suspension was then cooled to room temperature, stirred for further 2 hours and the product was filtered and washed with water. The product obtained was then dried 55° C. in a drying cabinet (under vacuum).

The yield obtained is summarized in table 6 below:

TABLE 6
Overview yield of 4-(4-cyano-2-methoxy-phenyl)-5-ethoxy-2,8-
dimethyl-1,4-dihydro-1,6-naphthyridine-3-carboxamide (XIII)
(Example 1, Example 2 and Example 3, crude)

| | Example 1 | Example 2 | Example 3 |
|---|---|---|---|
| % of theory | 81.5% | 75.3% | 80.8% |
| Purity | more than 99% | 99.9% | 99.8% |
| Method | B | B | B |

### C.2.2 Example 1 (XIII) [Pure]

In this section an example for step d) is described:

Approx. 1000 g of crude Example 1 were suspended in a 1:1 v/v mixture of isopropanol and n-propanol at room temperature (approx. 6% by weight compound (XIII) [crude, Example 1]). The solid was first completely dissolved by heating to above 85° C. (slight reflux) and then cooled to 5° C. by cooling the solution (in 3 h) and stirring was continued overnight. During the cooling process, the crystallization of the pure form of compound (XIII) begins. The solid was filtered (using a centrifuge), the reactor was rinsed with 2 L of isopropanol at 5° C. and the wet cake obtained in this way was washed with pre-cooled (5° C.) isopropanol (2 L). After drying at 55° C. in a drying cabinet (under vacuum), >890 g of pure compound (XIII) were obtained:

Yield: 89% (of theory)

### Analytical Results:

Assay % compound (XIII) (HPLC-Method A): 100.3%
Area % main compound [compound (XIII)](HPLC-Method A): 99.87%
Analysis of the enantiomeric ratio gave the expected values for a racemate of ~50:50 (49.6:50.4), corresponds to e.e.%: 0.8%. (HPLC-Method C)

### C.3 Example 4—Preparation of (4S)-4-(4-cyano-2-methoxyphenyl)-5-ethoxy-2,8-dimethyl-1,4-dihydro-1,6-naphthyridine-3-carboxamide (I)

This example describes how finerenone (1)

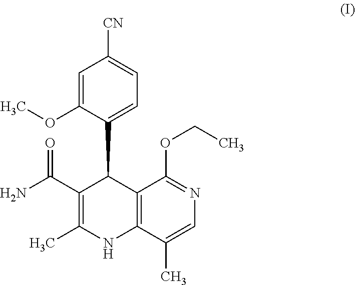 [SMILES: CCOc1ncc(C)c2c1[C@H](c1ccc(C#N)cc1OC)C(C(N)=O)=C(C)N2]

was obtained from recycled 4-(4-cyano-2-methoxy-phenyl)-5-ethoxy-2,8-dimethyl-1,4-dihydro-1,6-naphthyridine-3-carboxamide (XIII).

4-(4-cyano-2-methoxy-phenyl)-5-ethoxy-2,8-dimethyl-1,4-dihydro-1,6-naphthyridine-3-carboxamide (XIII) obtained as described above was separated into the enantiomers (ent-(I) and (I)) either by SMB or resolution of diasteromeric salt formation (cf. section C.3.1, (4S)-4-(4-cyano-2-methoxyphenyl)-5-ethoxy-2,8-dimethyl-1,4-dihydro-1,6-naphthyridine-3-carboxamide-dibenzoyl-tartrate (II))—as described in for example US20100136142 A1, US20170217957 A1 or WO 2019206909 A1.

(4S)-4-(4-cyano-2-methoxyphenyl)-5-ethoxy-2,8-dimethyl-1,4-dihydro-1,6-naphthyridine-3-carboxamide (I) obtained after separation, is then purified by crystallization from ethanol (with 2% toluene) to yield (4S)-4-(4-cyano-2-methoxyphenyl)-5-ethoxy-2,8-dimethyl-1,4-dihydro-1,6-naphthyridine-3-carboxamide (I) in an excellent purity.

### C.3.1 (4S)-4-(4-cyano-2-methoxynhenyl)-5-ethoxy-2,8-dimethyl-1,4-dihydro-1,6-nanhthyridine-3-carboxamide-dibenzoyl-tartrate (II)

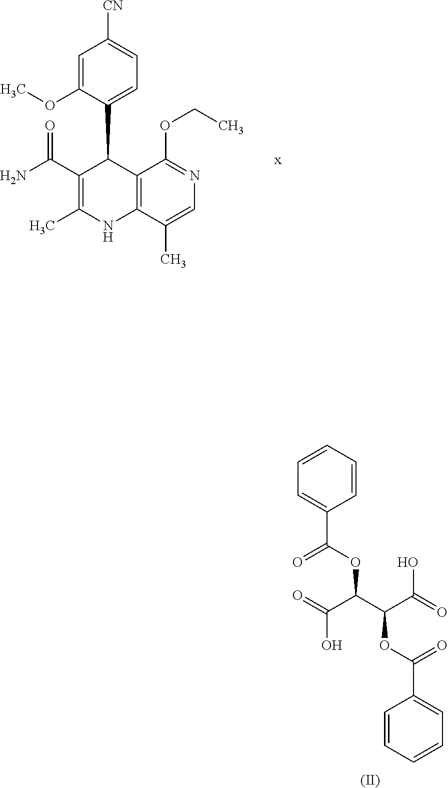

1.095 kg (2.90 mol) of racemic mixture 4-(4-cyano-2-methoxy-phenyl)-5-ethoxy-2,8-dimethyl-1,4-dihydro-1,6-naphthyridine-3-carboxamide (XIII) were dissolved at room temperature (approx. 23° C.) in 13.826 kg of a mixture consisting of ethanol (2% toluene)/water=75:25 (v/v) [9.719 kg/4.107 kg]. To this solution 0.570 kg (1.59 mol) (+)-O,O-dibenzoyl-D-tartaric acid were added and then approx. 3 kg of a solvent mixture consisting of ethanol (2% toluene)/water=75:25 (v/v) to rinse the equipment. The suspension obtained in this way was heated to an internal temperature of 75° C. within 60 minutes and then stirred at this temperature for 3.0 hours. The mixture was then cooled to 23° C. via a cooling ramp over 5.0 hours and stirred at this temperature overnight (about 16 hours). The suspension was filtered and washed once with 1.681 kg of a mixture consisting of ethanol (denatured with 2% toluene)/water=75:25 (v/v) and pressed dry for about 45 minutes.

Wet cake yield: 1.66 kg. The wet product was then dried at 50° C. under reduced pressure (<100 mbar) to constant mass (reached after 17 hours).

Yield of (4S)-4-(4-cyano-2-methoxyphenyl)-5-ethoxy-2,8-dimethyl-1,4-dihydro-1,6-naphthyridine-3-carboxamide-dibenzoyl-tartrate (II): 1.106 kg (103.7% of theory) of a white powder.

Mother liquor: 14.98 kg of mother liquor contained max. 0.548 kg (1.45 mol) of theoretical amount of (4R)-4-(4-cyano-2-methoxy-phenyl)-5-ethoxy-2,8-dimethyl-1,4-dihydro-1,6-naphthyridine-3-carboxamide (ent-(I)) which can be used for further recycling.

### C.3.2 (4S)-4-(4-cyano-2-methoxyphenyl)-5-ethoxy-2,8-dimethyl-1,4-dihydro-1,6-naphthyridine-3-carboxamide (I) [crude]

1.104 kg of (4S)-4-(4-cyano-2-methoxyphenyl)-5-ethoxy-2,8-dimethyl-1,4-dihydro-1,6-naphthyridine-3-carboxamide-dibenzoyl-tartrate (II) (from section C.3.1) were suspended at room temperature (approx. 23° C.) in a mixture of ethanol (denatured with 2% toluene) (1.766 kg) and water (8.722 kg). The internal temperature was increased to 50° C. within one hour and then 4.416 kg of an aqueous 9.1% (weight) trisodium phosphate solution was added within 30 minutes (pH increase from pH 5.4 to 7.2). The mixture was stirred at this temperature for a further 3.0 hours (pH=7.5). The mixture was then cooled to 23° C. within two hours and stirred at this temperature overnight (about 14 hours). The white suspension was filtered and then washed once with a solvent mixture of 0.186 kg of ethanol (denatured with 2% toluene) and 0.883 kg of deionized water and then twice with 0.883 kg of deionized water each time. Wet cake weight: 0.840 kg. The wet product was resuspended in a mixture of 3.842 kg of ethanol (2% toluene) and 1.280 kg of deionized water at 23° C. The suspension is heated to 70° C. within 2 hours by using a ramp, and the resulting solution was stirred at 70° C. for 15 minutes. The pH is adjusted to pH 8.5 to 9.0 (final pH=8.75) with an aqueous 0.15% (weight) trisodium phosphate solution. Then 11.04 kg of deionized water were added to the solution at 70° C. within 30 to 60 minutes. The white, easily stirrable suspension is cooled to 23° C. within 3 hours by means of a ramp and stirred for at least 1 hour. After stirring at 23° C. overnight, the suspension was filtered and then washed with 1.987 kg of deionized water and pressed dry for about 30 minutes. Wet cake weight: 0.571 kg. The moist product was then dried at 50° C. under reduced pressure (<100 mbar) to constant mass (reached after 17 hours).

Yield: 0.504 kg (88.9% of theory) white crystalline powder

### Analytical Results:

Double determination: assay % (4S)-4-(4-cyano-2-methoxyphenyl)-5-ethoxy-2,8-dimethyl-1,4-dihydro-1,6-naphthyridine-3-carboxamide (I), crude: (HPLC-Method B) 99.88% and 100.69%
Area % main compound [compound (XIII)](HPLC-Method B): 99.79%
Enantiomeric excess e.e. %: 99.76% (HPLC-Method C)

### C.3.3 (4S)-4-(4-cyano-2-methoxynhenyl)-5-ethoxy-2,8-dimethyl-1,4-dihydro-1,6-naphthyridine-3-carboxamide (I) [pure]

500 g of the crude (4S)-4-(4-cyano-2-methoxyphenyl)-5-ethoxy-2,8-dimethyl-1,4-dihydro-1,6-naphthyridine-3-carboxamide (I) (obtained in section C.3.2), were suspended in 8.5 L of ethanol (denatured with 2% toluene) and the mixture was then heated to reflux (78° C.). The product went into solution. It was stirred at this temperature for 45 minutes. The solution was filtered through a heated pressure filter, and the pressure filter was then rinsed with 0.5 L of ethanol (denatured with 2% toluene). The solvent was then distilled off until a final volume of 2.5 L was reached. The internal temperature was then cooled to 0° C. (ramp: duration approx. 4 hours) and stirring was continued at 0° C. for at least one hour (stirred over the weekend, but not necessary). The product was filtered off and washed twice with 0.5 L of ethanol (denatured with 2% toluene). Wet cake yield: 0.50 kg. The wet product was then dried at 50° C. under reduced pressure (<100 mbar) to constant mass (reached after 17 hours).

### Analytical Results:

Yield: 0.440 kg (88.0% of theory) white crystalline powder
Identity: structure verification was performed by 1H-NMR corresponds to published data
Assay % (4S)-4-(4-cyano-2-methoxyphenyl)-5-ethoxy-2,8-dimethyl-1,4-dihydro-1,6-naphthyridine-3-carboxamide (I) (HPLC-Method A): 99.91%
Area % main compound [compound (XIII)](HPLC-Method A): 100.0%
Enantiomeric excess e.e. %=100.0% (HPLC-Method C)
Residual solvent Ethanol: 0.05453 weight %

### C.4 Example 5 (XIII)

In this example, the recovery of compound ent-(I) from mother liquor and washing solution (section C.3, C3.1), the sequential one-pot synthesis starting from ent-(I) comprising step a), b) (section C.1) and step c) (section C.2) is described.

### C.4.1 Recovery of (4R)-4-(4-cyano-2-methoxy-phenyl)-5-ethoxy-2,8-dimethyl-1,4-dihydro-1,6-naphthyridine-3-carboxamide (ent-(I)) (from Mother Liquor and Washing Solutions)

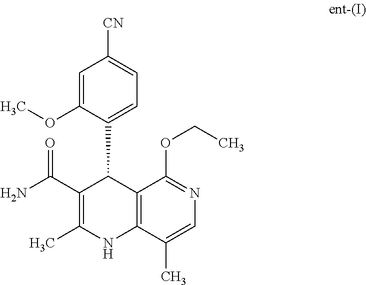 [SMILES: CCOc1ncc(C)c2c1[C@@H](c1ccc(C#N)cc1OC)C(C(N)=O)=C(C)N2]

(4R)-4-(4-cyano-2-methoxy-phenyl)-5-ethoxy-2,8-dimethyl-1,4-dihydro-1,6-naphthyridine-3-carboxamide (ent-(I)) obtained in the mother liquor in section C.3.1 comprising 14.98 kg (16.8 L) mother and wash liquor (corresponds to 0.548 kg of (4R)-4-(4-cyano-2-methoxy-phenyl)-5-ethoxy-2,8-dimethyl-1,4-dihydro-1,6-naphthyridine-3-carboxamide (ent-(I))). The pH of this solution was adjusted to pH 7.0-7.5 by using an aqueous Na₃PO₄ solution. The mixture was then concentrated in vacuum (40-85 mbar) to a residual volume of about 4.2 L (about 12.6 L of distillate), resulting in a white, easily stirrable suspension. At approx. 23+/−3° C., 8.4 L of deionized water were added within 15 minutes. After stirring for at least 2 hours (stirred overnight for organizational reasons), the mixture was filtered through a frit, the vessel was rinsed with mother liquor and the filter cake obtained was washed with 2.3 kg of deionized water and then pressed dry. Wet cake yield: 1.07 kg. The wet product was then dried at 50° C. under reduced pressure (<100 mbar) to constant mass (reached after 17 hours).

Yield: 0.507 kg (92.5% of theory) white crystalline powder.

### Analytical Results:

Assay % (4R)-4-(4-cyano-2-methoxy-phenyl)-5-ethoxy-2,8-dimethyl-1,4-dihydro-1,6-naphthyridine-3-carboxamide (ent-(I)) (HPLC-Method B): 100.1%
Area % main compound [compound (XIII)](HPLC-Method B): 99.66%
Enantiomeric excess e.e. %=97.88% (HPLC-Method C)

### C.4.2 Example 5 (XIII) [Crude]

(4R)-4-(4-cyano-2-methoxy-phenyl)-5-ethoxy-2,8-dimethyl-1,4-dihydro-1,6-naphthyridine-3-carboxamide (ent-(I)) obtained as described above was converted back to the racemic (4-(4-cyano-2-methoxy-phenyl)-5-ethoxy-2,8-dimethyl-1,4-dihydro-1,6-naphthyridine-3-carboxamide (XIII) and finally crystallized as a solid. The sequential one-pot synthesis was carried out on a laboratory scale (starting material: 50.4 g (4R)-4-(4-cyano-2-methoxy-phenyl)-5-ethoxy-2,8-dimethyl-1,4-dihydro-1,6-naphthyridine-3-carboxamide (ent-(I))), a total of 149.0 g 4-(4-cyano-2-methoxy-phenyl)-5-ethoxy-2,8-dimethyl-1,4-dihydro-1,6-naphthyridine-3-carboxamide (XIII) being obtained in 4 batches. A laboratory approach is described in detail below as an example:

50.4 g (0.133 mol) of (4R)-4-(4-cyano-2-methoxy-phenyl)-5-ethoxy-2,8-dimethyl-1,4-dihydro-1,6-naphthyridine-3-carboxamide (ent-(I)) were dissolved at room temperature in a mixture comprising 542.2 g of DMF, 135.6 g of methanol, 8.3 g of tetraethylammonium tetrafluoroborate and 13.6 g of acetic acid. For the electrochemical oxidation of (4R)-4-(4-cyano-2-methoxy-phenyl)-5-ethoxy-2,8-dimethyl-1,4-dihydro-1,6-naphthyridine-3-carboxamide (ent-(I)) to compound (XVII), this mixture was pumped at a total of 50 kg/h from a container (5 L) through an electrochemical laboratory cell (Multipurpose Cell, MPC) from the company Electrocell with an anode and cathode area of 100 cm². The flow stream was divided roughly equally between anolyte and catholyte half-cells. After leaving the cell, the anolyte and catholyte volume flow streams were combined and conveyed back to the container (in this way a circulation is performed). During pumping, the electrolysis was started by supplying the cell with voltage and current via a rectifier. To achieve high chemical selectivity and simultaneously high current selectivity, the current density was gradually reduced with increasing conversion:
340 min at 0.5 A (50 A/m²)
210 min at 0.4 A (40 A/m²)
210 min at 0.3 A (30 A/m²)
210 min at 0.2 A (20 A/m²)
510 min at 0.1 A (10 A/m²)

The total amount of charge that has flowed (6.833 Ah) corresponds to approx. 95.6% of the amount of charge theoretically necessary for 100% conversion (7.147 Ah). Subsequently, the reaction solution was heated to a temperature of about 100° C. in a 2 L multi-necked flask and stirred at this temperature for a total of 16 h (2×8 h) using under a reflux condenser. For the following electrochemical reduction of to 4-(4-cyano-2-methoxy-phenyl)-5-ethoxy-2,8-dimethyl-1,4-dihydro-1,6-naphthyridine-3-carboxamide (XIII), another container was used (anolyte reservoir, 5 L) which was filled with a solution of 2236.9 g DMF, 33.6 g tetraethylammonium tetrafluoroborate, 55.9 g acetic acid and 559.2 g of methanol. The solution prepared in this way was pumped with a volume stream of 25 kg/h from the container through the anolyte half-cell of a divided electrochemical laboratory cell (Multipurpose Cell, MPC) from Electrocell with an anode and cathode area of 100 cm2. At the same time, the solution obtained in step 2 was pumped from the catholyte reservoir (5-liter bottle) with the same delivery volume (25 kg/h) through the catholyte half-cell of the electrochemical cell. After leaving the cell, the anolyte and catholyte volume flows were each returned separately to their starting container (two circuits: anolyte and catholyte circuit). During pumping, the electrolysis was started by supplying the cell with voltage and current via a rectifier. In this partial step, a constant current density of 350 A/m² was used and the electrolysis was terminated after 20 hours. The total amount obtained from the catholyte cycle after the end of the electrolysis was used for the final product isolation: For the crystallization of crude 4-(4-cyano-2-methoxy-phenyl)-5-ethoxy-2,8-dimethyl-1,4-dihydro-1,6-naphthyridine-3-carboxamide (XIII), the product solution of the electrochemical reduction was concentrated by vacuum distillation at temperatures of up to 70° C. (4-(4-cyano-2-methoxy-phenyl)-5-ethoxy-2,8-dimethyl-1,4-dihydro-1,6-naphthyridine-3-carboxamide (XIII) was increased to 10-14% by weight, with the product was still dissolved at a reactor temperature of 60° C.). After the vacuum was finished, (purified) water was added to the solution at 60° C. over 4 hours to induce crystallization. After all the water had been added (about 4:1 v/v water: concentrate), the suspension was cooled to room temperature, stirred for a further 2 h and the product was filtered and washed with water. After drying at 55° C. in a drying cabinet (under vacuum), 39.2 g of crude 4-(4-cyano-2-methoxy-phenyl)-5-ethoxy-2,8-dimethyl-1,4-dihydro-1,6-naphthyridine-3-carboxamide (XIII) were obtained.

Yield: 39.2 g (77.8% of theory) off-white crystalline powder.

### Analytical Results:

Assay % 4-(4-cyano-2-methoxy-phenyl)-5-ethoxy-2,8-dimethyl-1,4-dihydro-1,6-naphthyridine-3-carboxamide (XIII) (HPLC-Method B) 98.1%
Area % main compound [compound (XIII)](HPLC-Method B): 98.77%
Enantiomeric excess e.e. %=0.88% (HPLC-Method C)

### C.4.3 Example 4 (XIII) [Pure]

70 g of the racemate obtained in this way (crude 4-(4-cyano-2-methoxy-phenyl)-5-ethoxy-2,8-dimethyl-1,4-dihydro-1,6-naphthyridine-3-carboxamide (XIII)) were suspended in a 1:1 w/w mixture of isopropanol and n-propanol (591.1 g each) at room temperature in a 2-liter vessel. The solid was first completely dissolved by heating to over 85° C. (slight reflux) and the 4-(4-cyano-2-methoxy-phenyl)-5-ethoxy-2,8-dimethyl-1,4-dihydro-1,6-naphthyridine-3-carboxamide (XIII) was then crystallized again by cooling the solution (in 3 h) to 5° C. It was stirred overnight. The solid was filtered and the wet cake obtained in this way was washed with pre-cooled (5° C.) isopropanol (2×110 g). After drying at 50° C. in a drying cabinet (under vacuum)
Yield: 58.1 g (83% of theory) of 4-(4-cyano-2-methoxy-phenyl)-5-ethoxy-2,8-dimethyl-1,4-dihydro-1,6-naphthyridine-3-carboxamide (XIII) were obtained as a white crystalline powder.

### Analytical Results:

Assay % 4-(4-cyano-2-methoxy-phenyl)-5-ethoxy-2,8-dimethyl-1,4-dihydro-1,6-naphthyridine-3-carboxamide (XIII) (HPLC-Method B): 98.0%
Area % main compound [compound (XIII)](HPLC-Method B): 99.31%
Enantiomeric excess e.e. %=1.04% (HPLC-Method C)

### D. Summary Results

In section C.1 the synthesis of the compound according to formula (XIII) via sequential one-pot synthesis (steps a) and b)) was described. In section C.2.1 (step c)) and C.2.2 (step d)) the further purification of the compound according to formula (XIII) is described, respectively.

From the experiments it could be shown that with the sequential one-pot synthesis, it has been made possible to produce the compound according to formula (XIII) in a very efficient manner, in high yields, in large scale and in high purity. The sequential one-pot synthesis offers significant advantages over the prior art in terms of scalability and technical implementation. The overall yield is significantly higher compared to the previously described synthesis as summarized in table 7 below.

TABLE 7
Yields obtained from US 15/753,406 A1 (US'406) and the sequential one-pot synthesis

| | Yield (%) | Yield (%) | Yield (%) | |
|---|---|---|---|---|
| | after oxidation | after racemization | after reduction | Overall yield |
| | and isolation | and isolation | and isolation | (% of theory) |
| US'406 A1 | 86.7 % | 85.9 % | 85 % | 64 % |
| | of theory | of theory | of theory | of theory |
| Example in | Example 12b | Example 12b | Example 32 | isolated yield |
| US'406 | (HNO₃ oxidation) | | | |
| Sequential | — | — | — | Example 1: 81.5 % |
| one-pot | | | | Example 2: 75.3 % |
| synthesis | | | | Example 3: 80.8 % |

It is apparent from table 7 above that the overall yield of the sequential one-pot synthesis shows a significant higher overall yield (Example 1: 81.5%; Example 2: 75.3%; Example 3: 80.8%) than the synthesis described in U.S. Ser. No. 15/753,406 A1 (64%). This is also surprising since the sequential one-pot synthesis is a large-scale process. The skilled person would not have expected this result.

In section C.3 it is described how finerenone (I) was obtained from recycled 4-(4-cyano-2-methoxy-phenyl)-5-ethoxy-2,8-dimethyl-1,4-dihydro-1,6-naphthyridine-3-carboxamide (XIII). In section C.4, the recovery of compound ent-(I) from mother liquor and washing solution (section C.3, C3.1), the sequential one-pot synthesis starting from ent-(I) comprising step a), b) (section C.1) and step c) (section C.2) is described.

It was demonstrated that the sequential one-pot synthesis can be carried out several times in succession and, thus, offers the possibility of converting the compound ent-(I) into compound (XIII). This can be seen as a quasi-continuous mode of operation, which offers great advantages in terms of costs, time and/or resources. In this way, the waste product ent-(I) that occurs again and again in the preparation of finerenone (I) can be converted back into the compound (XIII). The compound (XIII) can then in turn be fed back into the production process of finerenone (I). After several process cycles of the sequential one-pot synthesis, the compound ent-(I) can, thus, be almost completely utilized. In the best case, the by-product ent-(I) can be almost completely recycled to the envisaged product (XIII) or finerenone (I). See e.g. scheme 4 above. Moreover, finerenone (I) could be obtained in a high purity.

1. A sequential one-pot synthesis for synthesizing 4-(4-cyano-2-methoxy-phenyl)-5-ethoxy-2,8-dimethyl-1,4-dihydro-1,6-naphthyridine-3-carboxamide according to formula (XIII) 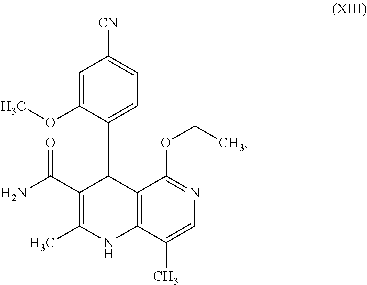
the synthesis comprising the steps of:
step a) synthesizing 4-(4-cyano-2-methoxy-phenyl)-5-ethoxy-2,8-dimethyl-1,6-naphthyridine-3-carboxamide according to formula (XVII) 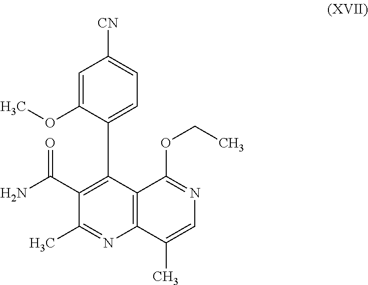
via electrochemical oxidation of (4R)-4-(4-cyano-2-methoxy-phenyl)-5-ethoxy-2,8-dimethyl-1,4-dihydro-1,6-naphthyridine-3-carboxamide according to formula ent-(I) 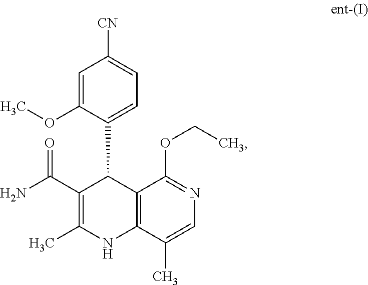
step b) synthesizing 4-(4-cyano-2-methoxy-phenyl)-5-ethoxy-2,8-dimethyl-1,4-dihydro-1,6-naphthyridine-3-carboxamide according to formula (XIII) 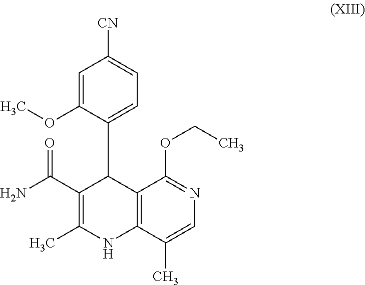
via electrochemical reduction of the compound according to formula (XVII),
wherein in step a) the current density used is decreased or stepwise decreased.

2. The sequential one-pot synthesis according to claim 1, wherein in step a) the current density used in oxidation in step a) is lower than the current density used in step b).

3. The sequential one-pot synthesis according claim 1, wherein
step a) is conducted at temperatures selected from 15 to 150° C., 15 to 120° C., and 20 to 100° C.; and/or
in step a) first the electrochemical oxidation is conducted and then the reaction mixture is heated; and/or
in step a) first the electrochemical oxidation is conducted and then the reaction mixture is heated to temperature selected from 50 to 150° C., 75 to 150° C., to 80 to 150° C., 80 to 140° C. and 90 to 120° C.

4. The sequential one-pot synthesis according to claim 1, wherein in step a) and/or b) a conducting salt is used and the conducting salt is selected from the group of organic ammonia salts, ionic liquids, tetraalkylammonium fluoroborates, quaternary ammonia salts and mixtures thereof; or is selected from the group of tetraethylammonium tetrafluoroborate, tetrabutylammonium hexafluorophosphate, tetrabutylammonium hydrogen sulfate and mixtures thereof.

5. The sequential one-pot synthesis according to claim 1, wherein
in step a) 0.05 to 1 equivalents of conducting salt is used, based on 1 equivalent of the compound according to formula ent-(I); or
in step b) 0.05 to 3 equivalents of conducting salt is used, based on 1 equivalent of the compound according to formula (XIII).

6. The sequential one-pot synthesis according claim 1, wherein in step a) and/or b) the solvent is selected from
aprotic solvents, protic solvents, and mixtures thereof or is selected from polar aprotic solvents; or
polar aprotic solvents selected from dimethylformamide (DMF), dimethyl sulfoxide, acetonitrile, acetone; or
protic solvents selected from methanol, acetic acid, ethanol, formic acid, water and mixtures thereof; or
dimethylformamide (DMF), methanol, acetic acid and mixtures thereof; or
methanol, acetic acid and mixtures thereof; or
methanol; or
acetic acid;
or mixtures of the foregoing.

7. The sequential one-pot synthesis according to claim 1, wherein in step a) an acid is used or wherein the acid used is selected from
organic acids, sulfonic acids, organic polar acids and mixtures thereof,
acetic acid, formic acid and mixtures thereof is used;
acetic acid;
and mixtures thereof.

8. The sequential one-pot synthesis according to claim 1, wherein in step a)
0.1 to 10 equivalents of the acid is used; or
0.1 to 5 equivalents of the acid is used; or
1.5 to 2 equivalents of the acid is used; or
1.7 equivalents of the acid is used,
based on 1 equivalent of the compound according to formula (XIII).

9. The sequential one-pot synthesis according to claim 1, wherein in step a)
the electrochemical oxidation is carried out at a cell voltage from 0.1 to 50 V or is less than 50 V; and/or
the electrochemical oxidation is carried out at a cell voltage from 0.1 to 20 V or is less than 20 V; and/or
the electrochemical oxidation is carried out at current densities selected from 1 to 30000 A/m², 1 to 20000 A/m², 1 to 10000 A/m², and 1 to 500 A/m²; and/or
the electrochemical oxidation is carried out at current densities selected from 1 to 5000 A/m², 1 to 500 A/m², 1 to 100 A/m², 1 to 50 A/m², 5 to 5000 A/m², 5 to 500 A/m², 5 to 100 A/m², 5 to 50 A/m², 10 to 5000 A/m², 10 to 500 A/m², 10 to 100 A/m², and 10 to 50 A/m².

10. The sequential one-pot synthesis according to claim 1, wherein in step b)
the electrochemical reduction is carried out at a cell voltage from 0.1 to 60 V or is less than 60 V; and/or
the electrochemical reduction is carried out at a cell voltage from 0.1 to 30 V or is less than 30 V; and/or
the electrochemical reduction is carried out at current densities selected from 1 to 30000 A/m², 1 to 25000 A/m², 1 to 20000 A/m², 1 to 15000 A/m², 1 to 10000 A/m² and 1 to 5000 A/m²; and/or
the electrochemical reduction is carried out at current densities selected from 1 to 10000 A/m², 1 to 1000 A/m², 1 to 500 A/m², 5 to 10000 A/m², 50 to 1000 A/m², 200 to 500 A/m², 10 to 10000 A/m², 10 to 1000 A/m², 10 to 500 A/m², 100 to 500 A/m², 200 to 400 A/m², 200 A/m², 250 A/m², 300 A/m², 350 A/m² and 400 A/m²; and/or
the electrochemical reduction is carried out at a temperature from 1 to 100° C., 1 to 50° C., 10 to 35° C., 15 to 30° C., or at ambient temperature; and/or
the electrochemical reduction is carried out at a temperature from 1 to 50° C., 10 to 35° C., 15 to 30° C., or at ambient temperature.

11. The sequential one-pot synthesis according claim 1, wherein in step a) and/or b) a pressure of approximately 0.5 bar to 10 bar is used, wherein optionally the pressure used in step a) and/or b) is decreased, increased, constant or varying.

12. The sequential one-pot synthesis according to claim 1, wherein in step a) and/or b)
the flow rate decreased, increased, constant or varying; and/or
the flow rate is 0.01 mL/min to 10000 L/min; and/or
a pressure of 0.1 to 10 bar is used.

13. A process for synthesizing (4S)-4-(4-cyano-2-methoxyphenyl)-5-ethoxy-2,8-dimethyl-1,4-dihydro-1,6-naphthyridine-3-carboxamide of formula (I) 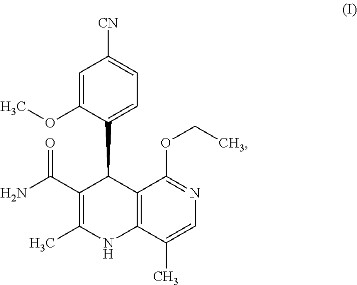
the synthesis comprising the steps of:
Step (1) a sequential one-pot synthesis for synthesizing 4-(4-cyano-2-methoxy-phenyl)-5-ethoxy-2,8-dimethyl-1,4-dihydro-1,6-naphthyridine-3-carboxamide according to formula (XIII) 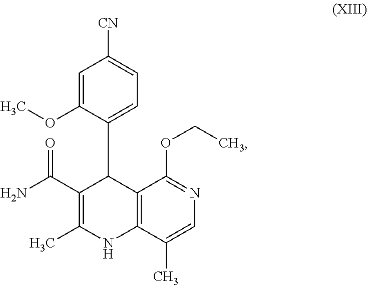
the synthesis comprising steps a) and b):
step a) synthesizing 4-(4-cyano-2-methoxy-phenyl)-5-ethoxy-2,8-dimethyl-1,6-naphthyridine-3-carboxamide according to formula (XVII) 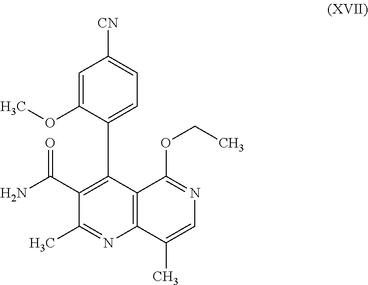
via electrochemical oxidation of (4R)-4-(4-cyano-2-methoxy-phenyl)-5-ethoxy-2,8-dimethyl-1,4-dihydro-1,6-naphthyridine-3-carboxamide according to formula ent-(I) 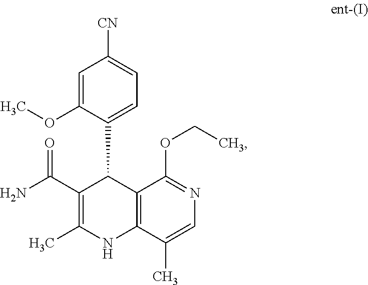
step b) synthesizing 4-(4-cyano-2-methoxy-phenyl)-5-ethoxy-2,8-dimethyl-1,4-dihydro-1,6-naphthyridine-3-carboxamide according to formula (XIII) 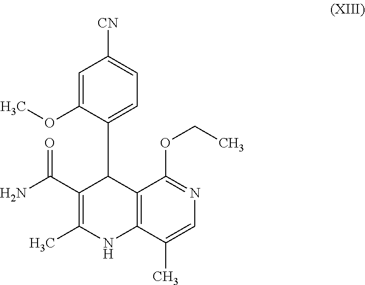
via electrochemical reduction of the compound according to formula (XVII)
and
Step (2) isolating the compound according to formula (I) from the compound according to formula (XIII),
wherein optionally step (1) is characterized by claim 1.

14. The process according to claim 13, wherein (4S)-4-(4-cyano-2-methoxyphenyl)-5-ethoxy-2,8-dimethyl-1,4-dihydro-1,6-naphthyridine-3-carboxamide of formula (I) is obtained in enantiomeric excess e.e. % of >99%.

15. The process according to claim 13, wherein
(4S)-4-(4-cyano-2-methoxyphenyl)-5-ethoxy-2,8-dimethyl-1,4-dihydro-1,6-naphthyridine-3-carboxamide of formula (I) is obtained in enantiomeric excess e.e. % of <1.5%; and/or
(4S)-4-(4-cyano-2-methoxyphenyl)-5-ethoxy-2,8-dimethyl-1,4-dihydro-1,6-naphthyridine-3-carboxamide of formula (I) in step (1) and/or (2) is obtained in amount of 0.01 to 100 mm/mL, based on the total volume of the reaction mixture.
